(19) United States
(12) Patent Application Publication
(10) Pub. No.: US 2009/0325971 A1
(43) Pub. Date: Dec. 31, 2009

(54) Pentacyclic Alkaloid Compounds and Methods of Use Thereof

(75) Inventors: Samuel J. Danishefsky, Englewood, NJ (US); Collin Chan, New York, NY (US)

Correspondence Address: WilmerHale/Columbia University, 399 PARK AVENUE, NEW YORK, NY, 10022, US

(73) Assignee: The Trustees Of Columbia University In The City Of New York, New york, NY (US)

(21) Appl. No.: 11/885,287

(22) PCT Filed: Feb. 28, 2006
(86) PCT No.: PCT/US06/07177

§ 371 Date: Aug. 10, 2009

Related U.S. Application Data
(60) Provisional application No. 60/657,933, filed on Mar. 2, 2005.
(60) Provisional application No. 60/690,693, filed on Jun. 15, 2005.

(51) Int. Cl. A61K 31/4985; C07D 471/14; A61P 35/00

(52) U.S. Cl. 514/250; 544/342

(57) ABSTRACT

The present invention relates to Pentacyclic Alkaloid Compounds, compositions comprising an effective amount of a Pentacyclic Alkaloid Compound and methods for treating or preventing cancer, a bacterial infection, a fungal infection, or a yeast infection, comprising administering to a subject in need thereof an effective amount of a Pentacyclic Alkaloid Compound. The present invention also relates to compounds and methods that are useful for making Cribrostatin IV.

This application claims the benefit of U.S. Provisional Application No. 60/657,933, filed Mar. 2, 2005, and of U.S. Provisional Application No. 60/690,693, filed Jun. 15, 2005, the disclosure of each of which is incorporated by reference herein in its entirety.

An invention disclosed herein was made with U.S. Government support from the National Institutes of Health (Grant No. HL25848). Accordingly, the U.S. Government may have certain rights in the invention.

### 1. FIELD OF THE INVENTION

The present invention relates to Pentacyclic Alkaloid Compounds, compositions comprising an effective amount of a Pentacyclic Alkaloid Compound and methods for treating or preventing a bacterial infection, a fungal infection, a yeast infection, or cancer, comprising administering to a subject in need thereof an effective amount of a Pentacyclic Alkaloid Compound. The present invention also relates to compounds and methods that are useful for making Cribrostatin IV.

### 2. BACKGROUND OF THE INVENTION

Optically active organic molecules have important utility, especially as pharmaceutically active compounds and as intermediates for the synthesis of complex organic molecules. As such, the development of synthetic methodologies that allow organic chemists to make optically active compounds in a planned manner are of great importance.

The ability to carry out chemical reactions with a high degree of stereoselective control allows a synthetic chemist access to optically active compounds of increasing complexity.

The cribrostatins are a family of pentacyclic alkaloid compounds that inhibit the growth of cancer cells. Due to their biological activity and complex molecular structure, the cribrostatins have attracted the interest of synthetic chemists, medicinal chemists and biologists.

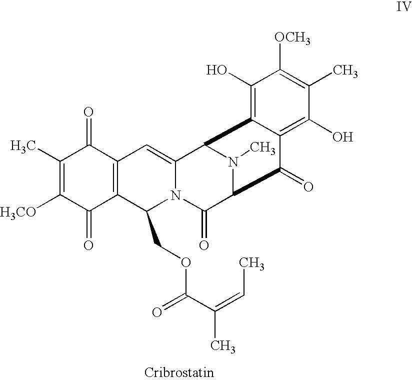

Cribrostatin IV is a potent cytotoxic agent that has been isolated from a blue marine sponge, Cribrochalina, in reef passages off the Republic of Maldives.

Although methods have been reported for the total synthesis of various polycyclic alkaloids, there remains a need in the art for synthetic methods that provide access to Cribrostatin IV. There also remains a need for anticancer agents with improved efficacy and safety profiles.

### 3. SUMMARY OF THE INVENTION

In one aspect the invention provides a method for making a compound having the formula:

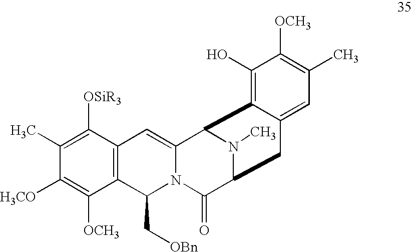

the method comprising allowing a compound of formula 34:

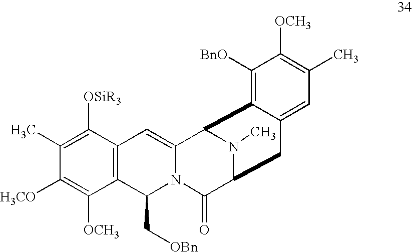

wherein each R is independently —C₁-C₁₂ alkyl or phenyl,
to react with H₂ in the presence of Pd/C under conditions that are sufficient to make the compound of formula 35.

In another aspect the invention provides a method for making a compound having the formula:

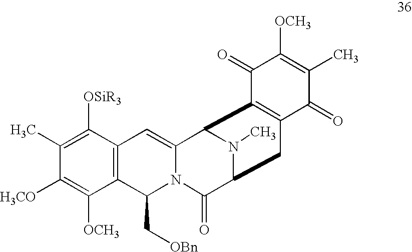

the method comprising allowing a compound of formula 35:

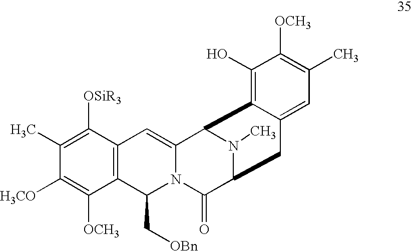

wherein each R is independently —C₁-C₁₂ alkyl or phenyl,
to react with (a) (KSO₃)₂NO, (b) DDQ, or (c) O₂ in the presence of a metal, under conditions that are sufficient to make the compound of formula 36.

In another aspect the invention provides a method for making a compound having the formula:

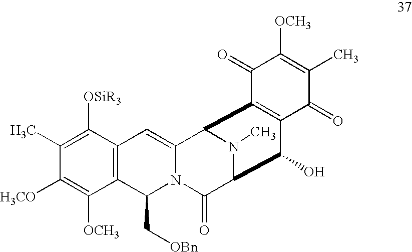

the method comprising allowing a compound of formula 36:

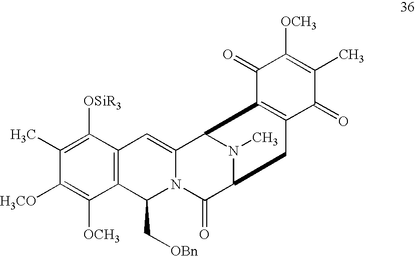

wherein each R is independently —C₁-C₁₂ alkyl or phenyl,
to react with SeO₂ under conditions that are sufficient to make the compound of formula 37.

In still another aspect the invention provides a method for making a compound having the formula:

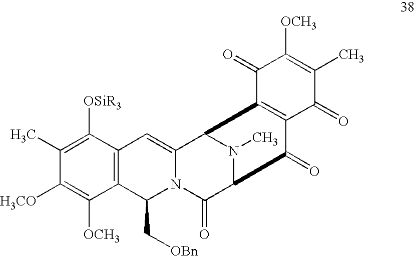

the method comprising allowing a compound of formula 37:

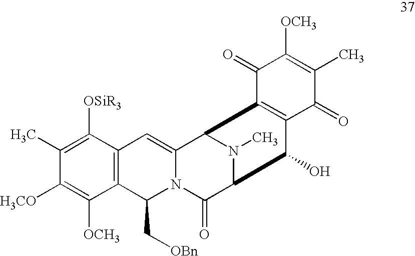

wherein each R is independently —C₁-C₁₂ alkyl or phenyl,
to react with an oxidizing agent under conditions that are sufficient to make the compound of formula 38.

In yet another aspect the invention provides a method for making a compound having the formula:

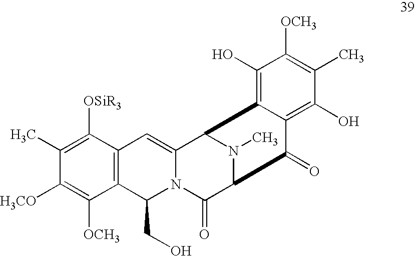

the method comprising allowing a compound of formula 38:

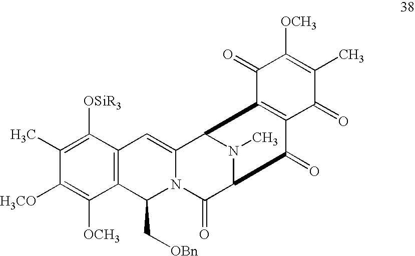

wherein each R is independently —C₁-C₁₂ alkyl or phenyl,
to react with H₂ in the presence of Pd/C under conditions that are sufficient to make the compound of formula 39.

In a further aspect the invention provides a method for making a compound having the formula:

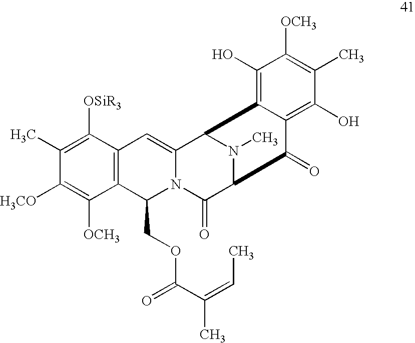

the method comprising allowing a compound of formula 39:

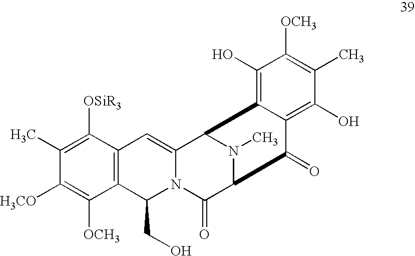

wherein each R is independently —C₁-C₁₂ alkyl or phenyl,
to react with a compound of formula 40:

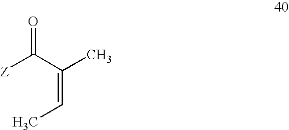

wherein Z is —Cl, —Br, —OH, —C(O)(C₁-C₁₂ alkyl), —C(O)-phenyl, or

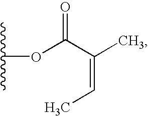

wherein phenyl is unsubstituted or substituted with up to 3 substituents independently selected from -halo, —C₁-C₁₂ alkyl, —O—(C₁-C₁₂ alkyl), —CN, —CF₃, or —NO₂,
under conditions that are sufficient to make the compound of formula 41.

In a further aspect the invention provides a method for making a compound having the formula:

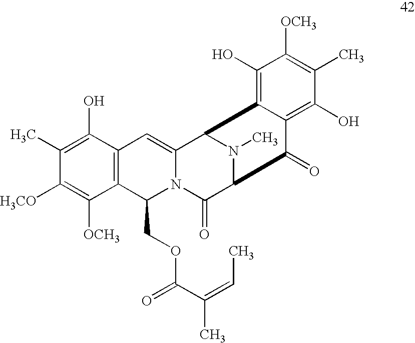

the method comprising allowing a compound of formula 41:

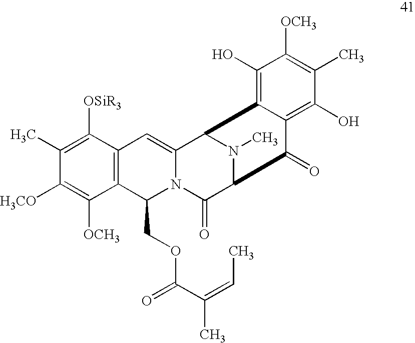

wherein each R is independently —C₁-C₁₂ alkyl or phenyl,
to react with a Bronsted acid or a fluoride salt under conditions that are sufficient to make Compound 42.

In another aspect the invention provides a method for making a compound having the formula:

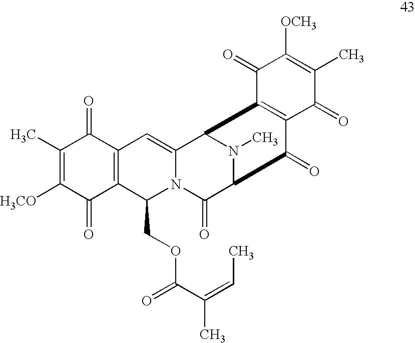

the method comprising allowing Compound 42:

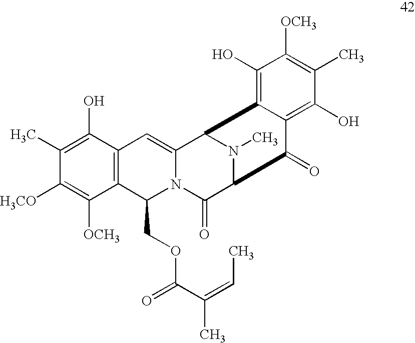

to react with (a) silver oxide, (b) phenyliodonium(III) diacetate, (c) ceric(IV) ammonium nitrate, (d) (KSO₃)₂NO, (e) PhSeCl, (f) MnO₂, (g) phenyliodine (III) bis(trifluoroacetate), or (h) O₂ in the presence of a metal, under conditions that are sufficient to make Compound 43.

In still another aspect the invention provides a method for making a compound having the formula:

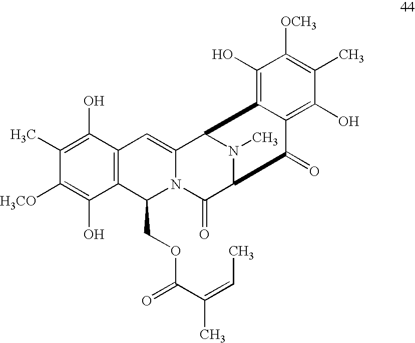

the method comprising allowing Compound 43:

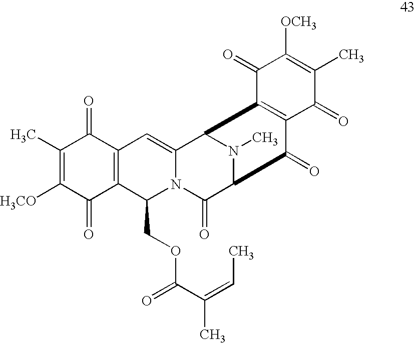

to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44.

In yet another aspect the invention provides a method for making Cribrostatin IV,

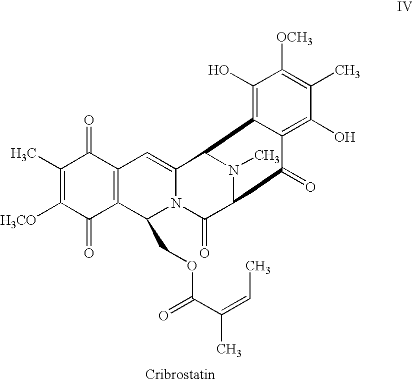

the method comprising allowing Compound 44:

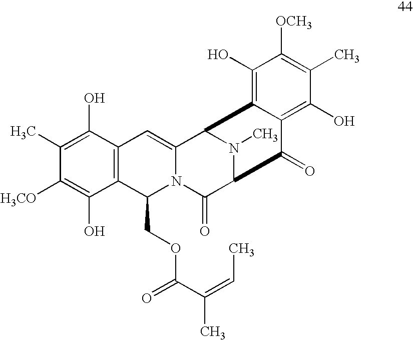

to react with O₂ under conditions that are sufficient to make Cribrostatin IV.

A compound of formula 34-39 or 41-44 is useful for making Cribrostatin IV.

In another aspect the invention provides a method for making a compound having the formula:

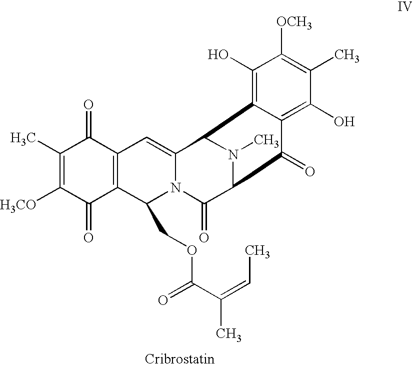

the method comprising the steps:
(i) allowing a compound of formula 34:

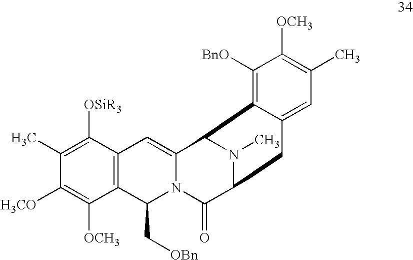

wherein each R is independently —C₁-C₁₂ alkyl or phenyl,
to react with H₂ in the presence of Pd/C under conditions that are sufficient to make a compound of formula 35:

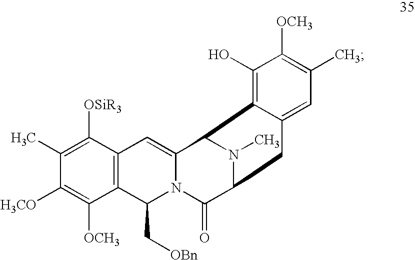

(ii) allowing a compound of formula 35 to react with (a) (KSO₃)₂NO, (b) DDQ, or (c) O₂ in the presence of a metal, under conditions that are sufficient to make a compound of formula 36:

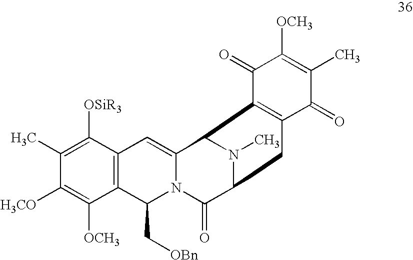

wherein each R is independently —C₁-C₁₂ alkyl or phenyl,
(iii) allowing a compound of formula 36 to react with SeO₂ under conditions that are sufficient to make a compound of formula 37:

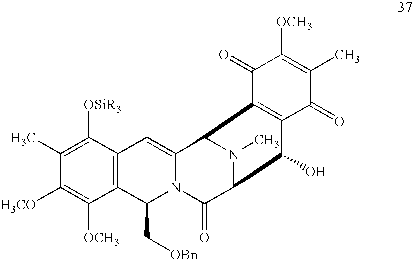

wherein each R is independently —C₁-C₁₂ alkyl or phenyl,
(iv) allowing a compound of formula 37 to react with an oxidizing agent under conditions that are sufficient to make a compound of formula 38:

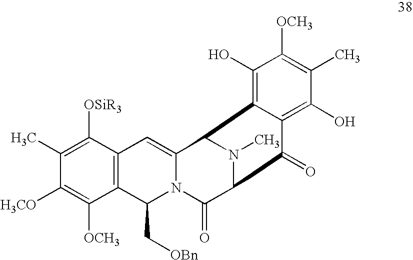

wherein each R is independently —C₁-C₁₂ alkyl or phenyl,
(v) allowing a compound of formula 38 to react with H₂ in the presence of Pd/C under conditions that are sufficient to make a compound of formula 39:

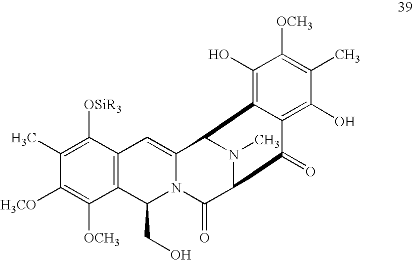

wherein each R is independently —C₁-C₁₂ alkyl or phenyl,
(vi) allowing a compound of formula 39 to react with a compound of formula 40:

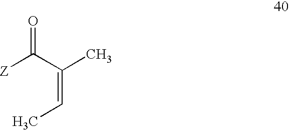

wherein Z is —Cl, —Br, —OH, —C(O)(C₁-C₁₂ alkyl), —C(O)-phenyl, or

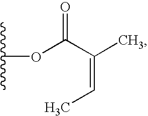

wherein phenyl is unsubstituted or substituted with up to 3 substituents independently selected from -halo, —C₁-C₁₂ alkyl, —O—(C₁-C₁₂ alkyl), —CN, —CF₃, or —NO₂, under conditions that are sufficient to make a compound of formula 41:

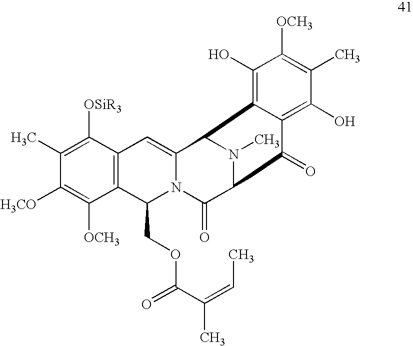

wherein each R is independently —C₁-C₁₂ alkyl or phenyl,
(vii) allowing a compound of formula 41 to react with a Bronsted acid or a fluoride salt under conditions that are sufficient to make Compound 42:

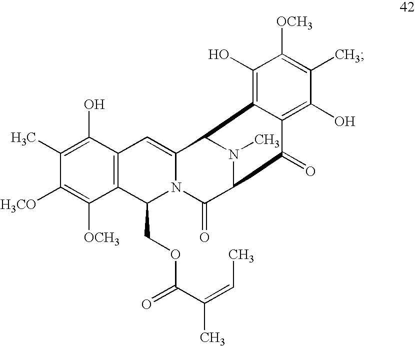

(viii) allowing Compound 42 to react with (a) silver oxide, (b) phenyliodonium(III) diacetate, (c) ceric(IV)ammonium nitrate, (d) (KSO₃)₂NO, (e) PhSeCl, (D) MnO₂, (g) phenyliodine (III) bis(trifluoroacetate), or (h) O₂ in the presence of a metal, under conditions that are sufficient to make Compound 43:

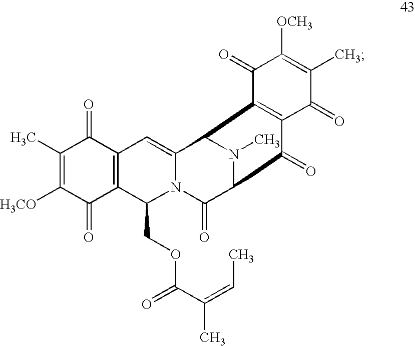

(ix) allowing Compound 43 to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44:

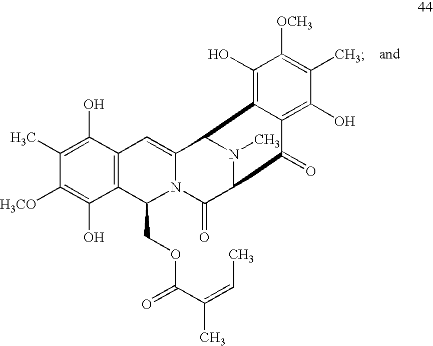

(x) allowing Compound 44 to react with O₂ under conditions that are sufficient to make Cribrostatin IV.

In one aspect the invention provides a compound having the formula:

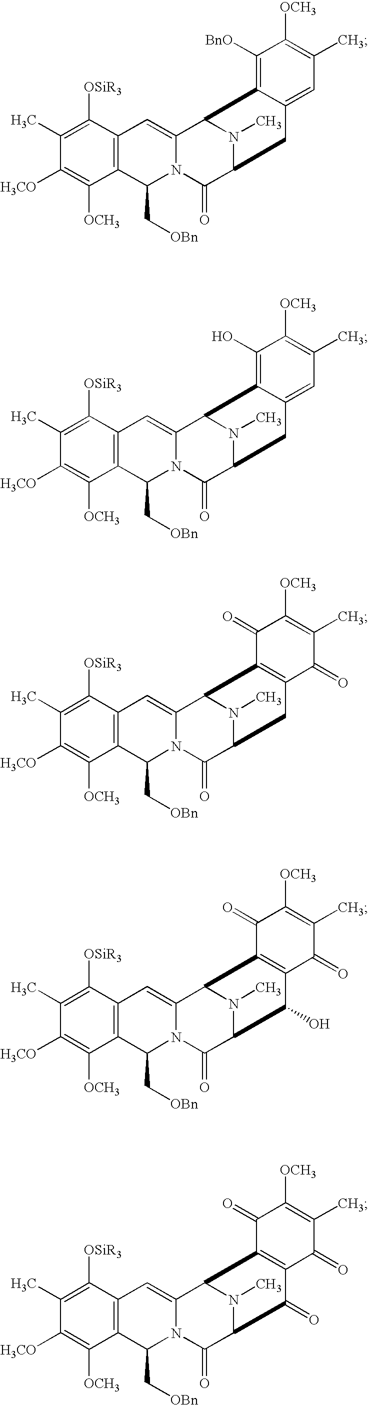

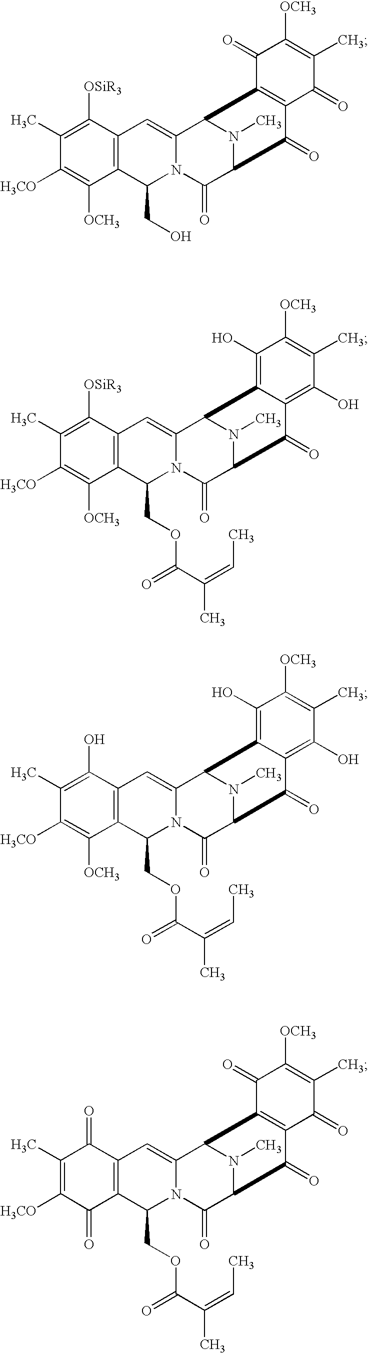

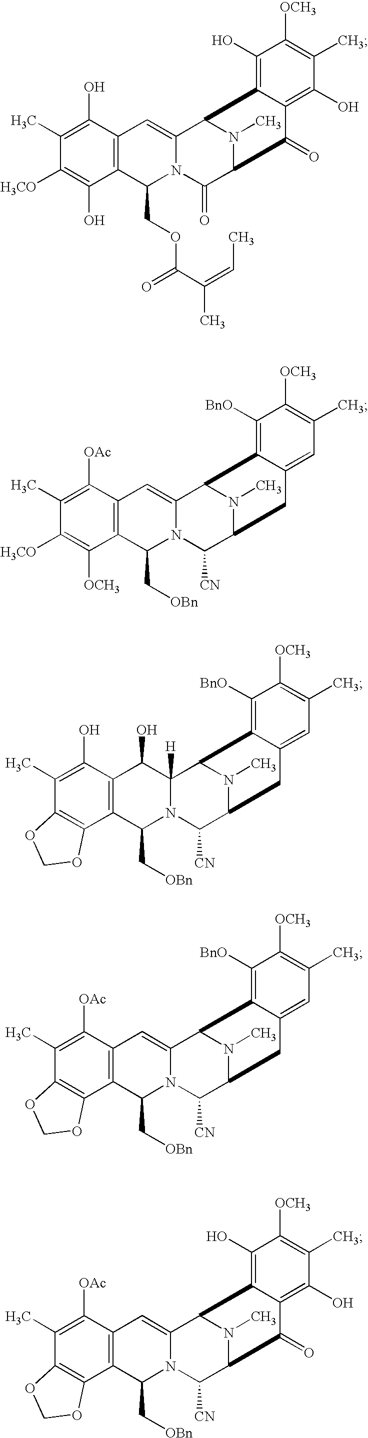

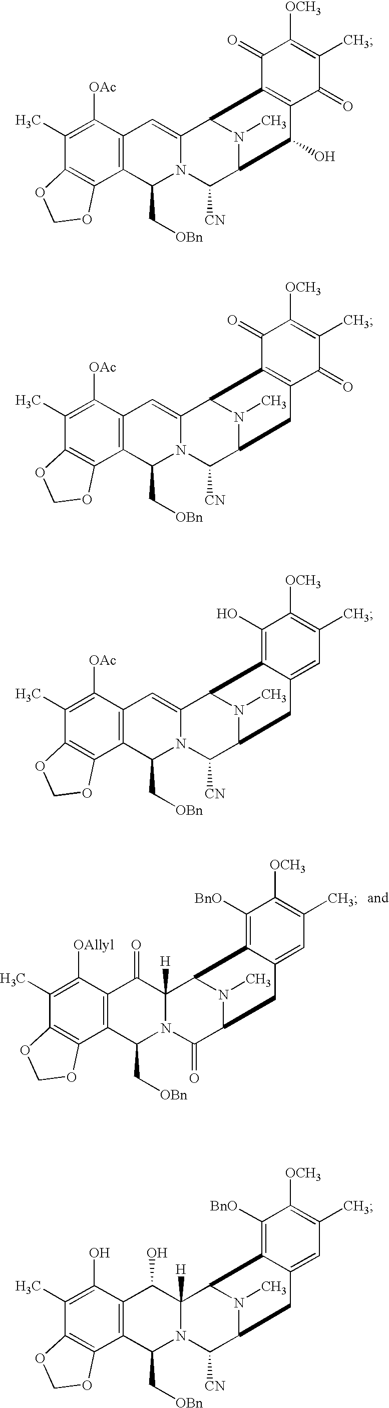

wherein each occurrence of R is independently —C₁-C₁₂ alkyl or -phenyl.

In one aspect, the present invention provides compounds having the Formula (I):

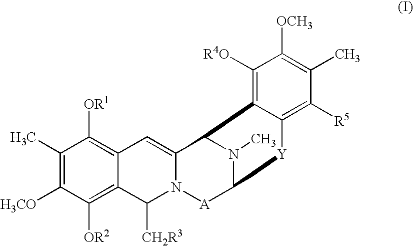

and pharmaceutically acceptable salts thereof,
wherein:

R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R² is —H or —C₁-C₁₂ alkyl;

R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl, or

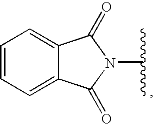

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R⁴ is —H, —C₁-C₁₂ alkyl or -benzyl;

R⁵ is —H, —OH or —O—C₁-C₁₂ alkyl;

A is —CH₂—, —CH(α-OH)—, —CH(α-CN)— or —C(O)—; and

Y is —CH₂—, —CH(α-OH)— or —C(O)—.

In another aspect, the present invention provides compounds having the Formula (II):

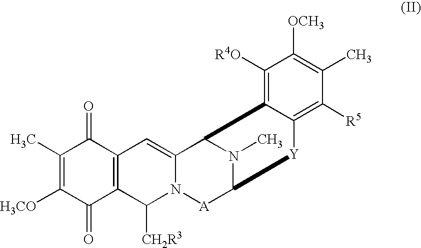

and pharmaceutically acceptable salts thereof,
wherein:

R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl, —O-benzyl or

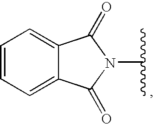

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R⁴ is —H, —C₁-C₁₂ alkyl or -benzyl;

R⁵ is —H, —OH or —O—C₁-C₁₂ alkyl;

A is —CH₂—, —CH(α-OH)—, —CH(α-CN)— or —C(O)—; and

Y is —CH₂—, —CH(α-OH)— or —C(O)—.

In still another aspect, the present invention provides compounds having the Formula (III):

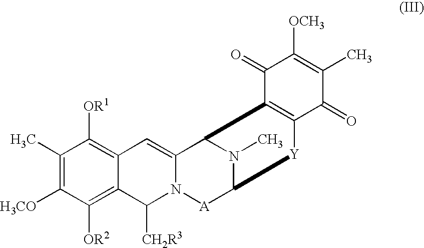

and pharmaceutically acceptable salts thereof,
wherein:

R¹ is —H, —C₁-C₁₂ alkyl, -allyl, —C(O)—(C₁-C₁₂ alkyl), —C(O)-aryl, or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R² is —H or —C₁-C₁₂ alkyl;

R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or

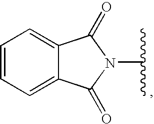

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

A is —CH₂—, —CH(α-OH)—, —CH(α-CN)— or —C(O)—; and

Y is —CH₂—, —CH(α-OH)— or —C(O)—.

In yet another aspect, the present invention provides compounds having the Formula (IV):

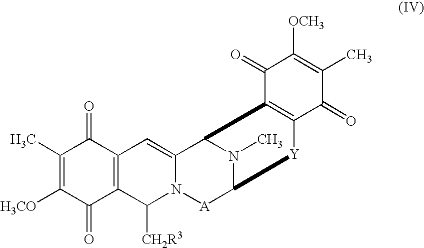

and pharmaceutically acceptable salts thereof,
wherein:

R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl, —O-benzyl or

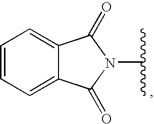

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

A is —CH₂—, —CH(α-OH)—, —CH(α-CN)— or —C(O)—; and

Y is —CH₂—, —CH(α-OH)— or —C(O)—.

In a further aspect, the present invention provides compounds having the Formula (V):

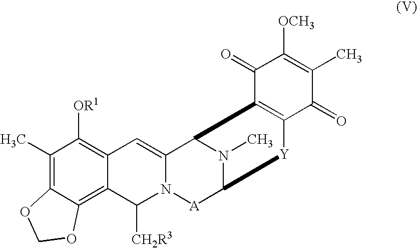

and pharmaceutically acceptable salts thereof,
wherein:

R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or

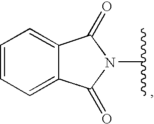

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

A is —CH₂—, —CH(α-OH)—, —CH(α-CN)— or —C(O)—; and

Y is —CH₂—, —CH(α-OH)— or —C(O)—.

In another aspect, the present invention provides compounds having the Formula (VI):

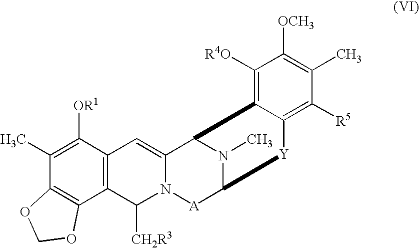

and pharmaceutically acceptable salts thereof,
wherein:

R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or

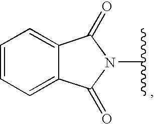

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R⁴ is —H or —C₁-C₁₂ alkyl or -benzyl;

R¹ is —H, —OH or —O—C₁-C₁₂ alkyl;

A is —CH₂—, —CH(α-OH)—, —CH(α-CN)— or —C(O)—; and

Y is —CH₂—, —CH(α-OH)— or —C(O)—.

In still another aspect, the present invention provides compounds having the Formula (VII):

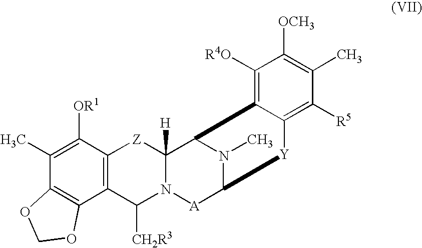

and pharmaceutically acceptable salts thereof,
wherein:

R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or

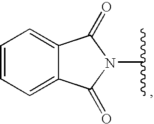

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;

R⁴ is —H or —C₁-C₁₂ alkyl or -benzyl;

R⁵ is —H, —OH or —O—C₁-C₁₂ alkyl;

A is —CH₂—, —CH(α-OH)—, —CH(α-CN)— or —C(O)—;

Y is —CH₂—, —CH(α-OH)— or —C(O)—; and

Z is —C(O)— or —CH(OH)—.

A compound of Formula (I), (II), (III), (IV), (V), (VI) or (VII) or a pharmaceutically acceptable salt thereof (a “Pentacyclic Alkaloid Compound”) is useful for treating or preventing a bacterial infection, a fungal infection, a yeast infection or cancer (a “Condition”) in a subject.

The invention also relates to compositions comprising an amount of a Pentacyclic Alkaloid Compound that is effective to treat or prevent a Condition, and a physiologically acceptable carrier or vehicle. The compositions are useful for treating or preventing a Condition in a subject.

The invention further relates to methods for treating or preventing a Condition, comprising administering to a subject in need thereof an amount of a Pentacyclic Alkaloid Compound that is effective to treat or prevent a Condition.

### 4. DETAILED DESCRIPTION OF THE INVENTION

### 4.1 Definitions and Abbreviations

The terms used herein have the following meaning:

The term “—C₁-C₆ alkyl” as used herein, refers to a straight chain or branched non-cyclic saturated hydrocarbon having from 1 to 6 carbon atoms, wherein one of the hydrocarbon's hydrogen atoms has been replaced by a single bond. Representative straight chain —C₁-C₆ alkyls include -methyl, -ethyl, -n-propyl, -n-butyl, -n-pentyl and -n-hexyl. Representative branched —C₁-C₆ alkyls include -isopropyl, -sec-butyl, -isobutyl, -tert-butyl, -isopentyl, -neopentyl, -1-methylbutyl, -isohexyl, -neohexyl, -2-methylbutyl, -3-methylbutyl, -1,1-dimethylpropyl and -1,2-dimethylpropyl.

The term “—C₁-C₁₂ alkyl” as used herein, refers to a straight chain or branched non-cyclic saturated hydrocarbon having from 1 to 12 carbon atoms, wherein one of the hydrocarbon's hydrogen atoms has been replaced by a single bond. Representative —C₁-C₁₂ alkyls include methyl, ethyl, propyl, butyl, pentyl, hexyl, heptyl, octyl, nonyl, decyl, undecyl, dodecyl, isopropyl, isobutyl, sec-butyl and tert-butyl, isopentyl, neopentyl, isohexyl, isoheptyl, isooctyl, isononyl, isodecyl, isoundecyl and isododecyl.

The term “allyl” as used herein, refers to the group having the formula: —CH₂—CH═CH₂.

The term “aryl” as used herein, refers to a phenyl or naphthyl group. In one embodiment, an aryl group is substituted with one or more of: -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

The term “benzyl” as used herein refers to the group having the formula: —CH₂-phenyl.

The term “halo” as used herein, refers to —F, —Cl, —Br, or —I.

A “subject” is a mammal, e.g., a human, mouse, rat, guinea pig, dog, cat, horse, cow, pig, or non-human primate, such as a monkey, chimpanzee, baboon or rhesus. In one embodiment, the subject is a human.

Representative “pharmaceutically acceptable salts” include, e.g., acetate, amsonate (4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fumarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A hydrate is another example of a pharmaceutically acceptable salt.

An “effective amount” when used in connection with a Pentacyclic Alkaloid Compound is an amount that is effective for treating or preventing a Condition.

An “effective amount” when used in connection with another anticancer agent is an amount that is effective for treating or preventing cancer alone or in combination with a Pentacyclic Alkaloid Compound. “In combination with” includes administration within the same composition and within different compositions. In the latter instance, the anticancer agent is administered during a time when the Pentacyclic Alkaloid Compound exerts its prophylactic or therapeutic effect, or vice versa.

An “effective amount” when used in connection with an antiemetic agent is an amount that is effective for preventing or lessening the incidence of emesis.

The term “—CH(α-OH)-” means that the —OH group is below the plane of the Pentacyclic Alkaloid Compound as depicted and has the structure:

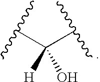

The term “—CH(β-OH)-” means that the —OH group is above the plane of the Pentacyclic Alkaloid Compound as depicted and has the structure:

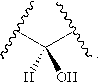

The term “—CH(α-CN)-” means that the —CN group is below the plane of the Pentacyclic Alkaloid Compound as depicted and has the structure:

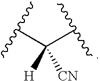

Some chemical structures herein are depicted using bold and dashed lines to represent chemical bonds. These bold and dashed lines depict absolute stereochemistry.

The following abbreviations are used herein and have the indicated definitions: AcOH is acetic acid, Bn is benzyl, BnBr is benzyl bromide, Boc is tert-butoxy carbonyl, BOP is benzotriazole-1-yl-oxy-tris(dimethylamino)phosphonium hexafluorophosphate, BOPCl is benzotriazole-1-yl-oxy-tris(dimethylamino)phosphonium hexafluorophosphonyl chloride, Bu₃SnH is tributyltinhydride, Bu₄NBr is tetrabutylammonium bromide, n-BuLi is n-butyllithium, CSA is camphorsulfonic acid, DBU is 1,8-diazabicyclo[5.4.0]undecane, DDQ is 2,3-dichloro-5,6-dicyano-1,4-benzoquinone, Dess-Martin periodinane is 1,1,1-Triacetoxy-1,1-dihydro-1,2-benziodoxol-3(1H)-one, DMAP is 4-dimethylaminopyridine, DMF is N,N-dimethylformamide, DPPA is diphenyl phosphoryl azide, Et₂AlCl is diethyl aluminum chloride, EtOAc is ethyl acetate, EtOH is ethanol, KCN is potassium cyanide, LiAlH₄ is lithium aluminumhydride, mCPBA is m-chloroperoxybenzoic acid, 2,6-lutidine is 2,6-dimethylpyridine, Me is methyl, MeI is methyl iodide, MeOH is methanol, MS is mass spectrometry, NMR is nuclear magnetic resonance, NaBH₄ is sodium borohydride, NaCl is sodium chloride, NaHCO₃ is sodium bicarbonate, NH₄Cl is ammonium chloride, PCC is pyridinium chlorochromate, Pd/C is palladium on carbon, Ph is phenyl, PMB is p-methoxybenzyl, PMBC1 is p-methoxybenzyl chloride, PIFA is phenyliodine (III) bis(trifluoroacetate), PTLC is preparative thin-layer chromatography, t-Bu is tert-butyl, TBAB is tetrabutylammonium borane, TBAF is tetra-n-butylammonium fluoride, TBDPSC1 is tert-butyl diphenylsilyl chloride, TBDPS is tert-butyl diphenylsilyl, TBS is tert-butyldimethylsilyl, TBSOTf is tert-butyldimethylsilyl trifluoromethane sulfonate, TEA is triethylamine, THF is tetrahydrofuran, Ts is p-toluenesulfonyl and TsCl is p-toluenesulfonyl chloride.

### 4.2 Methods For Making a Compound of Formula 35

In one embodiment, the invention provides a method for making a compound of formula 35:

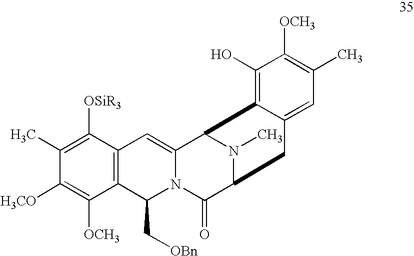

the method comprising allowing a compound of formula 34:

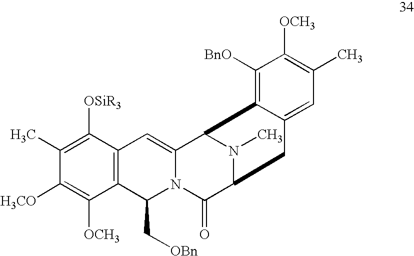

wherein each R is independently —C₁-C₁₂ alkyl or phenyl,

to react with H₂ in the presence of Pd/C under conditions that are sufficient to make the compound of formula 35.

In one embodiment the Pd/C has from about 1% palladium to about 20% palladium by weight of the Pd/C.

In another embodiment the Pd/C has from about 5% palladium to about 10% palladium by weight of the Pd/C.

In a specific embodiment, the Pd/C has about 5% palladium by weight of the Pd/C.

In one embodiment, the weight-to-weight ratio (w/w) of Pd/C to a compound of Formula 34 is from about 1% to about 20%.

In one embodiment, the weight-to-weight ratio (w/w) of Pd/C to a compound of Formula 34 is from about 5% to about 15%.

In one embodiment, the weight-to-weight ratio (w/w) of Pd/C to a compound of Formula 34 is about 10%.

The method can be carried out in the presence of a reaction solvent, such as ethyl acetate, methanol, ethanol, isopropanol, tert-butanol, benzene, toluene, THF or mixtures thereof.

In one embodiment, the solvent is ethyl acetate.

In still another embodiment, the solvent is substantially anhydrous, i.e., comprises less than about 1% water.

In one embodiment, the method is carried out for a time of about 1 hour to about 72 hours.

In another embodiment, the method is carried out for a time of about 12 hours to about 48 hours.

In yet another embodiment, the method is carried out for a time of about 18 hours to about 36 hours.

In yet another embodiment, the method is carried out for a time of about 24 hours.

In one embodiment, the method is carried out at a temperature of about 0° C. to about 60° C.

In another embodiment, the method is carried out at a temperature of about 10° C. to about 40° C.

In still another embodiment, the method is carried out at a temperature of about 25° C.

Typically H₂ gas is bubbled through a composition comprising a compound of formula 34, Pd/C and a reaction solvent. Alternatively, an atmosphere of H₂ is maintained over the composition.

In one embodiment an atmosphere of H₂ is maintained over the composition via a balloon containing H₂.

In one embodiment, a mixture of a compound of formula 34, Pd/C, and a reaction solvent can be filtered to remove Pd/C, then concentrated in vacuo.

### 4.3 Methods For Making a Compound of Formula 36

In one embodiment, the invention provides a method for making a compound of formula 36:

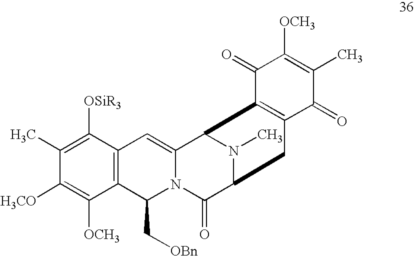

the method comprising allowing a compound of formula 35:

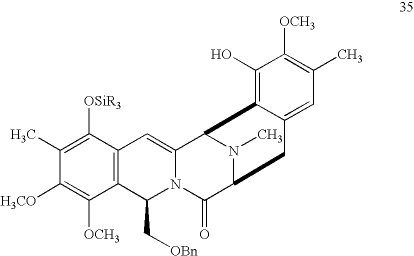

wherein each R is independently C₁-C₁₂ alkyl or phenyl,
to react with (a) (KSO₃)₂NO, (b) DDQ, or (c) O₂ in the presence of a metal, under conditions that are sufficient to make the compound of formula 36.

In one embodiment, the compound of formula 35 is allowed to react with DDQ.

In another embodiment, the compound of formula 35 is allowed to react with (KSO₃)₂NO.

In another embodiment, the compound of formula 35 is allowed to react with O₂ in the presence of a metal.

Metals useful in the present methods include, but are not limited to copper(I), copper (II), iron (II), manganese (II), manganese (VII), and cobalt (III).

In another embodiment, when the reagent is (KSO₃)₂NO, the method can further comprise allowing a compound of formula 35 to react in the presence of KH₂PO₄.

In one embodiment, about 1 to about 10 equivalents of a reagent are used relative to about 1 equivalent of a compound of Formula 35.

In another embodiment, about 2 to about 6 equivalents of a reagent are used relative to about 1 equivalent of a compound of Formula 35.

In another embodiment, about 3 equivalents of a reagent are used relative to about 1 equivalent of a compound of Formula 35.

In one embodiment, about 1 to about 5 equivalents of KH₂PO₄ are used relative to about 1 equivalent of (KSO₃)₂NO.

In another embodiment, about 2 to about 3 equivalents of KH₂PO₄ are used relative to about 1 equivalent of (KSO₃)₂NO.

The method can be carried out in the presence of a reaction solvent, such as acetonitrile, ethyl acetate, THF, benzene, toluene, dioxane, methylene chloride, an organic alcohol, acetone, water or mixtures thereof. In one embodiment, the solvent is a mixture of acetonitrile and water.

In another embodiment, when the oxidizing agent is (KSO₃)₂NO, the solvent is a 3:2 mixture of acetonitrile:water.

In still another embodiment, the solvent is substantially anhydrous, i.e., comprises less than about 1% water.

In one embodiment, the method is carried out for a time of about 1 hour to about 96 hours.

In another embodiment, the method is carried out for a time of about 18 hours to about 72 hours.

In yet another embodiment, the method is carried out for a time of about 24 hours to about 48 hours.

In another embodiment, the method is carried out for a time of about 36 hours.

In one embodiment, the method is carried out at a temperature of about 0° C. to about 60° C.

In another embodiment, the method is carried out at a temperature of about 10° C. to about 40° C.

In still another embodiment, the method is carried out at a temperature of about 25° C.

Typically when (KSO₃)₂NO is the oxidizing agent, the method is performed at room temperature in the presence of KH₂PO₄ in the presence of a reaction solvent comprising acetonitrile and water.

In one embodiment, a mixture of compound of formula 36 and a reaction solvent can be mixed with brine and extracted with ethyl acetate, The ethyl acetate solution can be dried over a drying agent, filtered, and concentrated in vacuo to provide a crude product. The crude product can be purified using a variety of chromatographic techniques well known to one skilled in the art of organic synthesis.

### 4.4 Methods For Making a Compound of Formula 37

In one embodiment, the invention provides a method for making a compound of formula 37:

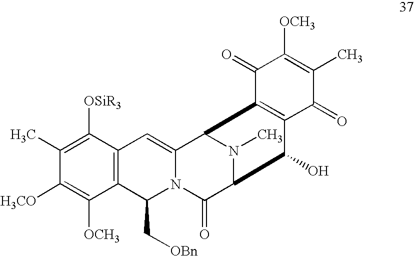

the method comprising allowing a compound of formula 36:

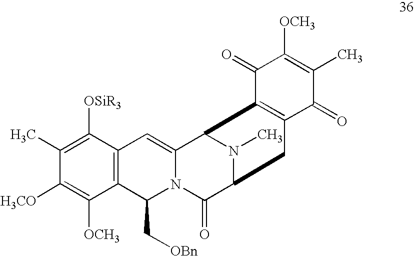

wherein each R is independently —C₁-C₁₂ alkyl or phenyl,
to react with SeO₂ under conditions that are sufficient to make the compound of formula 37.

In one embodiment, about 1 to about 10 equivalents of SeO₂ are used relative to about 1 equivalent of a compound of Formula 36.

In another embodiment, about 2 to about 5 equivalents of SeO₂ are used relative to about 1 equivalent of a compound of Formula 36.

In another embodiment, about 3.5 equivalents of SeO₂ are used relative to about 1 equivalent of a compound of Formula 36.

The method can be carried out in the presence of a reaction solvent, such as dioxane, THF, chloroform, benzene, methylene chloride, or mixtures thereof.

In one embodiment, the solvent is dioxane.

In still another embodiment, the solvent is substantially anhydrous, i.e., comprises less than about 1% water.

In one embodiment, the method is carried out for a time of about 0.5 hours to about 18 hours.

In another embodiment, the method is carried out for a time of about 1 hours to about 12 hours.

In yet another embodiment, the method is carried out for a time of about 2 hours to about 8 hours.

In another embodiment, the method is carried out for a time of about 5 hours.

In one embodiment, the method is carried out at a temperature of about 25° C. to about 100° C.

In another embodiment, the method is carried out at a temperature of about 50° C. to about 75° C.

In still another embodiment, the method is carried out at a temperature of about

Typically the method is performed at a temperature of about 100° C. in the presence of dioxane.

In one embodiment, a mixture of a compound of formula 37 and a reaction solvent can be concentrated in vacuo to provide a crude product that can be purified using a variety of chromatographic techniques which are well-known to one skilled in the art of organic synthesis.

### 4.5 Methods For Making a Compound of Formula 38

In one embodiment, the invention provides a method for making a compound of formula 38:

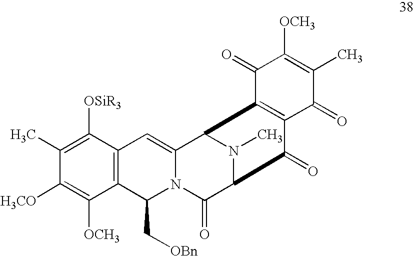

the method comprising allowing a compound of formula 37:

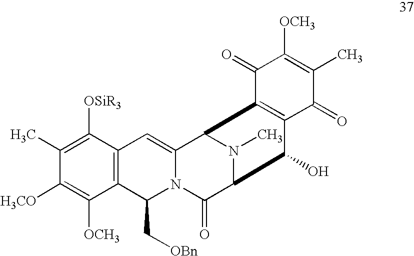

wherein each R is independently —C₁-C₁₂ alkyl or phenyl,
to react with an oxidizing agent under conditions that are sufficient to make the compound of formula 38.

The oxidizing agent is one that can oxidize a secondary alcohol to a ketone.

Examples of suitable oxidizing agents are well-known to those skilled in the art (M. B. Smith et al., March's Advanced Organic Chemistry: Reactions, Mechanisms and Structure 1514-1517 (5^{th} ed. 2001). In one embodiment, the oxidizing agent is pyridinium chlorochromate, pyridinium dichromate, potassium permanganate, manganese dioxide, Dess-Martin periodinane, ruthenium tetraoxide, tetra-n-propylammonium perruthenate, chromium trioxide, a combination of chromium trioxide and pyridine, Jones reagent, SO₃ in the presence of DMSO, N-chlorosuccinimide in the presence of dimethylsulfide and triethylamine (Corey-Kim oxidation), or oxalyl chloride in the presence of DMSO and a tertiary amine (Swem oxidation).

In a specific embodiment, the oxidizing agent is the Dess-Martin periodinane.

In one embodiment, about 1 to about 10 equivalents of oxidizing agent are used relative to about 1 equivalent of a compound of Formula 37.

In another embodiment, about 2 to about 5 equivalents of oxidizing agent are used relative to about 1 equivalent of a compound of Formula 37.

In a specific embodiment, about 1.5 equivalents of oxidizing agent are used relative to about 1 equivalent of a compound of Formula 37.

The method can be carried out in the presence of a reaction solvent, such as methylene chloride, chloroform, THF, acetone, ethyl acetate, or mixtures thereof.

In one embodiment, the solvent is methylene chloride.

In still another embodiment, the solvent is substantially anhydrous, i.e., comprises less than about 1% water.

In one embodiment, the method is carried out for a time of about 1 hour to about 36 hours.

In another embodiment, the method is carried out for a time of about 6 hours to about 24 hours,

In yet another embodiment, the method is carried out for a time of about 12 hours to about 18 hours.

In yet another embodiment, the method is carried out for a time of about 12 hours.

In one embodiment, the method is carried out at a temperature of about 0° C. to about 70° C.

In another embodiment, the method is carried out at a temperature of about 20° C. to about 40° C.

In still another embodiment, the method is carried out at a temperature of about 25° C.

Typically when the Dess-Martin periodinane is the oxidizing agent, the method is performed at room temperature in the presence of a reaction solvent.

In one embodiment, a mixture of a compound of formula 38 and a reaction solvent can be mixed with about 10% aqueous Na₂S₂O₃ and extracted with methylene chloride. The methylene chloride solution can be dried over a drying agent, filtered, and concentrated in vacuo to provide a crude product. The crude product can then be purified using a variety of chromatographic techniques, which are well-known to one skilled in the art of organic synthesis.

### 4.6 Methods For Making a Compound of Formula 39

In one embodiment, the invention provides a method for making a compound of formula 39:

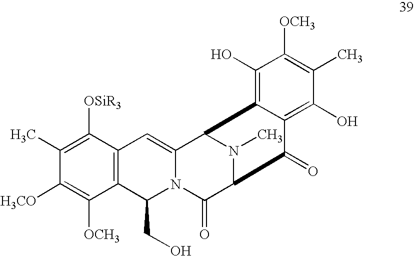

the method comprising allowing a compound of formula 38:

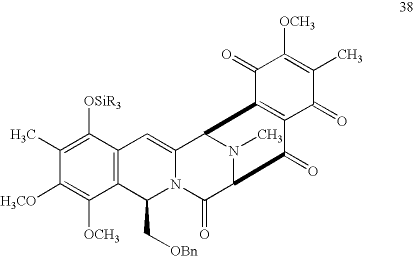

wherein each R is independently —C₁-C₁₂ alkyl or phenyl,
to react with H₂ in the presence of Pd/C under conditions that are sufficient to make the compound of formula 39.

In one embodiment the Pd/C has from about 1% palladium to about 20% palladium by weight of the Pd/C.

In another embodiment the Pd/C has from about 5% palladium to about 10% palladium by weight of the Pd/C.

In a specific embodiment, the Pd/C has about 10% palladium by weight of the Pd/C.

In one embodiment, the weight-to-weight ratio (w/w) of Pd/C to a compound of Formula 38 is from about 1% to about 20%.

In one embodiment, the weight-to-weight ratio (w/w) of Pd/C to a compound of Formula 38 is from about 5% to about 15%.

In one embodiment, the weight-to-weight ratio (w/w) of Pd/C to a compound of Formula 38 is about 10%.

The method can be carried out in the presence of a reaction solvent, such as ethyl acetate, methanol, ethanol, isopropanol, tert-butanol, benzene, toluene, THF or mixtures thereof.

In a specific embodiment, the solvent is methanol.

In another specific embodiment, the solvent is ethanol.

In still another embodiment, the solvent is substantially anhydrous, i.e., comprises less than about 1% water.

In one embodiment, the method is carried out for a time of about 1 hour to about 72 hours.

In another embodiment, the method is carried out for a time of about 12 hours to about 48 hours.

In yet another embodiment, the method is carried out for a time of about 18 hours to about 36 hours.

In yet another embodiment, the method is carried out for a time of about 24 hours.

In one embodiment, the method is carried out at a temperature of about 0° C. to about 60° C.

In another embodiment, the method is carried out at a temperature of about 10° C. to about 40° C.

In still another embodiment, the method is carried out at a temperature of about 25° C.

Typically H₂ gas is bubbled through a composition comprising a compound of formula 38, Pd/C and a reaction solvent. Alternatively, an atmosphere of H₂ is maintained over the composition.

In one embodiment an atmosphere of H₂ is maintained over the composition via a balloon containing H₂.

In one embodiment, a mixture of a compound of formula 39, Pd/C, and a reaction solvent can be filtered to remove Pd/C, then concentrated in vacuo.

### 4.7 Methods For Making a Compound of Formula 41

In one embodiment, the invention provides a method for making a compound of formula 41:

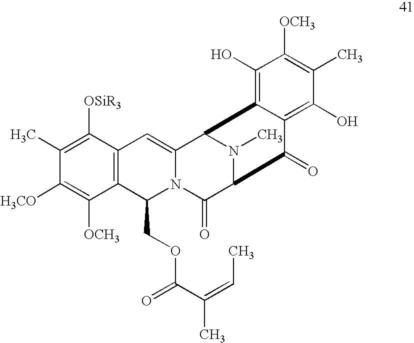

comprising allowing a compound of formula 39:

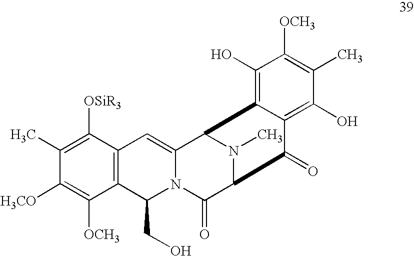

wherein each R is independently —C₁-C₁₂ alkyl or phenyl,
to react with a compound of formula 40:

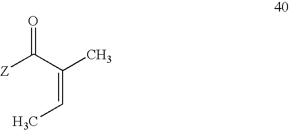

wherein Z is —Cl, —Br, —OH, —C(O)(C₁-C₁₂ alkyl), —C(O)-phenyl, or

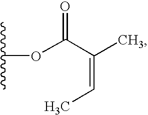

wherein phenyl is unsubstituted or substituted with up to 3 substituents independently selected from -halo, —C₁-C₁₂ alkyl, —O—(C₁-C₁₂ alkyl), —CN, —CF₃, or —NO₂, under conditions that are sufficient to make the compound of formula 41.

In one embodiment, Z is —Cl.

In another embodiment, Z is —Br.

In another embodiment, Z is —OH.

In still another embodiment, Z is —C(O)(C₁-C₁₂ alkyl).

In a further embodiment, Z is —C(O)-phenyl, wherein phenyl is unsubstituted or substituted with up to 3 substituents independently selected from -halo, —C₁-C₁₂ alkyl, —O—(C₁-C₁₂ alkyl), —CN, —CF₃, or —NO₂.

In one embodiment, when Z is —Cl, —Br, —C(O)(C₁-C₁₂ alkyl) or —C(O)-phenyl, the method can be performed in the presence of a base. Bases useful in the present methods include, but are not limited to amine bases, such as triethylamine, pyridine, 2,6-lutidine, and diisopropylethylamine; alkali metal hydrides, such as sodium hydride and potassium hydride; and alkali metal carbonates, such as sodium carbonate, potassium carbonate and cesium carbonate.

In another embodiment, when Z is —OH, the method can be performed in the presence of dicyclohexylcarbodiimide.

In one embodiment, about 1 to about 100 equivalents of Compound 40 are used relative to about 1 equivalent of a compound of Formula 39.

In another embodiment, about 10 to about 40 equivalents of Compound 40 are used relative to about 1 equivalent of a compound of Formula 39.

In another embodiment, about 20 to about 30 equivalents of Compound 40 are used relative to about 1 equivalent of a compound of Formula 39.

In another embodiment, about 25 equivalents of Compound 40 are used relative to about 1 equivalent of a compound of Formula 39.

The method can be carried out in the presence of a reaction solvent, such as methylene chloride, chloroform, THF, DMF, pyridine, acetone, acetonitrile, ethyl acetate, or mixtures thereof.

In one embodiment, the solvent is methylene chloride.

In still another embodiment, the solvent is substantially anhydrous, i.e., comprises less than about 1% water.

In one embodiment, the method is carried out for a time of about 1 hour to about 24 hours.

In another embodiment, the method is carried out for a time of about 6 hours to about 18 hours.

In yet another embodiment, the method is carried out for a time of about 12 hours.

In one embodiment, the method is carried out at a temperature of about 0° C. to about 60° C.

In another embodiment, the method is carried out at a temperature of about 10° C. to about 40° C.

In still another embodiment, the method is carried out at a temperature of about 25° C.

Typically, the method is performed by reacting a compound of formula 39 and a compound of formula 40 in a reaction solvent at room temperature.

In one embodiment, a mixture of a compound of formula 41 and a reaction solvent can be concentrated in vacuo.

### 4.8 Methods For Making Compound 42

In one embodiment, the invention provides a method for making Compound 42:

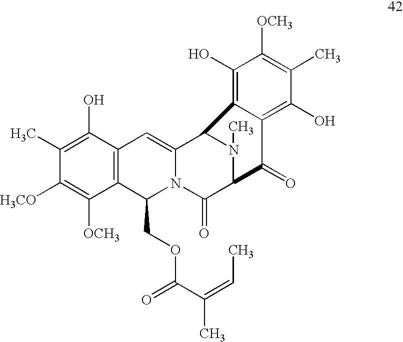

the method comprising allowing a compound of formula 41:

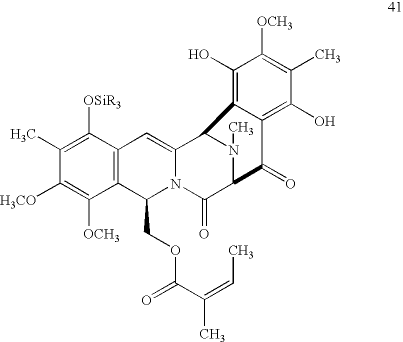

wherein each R is independently —C₁-C₁₂ alkyl or phenyl,
to react with a Bronsted acid or a fluoride salt under conditions that are sufficient to make Compound 42.

Fluoride salts useful in the present methods include tetra-n-butylammonium fluoride, potassium fluoride, sodium fluoride and cesium fluoride.

In one embodiment, the fluoride salt is tetra-n-butylammonium fluoride.

In one embodiment, the method comprises allowing a compound of formula 41 to react with a fluoride salt.

In another embodiment, the method comprises allowing a compound of formula 41 to react with a Bronsted acid.

In still another embodiment, the method further comprises allowing a compound of formula 41 to react with a fluoride salt in the presence of a Bronsted acid.

Bronsted acids useful in the present methods, include, but are not limited to, carboxylic acids, such as formic acid, acetic acid and trifluoromethyl acetic acid; and inorganic acids, such as hydrochloric acid, sulfuric acid, nitric acid, and hydrobromic acid.

In one embodiment, the Bronsted acid is a carboxylic acid.

In one embodiment, the Bronsted acid is an inorganic acid.

In a specific embodiment, the Bronsted acid is acetic acid.

In one embodiment, about 1 to about 10 equivalents of fluoride salt are used relative to about 1 equivalent of a compound of Formula 41.

In another embodiment, about 2 to about 5 equivalents of fluoride salt are used relative to about 1 equivalent of a compound of Formula 41.

In another embodiment, about 2.5 equivalents of fluoride salt are used relative to about 1 equivalent of a compound of formula 41.

In one embodiment, about 1 to about 50 equivalents of a Bronsted acid is used relative to about 1 equivalent of a compound of Formula 41.

In another embodiment, about 10 to about 40 equivalents of Bronsted acid is used relative to about 1 equivalent of a compound of Formula 41.

In another embodiment, about 15 to about 30 equivalents of Bronsted acid is used relative to about 1 equivalent of a compound of Formula 41.

In another embodiment, about 20 equivalents of Bronsted acid is used relative to about 1 equivalent of a compound of Formula 41.

In one embodiment, the method can be carried out in the presence of a reaction solvent, such as tetrahydrofuran, diethyl ether, water, or mixtures thereof.

In one embodiment, the solvent is tetrahydrofuran.

In still another embodiment, the solvent is substantially anhydrous, i.e., comprises less than about 1% water.

In one embodiment, the method is carried out for a time of about 0.2 hour to about 12 hours.

In another embodiment, the method is carried out for a time of about 0.5 hours to about 6 hours.

In yet another embodiment, the method is carried out for a time of about 1 hour to about 3 hours.

In yet another embodiment, the method is carried out for a time of about 2 hours.

In one embodiment, the method is carried out at a temperature of about −30° C. to about 25° C.

In another embodiment, the method is carried out at a temperature of about −10° C. to about 10° C.

In still another embodiment, the method is carried out at a temperature of about 0° C.

In a specific embodiment, the method is initially carried out at a temperature of about 0° C. and allowed to warm to about 25° C.

Typically, the method is performed by reacting a compound of formula 41 and fluoride salt in a reaction solvent at room temperature.

In one embodiment, a mixture of a compound of formula 42 and a reaction solvent can be concentrated in vacuo and purified using a variety of chromatographic techniques which are well-known to one skilled in the art of organic synthesis.

### 4.9 Methods For Making Compound 43

In one embodiment, the invention provides a method for making Compound 43:

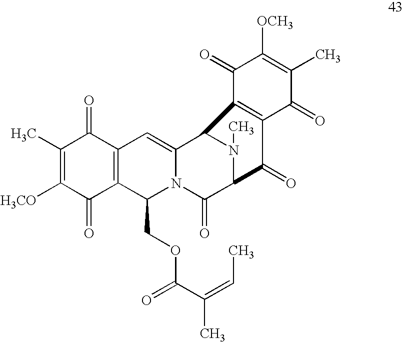

the method comprising allowing Compound 42:

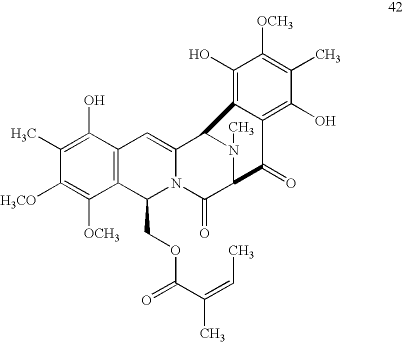

to react with (a) silver oxide, (b) phenyliodonium(III)diacetate, (c) ceric(IV) ammonium nitrate, (d) (KSO₃)₂NO, (e) PhSeCl, (f) MnO₂, (g) phenyliodine (III) bis(trifluoroacetate), or (h) O₂ in the presence of a metal, under conditions that are sufficient to make Compound 43.

In another embodiment, Compound 42 is allowed to react with silver oxide.

In another embodiment, Compound 42 is allowed to react with phenyliodonium(III) diacetate.

In still another embodiment, Compound 42 is allowed to react with ceric(IV) ammonium nitrate.

In yet another embodiment, Compound 42 is allowed to react with (KSO₃)₂NO.

In a further embodiment, Compound 42 is allowed to react with PhSeCl.

In another embodiment, Compound 42 is allowed to react with MnO₂.

In another embodiment, Compound 42 is allowed to react with phenyliodine(III)bis(trifluoroacetate).

In yet another embodiment, Compound 42 is allowed to react with O₂ in the presence of a metal.

Metals useful in the present methods include, but are not limited to copper(I), copper (II), iron (II), manganese (II), manganese (VII), and cobalt (III).

In one embodiment, the compound of formula 42 is allowed to react in the presence of phenyliodine(III)bis(trifluoroacetate).

In another embodiment, the compound of formula 42 is allowed to react in the presence of silver oxide.

In one embodiment, about 1 to about 10 equivalents of a reagent is used relative to about 1 equivalent of a compound of Formula 42.

In another embodiment, about 2 to about 5 equivalents of a reagent is used relative to about 1 equivalent of a compound of Formula 42.

In a specific embodiment, about 2.5 equivalents of a reagent is used relative to about 1 equivalent of a compound of Formula 42.

The method can be carried out in the presence of a reaction solvent, such as acetonitrile, ether, THF, an organic alcohol, acetone, toluene, benzene, methylene chloride, chloroform, water, or mixtures thereof.

In one embodiment, when the oxidizing agent of Compound 42 is phenyliodine (III) bis(trifluoroacetate), the solvent is a mixture of acetonitrile and water.

In a specific embodiment, when the oxidizing agent of Compound 42 is phenyliodine(III)bis(trifluoroacetate), the solvent is a 2:1 mixture of acetonitrile:water.

In still another embodiment, the solvent is substantially anhydrous, i.e., comprises less than about 1% water.

In one embodiment, the method is carried out for a time of about 1 minute to about 1 hour.

In another embodiment, the method is carried out for a time of about 5 minutes to about 30 minutes.

In yet another embodiment, the method is carried out for a time of about 10 minutes to about 20 minutes.

In yet another embodiment, the method is carried out for a time of about 10 minutes.

In one embodiment, the method is carried out at a temperature of about 0° C. to about 70° C.

In another embodiment, the method is carried out at a temperature of about 20° C. to about 40° C.

In still another embodiment, the method is carried out at a temperature of about 25° C.

In one embodiment, a mixture of a compound of formula 43 and a reaction solvent is mixed with saturated aqueous NaHCO₃ and extracted with methylene chloride. The methylene chloride solution can be dried over a drying agent, filtered, and concentrated in vacuo.

### 4.10 Methods For Making Compound 44

In one embodiment, the invention provides a method for making Compound 44:

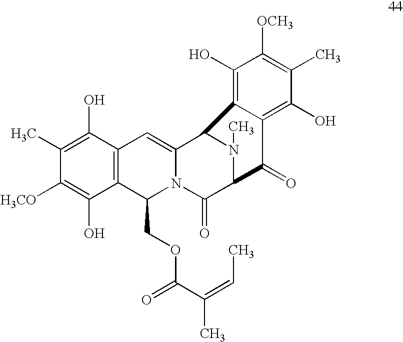

the method comprising allowing Compound 43:

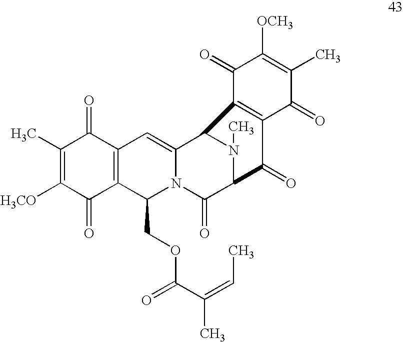

to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44.

In one embodiment, Compound 43 is allowed to react with Na₂S₂O₃.

In another embodiment, Compound 43 is allowed to react with NaHSO₃.

In another embodiment, Compound 43 is allowed to react with NaBH₄.

In still another embodiment, Compound 43 is allowed to react with Na₂(SO₂)₂.

In yet another embodiment, Compound 43 is allowed to react with a mixture of zinc metal and a Bronsted acid.

In a specific embodiment, the zinc metal is in the form of zinc dust.

Bronsted acids useful in the present methods, include, but are not limited to, carboxylic acids, such as formic acid, acetic acid and trifluoromethyl acetic acid; and inorganic acids, such as hydrochloric acid, sulfuric acid, nitric acid, and hydrobromic acid.

In one embodiment, the Bronsted acid is a carboxylic acid.

In one embodiment, the Bronsted acid is an inorganic acid.

In a specific embodiment, the Bronsted acid is acetic acid.

In one embodiment, about 1 to about 100 equivalents of a reagent is used relative to about 1 equivalent of a compound of Formula 43.

In another embodiment, about 10 to about 50 equivalents of a reagent is used used relative to about 1 equivalent of a compound of Formula 43.

In another embodiment, about 20 to about 30 equivalents of a reagent is used relative to about 1 equivalent of a compound of Formula 43.

In a specific embodiment, about 20 equivalents of a reagent is used relative to about 1 equivalent of a compound of Formula 43.

In one embodiment, the method is carried out for a time of about 1 minute to about 1 hour.

In another embodiment, the method is carried out for a time of about 5 minutes to about 30 minutes.

In yet another embodiment, the method is carried out for a time of about 10 minutes to about 20 minutes.

In yet another embodiment, the method is carried out for a time of about 10 minutes.

In one embodiment, the method is carried out at a temperature of about 0° C. to about 70° C.

In another embodiment, the method is carried out at a temperature of about 20° C. to about 40° C.

In still another embodiment, the method is carried out at a temperature of about 25° C.

In one embodiment, a mixture of a compound of formula 44 and a reaction solvent can be mixed with saturated aqueous NaHCO₃ and extracted with methylene chloride. The methylene chloride solution can be dried over a drying agent, filtered, and concentrated in vacuo.

### 4.11 Methods For Making Cribrostatin IV from Compound 44

In one embodiment, the invention provides a method for making Cribrostatin IV:

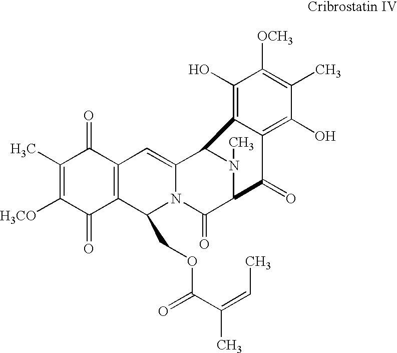

from Compound 44, the method comprising allowing Compound 44:

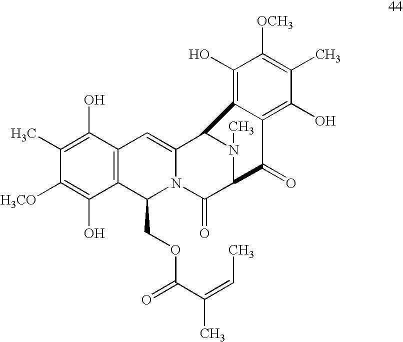

to react with O₂ under conditions that are sufficient to make Cribrostatin IV.

The method can be carried out in the presence of a reaction solvent, such as TB-F, DMF, acetone, acetonitrile, methylene chloride, chloroform, benzene, toluene, acetone, ethyl acetate, water, or mixtures thereof.

In one embodiment, the solvent is DMF.

In still another embodiment, the solvent is substantially anhydrous, i.e., comprises less than about 1% water.

In one embodiment, O₂ is introduced into the reaction solvent by bubbling substantially pure O₂ through a mixture of Compound 44 and a reaction solvent.

In one embodiment, O₂ is introduced into the reaction solvent by bubbling air through a mixture of Compound 44 and a reaction solvent.

In one embodiment, O₂ is bubbled through a mixture of Compound 44 and a reaction solvent for a period of about 1 minute to about 20 minutes.

In another embodiment, O₂ is bubbled through a mixture of Compound 44 and a reaction solvent for a period of about 5 minute to about 10 minutes.

In a specific embodiment, O₂ is bubbled through a mixture of Compound 44 and a reaction solvent for a period of about 5 minutes.

In a specific embodiment, the method is carried out for a time of about 1 minute to about 1 hour.

In another embodiment, the method is carried out for a time of about 5 minutes to about 30 minutes.

In yet another embodiment, the method is carried out for a time of about 10 minutes to about 20 minutes.

In yet another embodiment, the method is carried out for a time of about 10 minutes.

In one embodiment, the method is carried out at a temperature of about 0° C. to about 70° C.

In another embodiment, the method is carried out at a temperature of about 20° C. to about 40° C.

In still another embodiment, the method is carried out at a temperature of about 25° C.

Typically, the method is performed by reacting a compound of formula 44 with O₂ in the presence of a reaction solvent.

In one embodiment, O₂ is introduced into the reaction by bubbling air through a solution comprising Compound 44 and a reaction solvent.

In one embodiment, a mixture of Cribrostatin IV and a reaction solvent is concentrated in vacuo and purified using a variety of chromatographic techniques that are well-known to one skilled in the art of organic synthesis.

### 4.12 Methods For Making Cribrostatin IV From a Compound of Formula 34

In a particular embodiment, the present invention provides a method for making Cribrostatin IV:

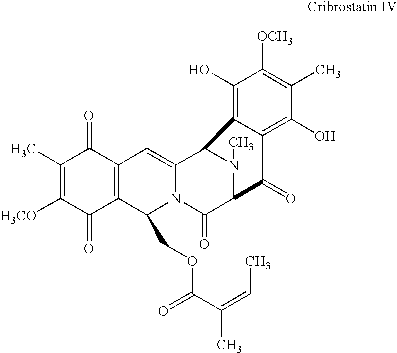

the method comprising the steps:
(i) allowing a compound of formula 34:

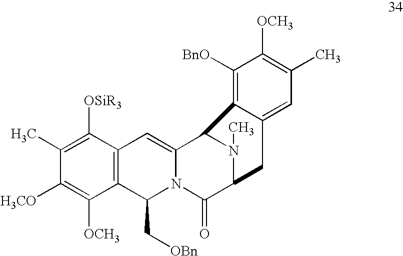

wherein each R is independently —C₁-C₁₂ alkyl or phenyl,
to react with H₂ in the presence of Pd/C under conditions that are sufficient to make a compound of formula 35:

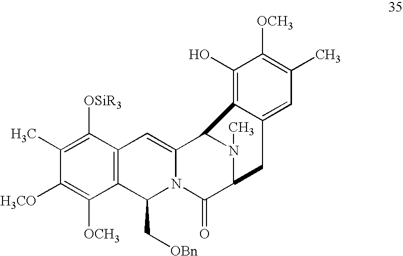

(ii) allowing a compound of formula 35 to react with (a) (KSO₃)₂NO, (b) DDQ, or (c) O₂ in the presence of a metal, under conditions that are sufficient to make a compound of formula 36:

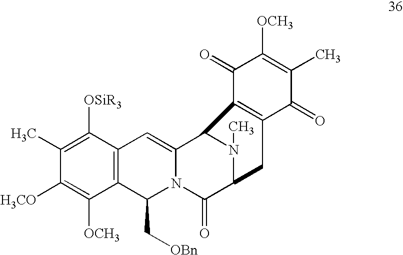

(iii) allowing a compound of formula 36 to react with SeO₂ under conditions that are sufficient to make a compound of formula 37:

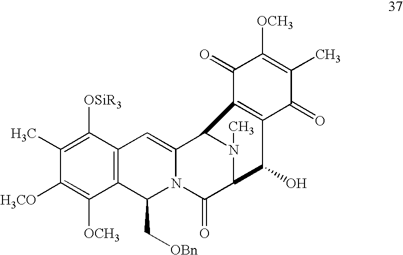

(iv) allowing a compound of formula 37 to react with an oxidizing agent under conditions that are sufficient to make a compound of formula 38:

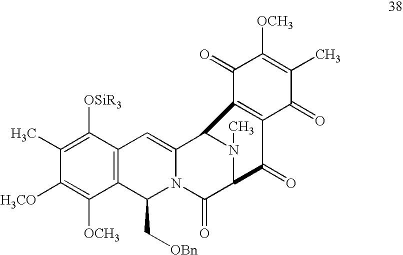

(v) allowing a compound of formula 38 to react with H₂ in the presence of Pd/C under conditions that are sufficient to make a compound of formula 39:

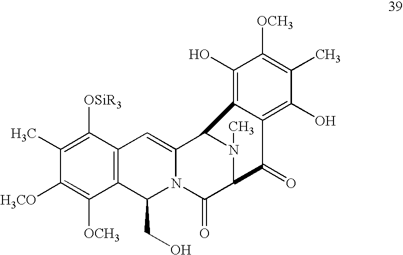

(vi) allowing a compound of formula 39 to react with a compound of formula 40:

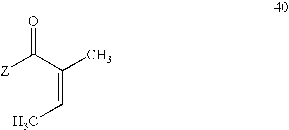

wherein Z is —Cl, —Br, —OH, —C(O)(C₁-C₁₂ alkyl), —C(O)-phenyl, or

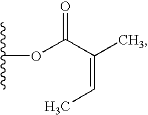

wherein phenyl is unsubstituted or substituted with up to 3 substituents independently selected from -halo, —C₁-C₁₂ alkyl, —O—(C₁-C₁₂ alkyl), —CN, —CF₃, or —NO₂, under conditions that are sufficient to make a compound of formula 41:

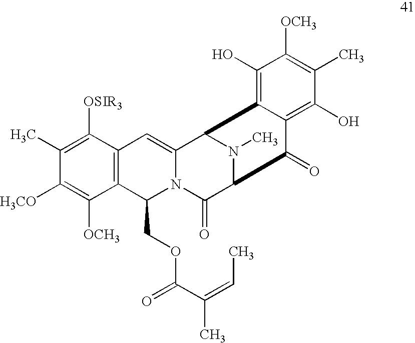

(vii) allowing a compound of formula 41 to react with a fluoride salt under conditions that are sufficient to make Compound 42:

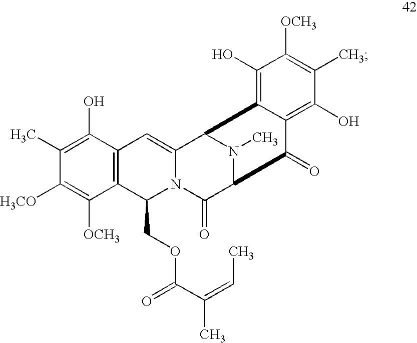

(viii) allowing Compound 42 to react with (a) silver oxide, (b) phenyliodonium(III) diacetate, (c) ceric(IV) ammonium nitrate, (d) (KSO₃)₂NO, (e) PhSeCl, (f) MnO₂, (g) phenyliodine (III) bis(trifluoroacetate), or (h) O₂ in the presence of a metal, under conditions that are sufficient to make Compound 43:

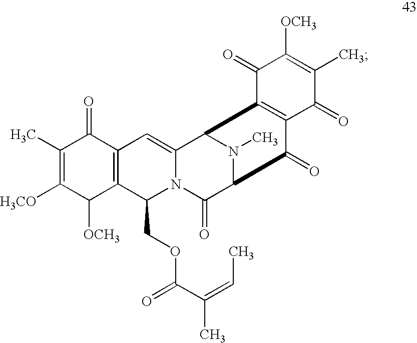

(ix) allowing Compound 43 to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44:

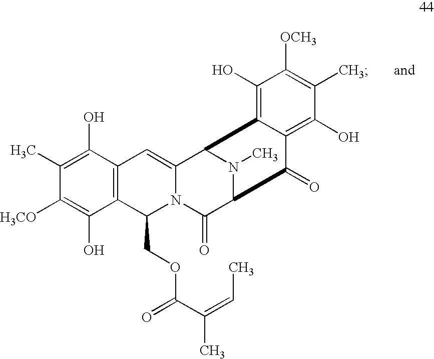

(x) allowing Compound 44 to react with O₂ under conditions that are sufficient to make Cribrostatin IV.

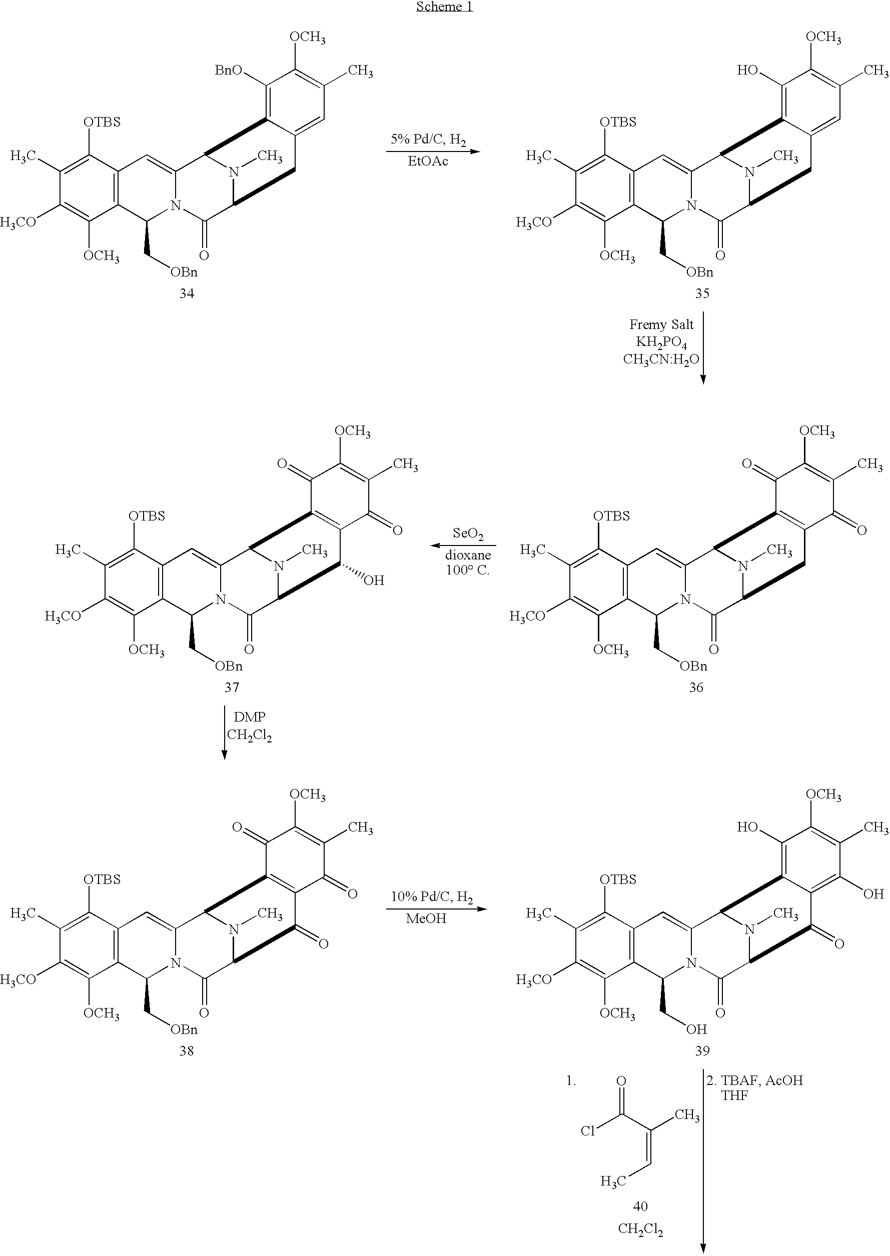

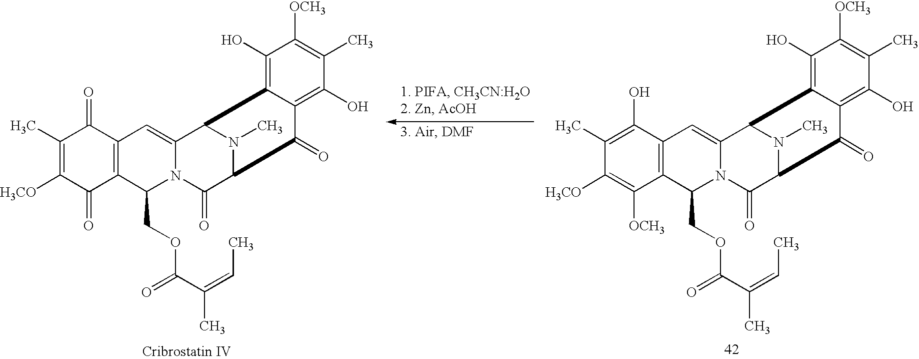

### 4.13 Methods for Making Compound 34

Compound 34, which is useful as a starting material for making Cribrostatin IV, can be made according to the method set forth in Scheme 2 using methods and techniques well-known to one of skill in the art of organic synthesis.

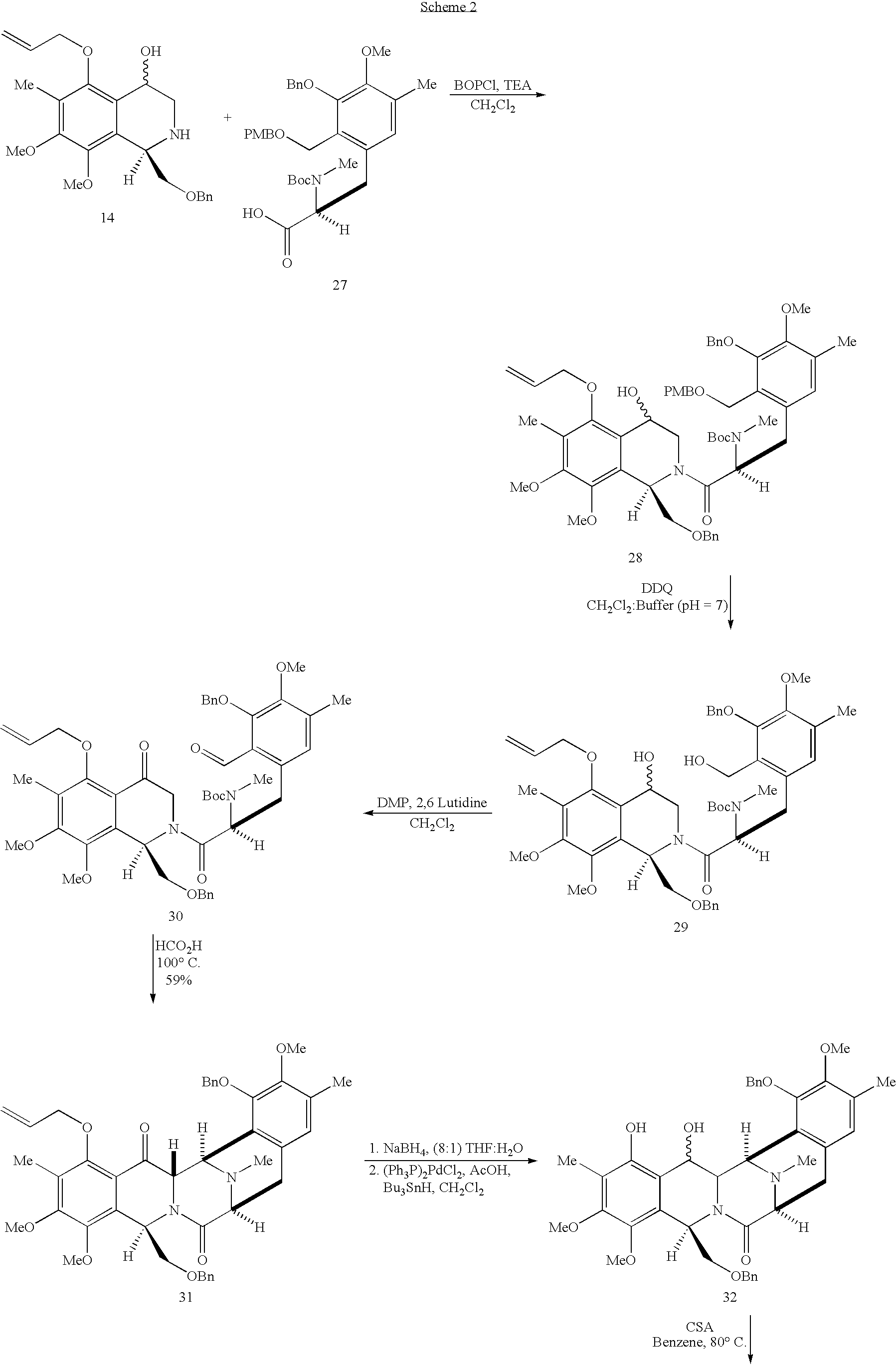

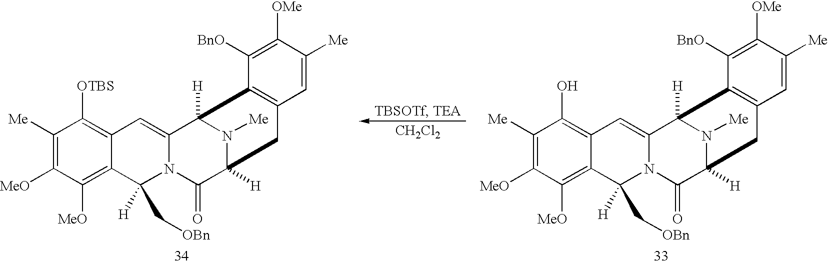

Isoquinoline Compound 14 is coupled with carboxylic acid 27 in the presence of BOPCl to provide amido intermediate 28. The PMB protecting benzyl alcohol of 28 is then removed using DDQ to provide hydroxymethyl Compound 29, which is subsequently oxidized using DMP in the presence of 2,6-lutidine to provide aldehyde intermediate 30. Removal of the BOC protecting group of 30 using formic acid reveals the N-methyl amino group which subsequently undergoes an internal cyclization with the aldehyde functionality of 30 to provide pentacyclic Compound 31. Reduction of the keto group of 31 using NaBH₄, followed by deprotection of the allyl-protected phenol using a palladium/tin mediated process, affords Compound 32. Treatment of 32 with CSA results in elimination of the aliphatic hydroxyl group to provide olefin intermediate 33, the phenol group of which is subsequently protected as its TBS ether to provide Compound 34.

Compound 14, which is a useful starting material for making pentacyclic Compound 34, can be made according to the method set forth in Scheme 3 using methods and techniques well-known to one of skill in the art of organic synthesis.

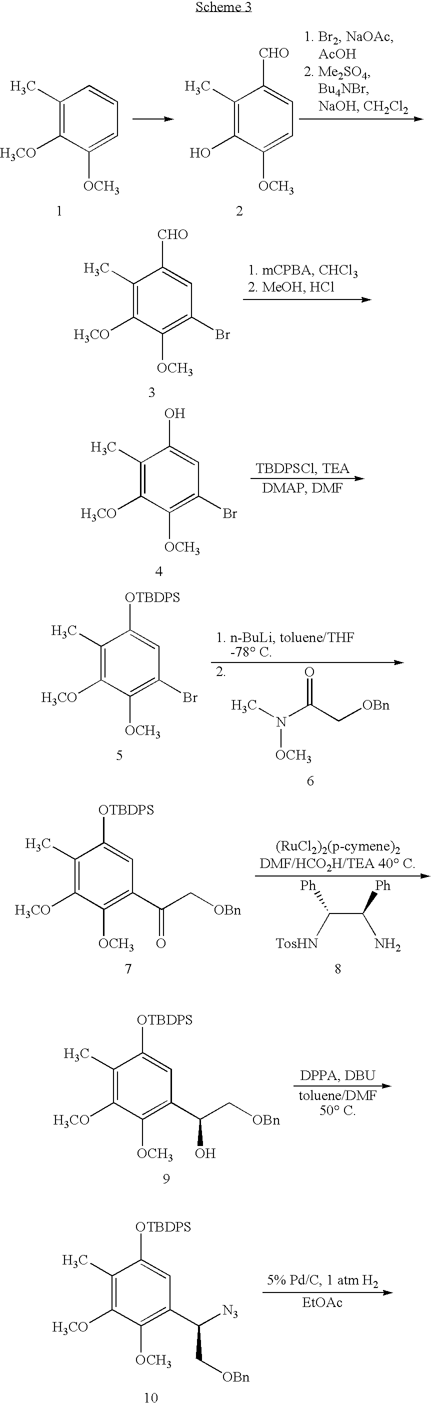

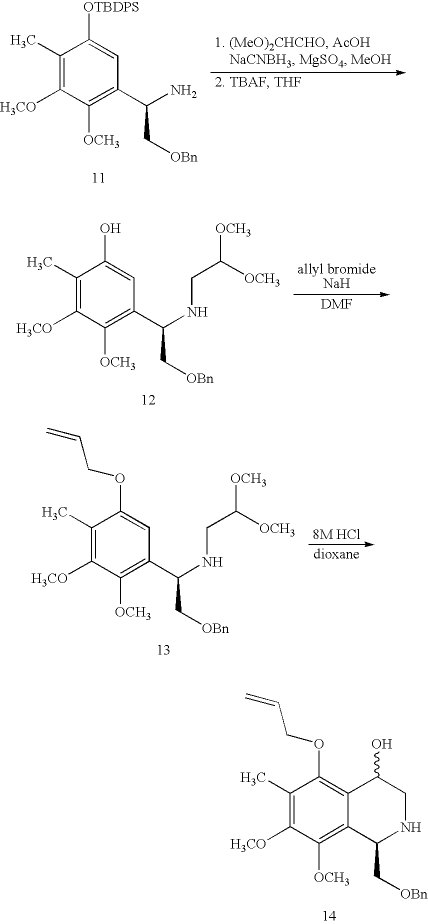

1,2-dimethoxy-3-methyl benzene 1 can be formylated using methods described in Sinhababu et al., J. Med. Chem. 28:1273 (1985) to provide benzaldehyde Compound 2. Compound 2 is then brominated using Br₂, followed by methylation of the two hydroxy groups using dimethylsulfate to provide bromo Compound 3. Baeyer-Villiger oxidation of 3 using mCPBA, followed by acid-catalyzed hydrolysis of the resultant ester provides phenol intermediate 4. Protection of phenol 4 as its TBDPS ether 5, followed by treatment of 5 with n-BuLi and reaction of the resultant phenyllithium intermediate with 2-benzyloxy-N-methoxy-N-methyl acetamide (6) affords phenyl ketone intermediate 7. Asymmetric transfer hydrogenation of the keto group of 7 using Ru-TsDPEN catalyst in the presence of chiral auxiliary 8, provides β-hydroxy Compound 9. Treatment of 9 with DPPA in the presence of DBU provides azide 10, which is subsequently reduced using catalytic hydrogenation to provide amine 11. The amino group of Compound 11 is coupled with dimethoxy acetaldehyde, and the resultant dimethoxy ethylamine intermediate is treated with tetra-n-butylammonium fluoride to remove the TBDPS protecting group and afford dimethyl acetal Compound 12. Reaction of 12 with allyl bromide in the presence of NaH provides allyl phenyl ether 13, which is subsequently cyclized in the presence of acid to provide Compound 14.

Compound 27, which is a useful starting material for making pentacyclic Compound 34, can be made according to the method set forth in Scheme 4 using methods and techniques well-known to one of skill in the art of organic synthesis.

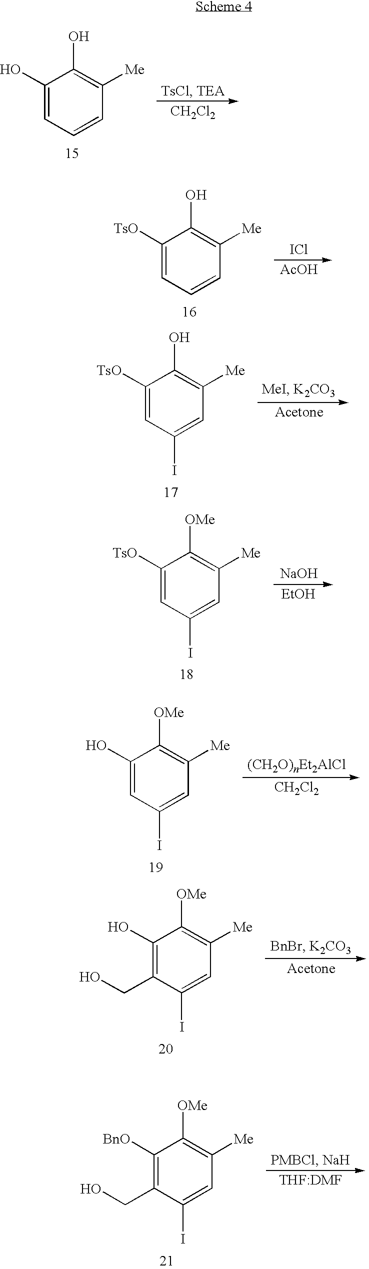

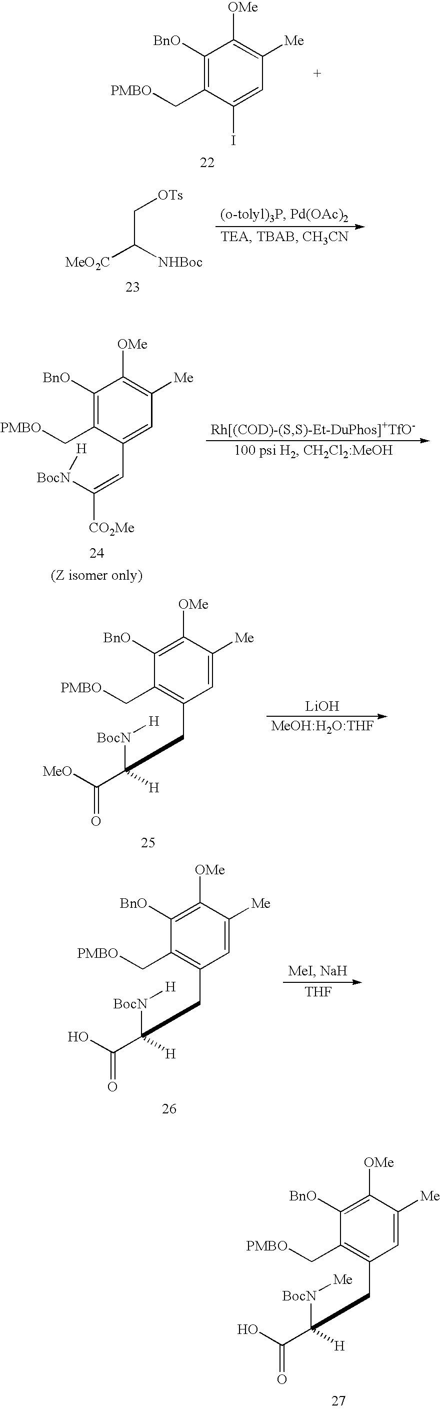

1,2-dihydroxytoluene 15 can be tosyllated using tosyl chloride in the presence of an amine base, such as triethylamine, to provide the monotosylated phenyl Compound 16. Iodination of 16 using ICl provides iodobenzene 17, which can be reacted with excess methyl iodide in the presence of a base to provide methoxyphenyl Compound 18. Removal of the tosyl group from 18 under basic hydrolysis conditions provides phenol 19, which is then subjected to an ortho-formylation using formaldehyde to provide hydroxymethyl Compound 20. Selective benzylation of the phenolic hydroxy group using benzyl bromide and potassium carbonate provides benzyl Compound 21, which is reacted with PMBCl and sodium hydride to protect the aliphatic hydroxy group of 21 as its PMB ester, providing Compound 22. Compound 22 is then reacted with ester 23 in a Jeffery-Heck coupling procedure to provide stryene Compound 24 as its Z-isomer. The olefin of 24 can then be enantiotopically reduced using Rh[(COD)-(S,S)-Et-DuPhos]⁺TfO⁻ at elevated pressure to provide ester 25. Base-catalyzed hydrolysis of 25 provides carboxylic acid 26 which can be reacted with methyl iodide to provide the N-Boc intermediate 27.

### 4.14 The Compounds of Formulas 34, 35, 36, 37, 38, 39, 41, 42, 43 and 44

In one aspect, the present invention provides a compound of formula 34, 35, 36, 37, 38, 39, 41, 42, 43 or 44, as depicted above herein. The compounds of formulas 34, 35, 36, 37, 38, 39, 41, 42, 43 and 44 are useful as starting materials or intermediates for making Cribrostatin IV.

### 4.15 Pentacyclic Alkaloid Compounds of Formula (I)

As stated above, the present invention provides Pentacyclic Alkaloid Compounds of Formula (I):

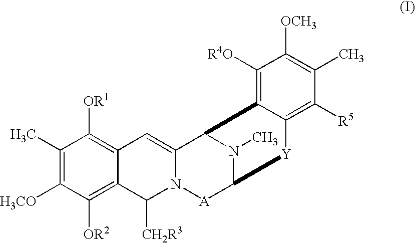

and pharmaceutically acceptable salts thereof,
where R¹, R², R³, R⁴, R⁵, A and Y are defined above for the Pentacyclic Alkaloid Compounds of Formula (I).

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R¹ is allyl.

In another embodiment R¹ is —H.

In still another embodiment R¹ is —C₁-C₁₂ alkyl.

In yet another embodiment R¹ is —C(O)-aryl.

In a specific embodiment R¹ is —C(O)—C₁-C₁₂ alkyl.

In a further embodiment R¹ is —C(O)—CH₃.

In an even further embodiment R¹ is —SO₂CH₃.

In one embodiment R² is —H.

In another embodiment R² is —C₁-C₁₂ alkyl.

In one embodiment R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

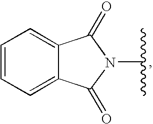

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In a specific embodiment R³ is —OC(O)—C₁-C₁₂ alkyl.

In yet another embodiment R³ is —OC(O)—CH₃.

In still another embodiment R³ is —NHC(O)—C₁-C₁₂ alkyl.

In a specific embodiment R³ is —NHC(O)—CH₃.

In another embodiment R³ is —NHC(O)-aryl.

In yet another embodiment R³ is —NHC(O)—C(O)—C₁-C₁₂ alkyl.

In a further embodiment R³ is —C(O)—NH—C(O)—C₁-C₁₂ alkyl.

In still another embodiment R³ is —O-aryl.

In a specific embodiment R³ is —O-phenyl.

In yet another embodiment R³ is:

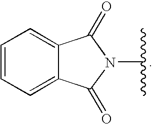

In one embodiment, the —CH₂R³ is in the α-configuration, i.e., below the plane, of the Pentacyclic Alkaloid Compound of formula (I) as depicted.

In another embodiment, the —CH₂R³ is in the β-configuration, i.e., above the plane, of the Pentacyclic Alkaloid Compound of formula (I) as depicted.

In one embodiment R⁴ is —H or —C₁-C₁₂ alkyl.

In another embodiment R⁴ is —H.

In yet another embodiment R⁴ is —C₁-C₁₂ alkyl.

In a specific embodiment R⁴ is -benzyl.

In one embodiment R⁵ is —H.

In another embodiment R⁵ is —OH.

In still another embodiment R⁵ is —O—C₁-C₁₂ alkyl.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In another embodiment R¹ and R² are each —H.

In another embodiment R¹ and R² are each —H, and A is —C(O)—.

In still another embodiment R¹ and R² are each —H, and A and Y are each —C(O)—.

In one embodiment R⁴ is —H, and R⁵ is —OH.

In one embodiment, R¹, R² and R⁴ are each —H.

In another embodiment, R¹, R² and R⁴ are each —H; A is —CH(α-OH)—; and Y is —C(O)—.

In yet another embodiment, R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

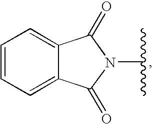

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R⁴ is —H or —C₁-C₁₂ alkyl.

In one embodiment, the compound of formula (I) has the formula (Ia):

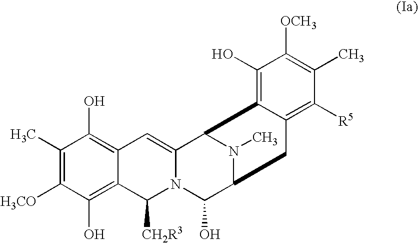

wherein R³ and R⁵ are defined above for the compounds of formula (I).

In another embodiment, the compound of formula (I) has the formula (Ib):

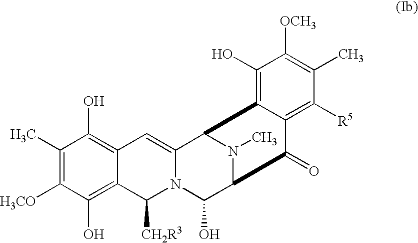

wherein R³ and R⁵ are defined above for the compounds of formula (I).

Illustrative compounds of formula (I) include the following:

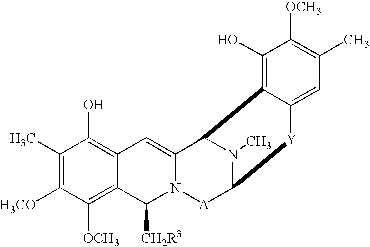

| Compound | A | Y | R³ |
|---|---|---|---|
| A | —CH(α-OH)— | —C(O)— | —OC(O)-n-propyl |
| B | —CH(α-OH)— | —C(O)— | —NHC(O)C(O)CH₃ |
| C | —CH(α-CN)— | —C(O)— | —OC(O)-n-propyl |
| D | —CH(α-CN)— | —C(O)— | —NHC(O)C(O)CH₃ |
| E | —C(O)— | —C(O)— | —OC(O)-n-propyl |
| F | —C(O)— | —C(O)— | —NHC(O)C(O)CH₃ |
| G | —CH(α-OH)— | —CH₂— | —OC(O)-n-propyl |
| H | —CH(α-OH)— | —CH₂— | —NHC(O)C(O)CH₃ |
| I | —CH(α-CN)— | —CH₂— | —OC(O)-n-propyl |
| J | —CH(α-CN)— | —CH₂— | —NHC(O)C(O)CH₃ |
| K | —C(O)— | —CH₂— | —OC(O)-n-propyl |
| L | —C(O)— | —CH₂— | —NHC(O)C(O)CH₃ |
| DZ | —CH(α-OH)— | —C(O)— | —O-benzyl |
| EA | —CH(α-CN)— | —C(O)— | —O-benzyl |
| EB | —C(O)— | —C(O)— | —O-benzyl |
| EC | —CH(α-OH)— | —CH₂— | —O-benzyl |
| ED | —CH(α-CN)— | —CH₂— | —O-benzyl |
| EF | —C(O)— | —CH₂— | —O-benzyl |

Additional illustrative compounds of formula (I) include the following:

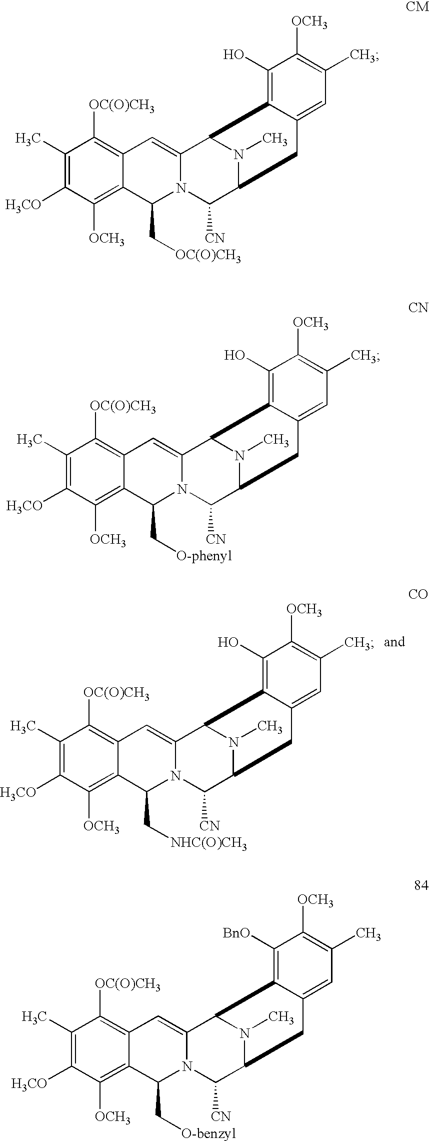

and pharmaceutically acceptable salts thereof.

### 4.16 Pentacyclic Alkaloid Compounds of Formula (II)

As stated above, the present invention provides Pentacyclic Alkaloid Compounds of Formula (II):

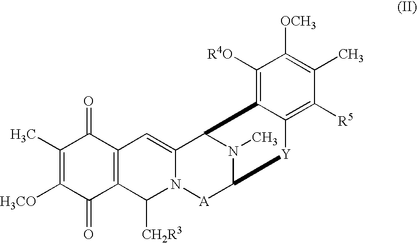

and pharmaceutically acceptable salts thereof,
where R³, R⁴, R⁵, A and Y are defined above for the Pentacyclic Alkaloid Compounds of Formula (II).

In one embodiment R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl or

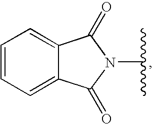

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In yet another embodiment R³ is —OC(O)—C₁-C₁₂ alkyl.

In a specific embodiment R³ is —OC(O)—CH₃.

In another embodiment R³ is —NHC(O)—C₁-C₁₂ alkyl.

In still another embodiment R³ is —NHC(O)-aryl.

In a further embodiment R³ is -(O)—NH—C(O)—C₁-C₁₂ alkyl.

In still another embodiment R³ is —O-aryl.

In a specific embodiment R³ is —O-phenyl.

In yet another embodiment R³ is:

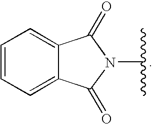

In one embodiment, the —CH₂R³ is in the α-configuration, i.e., below the plane, of the Pentacyclic Alkaloid Compound of formula (II) as depicted.

In another embodiment, the —CH₂R³ is in the α-configuration, i.e., above the plane, of the Pentacyclic Alkaloid Compound of formula (II) as depicted.

In one embodiment R⁴ is —H or —C₁-C₁₂ alkyl.

In a specific embodiment R⁴ is -benzyl.

In another embodiment R⁴ is —H.

In yet another embodiment R⁴ is C₁-C₁₂ alkyl.

In one embodiment R⁴ is —H.

In another embodiment R⁵ is —OH.

In still another embodiment R⁵ is —O—C₁-C₁₂ alkyl.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment-Y is —CH(α-OH)—.

In still another embodiment Y is —C(O)—.

In one embodiment A and Y are each —C(O)—.

In one embodiment R⁴ is —H, and R¹ is —OH.

In one embodiment, R⁴ is —H.

In another embodiment, R⁴ is —H; A is —CH(α-OH)—; and Y is —C(O)—.

In yet another embodiment, R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl or

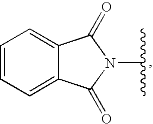

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R⁴ is —H or —C₁-C₁₂ alkyl.

In one embodiment, the compound of formula (II) has the formula (IIa):

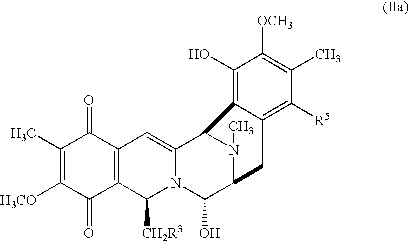

wherein R³ and R¹ are defined above for the compounds of formula (II).

In another embodiment, the compound of formula (II) has the formula (IIb):

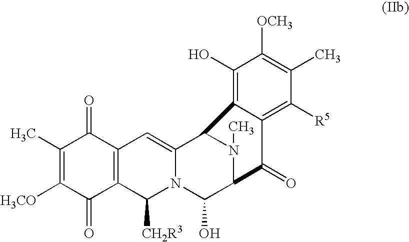

wherein R³ and R⁵ are defined above for the compounds of formula (II).

Illustrative compounds of formula (II) include the following:

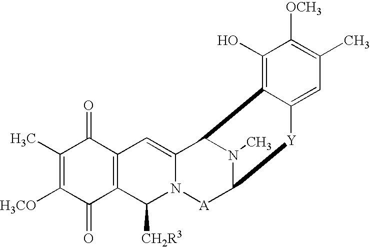

| Compound | A | Y | R³ |
|---|---|---|---|
| M | —CH(α-OH)— | —C(O)— | —OC(O)-n-propyl |
| N | —CH(α-OH)— | —C(O)— | —NHC(O)CH₃ |
| O | —CH(α-CN)— | —C(O)— | —OC(O)-n-propyl |
| P | —CH(α-CN)— | —C(O)— | —NHC(O)CH₃ |
| Q | —C(O)— | —C(O)— | —OC(O)-n-propyl |
| R | —C(O)— | —C(O)— | —NHC(O)CH₃ |
| S | —CH(α-OH)— | —CH₂— | —OC(O)-n-propyl |
| T | —CH(α-OH)— | —CH₂— | —NHC(O)CH₃ |
| U | —CH(α-CN)— | —CH₂— | —OC(O)-n-propyl |
| V | —CH(α-CN)— | —CH₂— | —NHC(O)CH₃ |
| W | —C(O)— | —CH₂— | —OC(O)-n-propyl |
| X | —C(O)— | —CH₂— | —NHC(O)CH₃ |
| EG | —CH(α-OH)— | —C(O)— | —O-benzyl |
| EH | —CH(α-CN)— | —C(O)— | —O-benzyl |
| EI | —C(O)— | —C(O)— | —O-benzyl |
| EJ | —CH(α-OH)— | —CH₂— | —O-benzyl |
| EK | —CH(α-CN)— | —CH₂— | —O-benzyl |
| EL | —C(O)— | —CH₂— | —O-benzyl |

Additional illustrative compounds of formula (II) include the following:

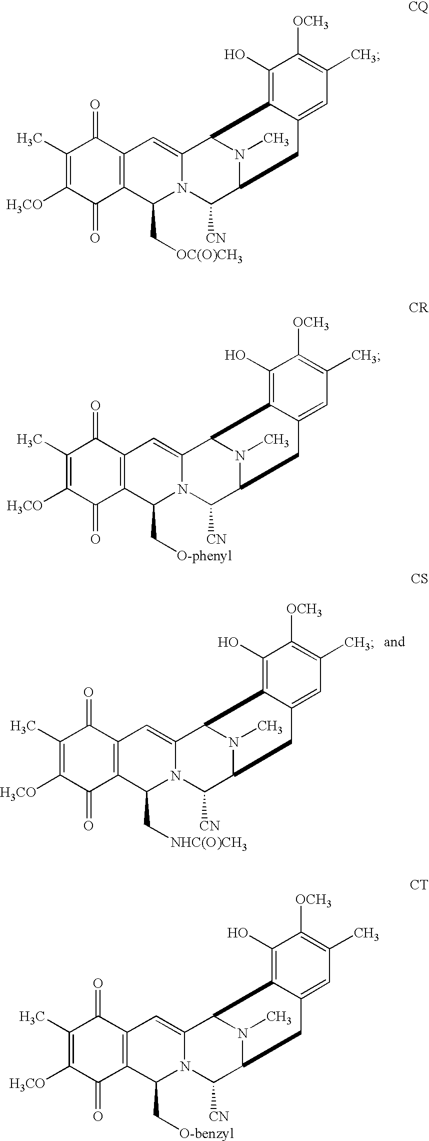

and pharmaceutically acceptable salts thereof

### 4.17 Pentacyclic Alkaloid Compounds of Formula (III)

As stated above, the present invention provides Pentacyclic Alkaloid Compounds of Formula (III):

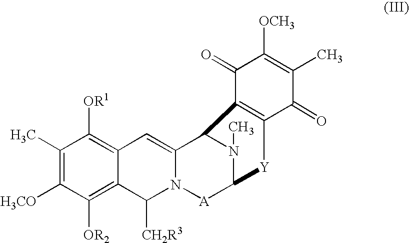

and pharmaceutically acceptable salts thereof,
where R′, R¹, R³, A and Y are defined above for the Pentacyclic Alkaloid Compounds of Formula (III).

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R¹ is -allyl.

In yet another embodiment R¹ is —H.

In a specific embodiment R¹ is —C₁-C₂ alkyl.

In still another embodiment R¹ is —C(O)-aryl.

In yet another embodiment R¹ is —C(O)—C₁-C₁₂ alkyl.

In a specific embodiment R¹ is —C(O)—CH₃.

In a further embodiment R¹ is —SO₂CH₃.

In one embodiment R² is —H.

In another embodiment R² is —C₁-C₁₂ alkyl.

In one embodiment R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

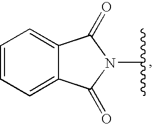

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In yet another embodiment R³ is —OC(O)—C₁-C₁₂ alkyl.

In a specific embodiment R³ is —OC(O)—CH₃.

In another embodiment R³ is —NHC(O)—C₁-C₁₂ alkyl.

In still another embodiment R³ is —NHC(O)-aryl.

In yet another embodiment R³ is —NHC(O)—C(O)—C₁-C₁₂ alkyl.

In a further embodiment R³ is —C(O)—NH—C(O)—C₁-C₁₂ alkyl.

In still another embodiment R³ is —O-aryl.

In a specific embodiment R³ is —O-phenyl.

In yet another embodiment R³ is:

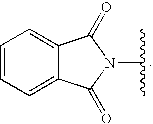

In one embodiment, the —CH₂R³ is in the α-configuration, i.e., below the plane of the Pentacyclic Alkaloid Compound of formula (III) as depicted.

In another embodiment, the —CH₂R³ is in the β-configuration, i.e., above the plane of the Pentacyclic Alkaloid Compound of formula (III) as depicted.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In yet another embodiment A and Y are each —C(O)—.

In another embodiment R¹ and R² are each —H.

In another embodiment R¹ and R² are each —H, and A is —C(O)—.

In still another embodiment R¹ and R² are each —H, and A and Y are each —C(O)—.

In another embodiment, R¹ and R² are each —H; A is —CH(α-OH)—; and Y is —C(O)—.

In yet another embodiment, R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

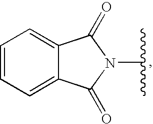

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In one embodiment, the compound of formula (III) has the formula (IIIa):

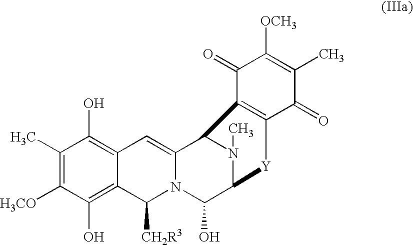

wherein R³ is defined above for the compounds of formula (III).

In another embodiment, the compound of formula (III) has the formula (IIIb):

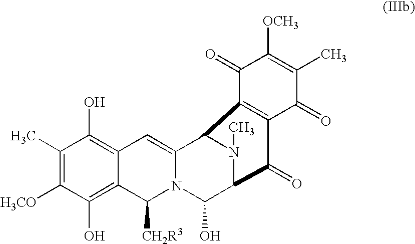

wherein R³ is defined above for the compounds of formula (III).

Illustrative compounds of formula (III) include the following:

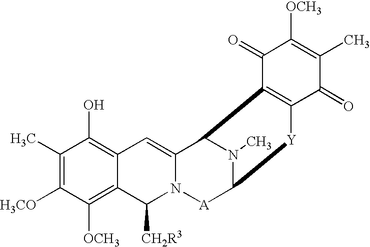

| Compound | A | Y | R³ |
|---|---|---|---|
| Y | —CH(α-OH)— | —C(O)— | —OC(O)-n-propyl |
| Z | —CH(α-OH)— | —C(O)— | —NHC(O)C(O)CH₃ |
| AA | —CH(α-CN)— | —C(O)— | —OC(O)-n-propyl |
| AB | —CH(α-CN)— | —C(O)— | —NHC(O)C(O)CH₃ |
| AC | —C(O)— | —C(O)— | —OC(O)-n-propyl |
| AD | —C(O)— | —C(O)— | —NHC(O)C(O)CH₃ |
| AE | —CH(α-OH)— | —CH₂— | —OC(O)-n-propyl |
| AF | —CH(α-OH)— | —CH₂— | —NHC(O)C(O)CH₃ |
| AG | —CH(α-CN)— | —CH₂— | —OC(O)-n-propyl |
| AH | —CH(α-CN)— | —CH₂— | —NHC(O)C(O)CH₃ |
| AI | —C(O)— | —CH₂— | —OC(O)-n-propyl |
| AJ | —C(O)— | —CH₂— | —NHC(O)C(O)CH₃ |
| EN | —CH(α-OH)— | —C(O)— | —O-benzyl |
| EO | —CH(α-CN)— | —C(O)— | —O-benzyl |
| EP | —C(O)— | —C(O)— | —O-benzyl |
| EQ | —CH(α-OH)— | —CH₂— | —O-benzyl |
| ER | —CH(α-CN)— | —CH₂— | —O-benzyl |
| ES | —C(O)— | —CH₂— | —O-benzyl |

Additional illustrative compounds of formula (III) include the following:

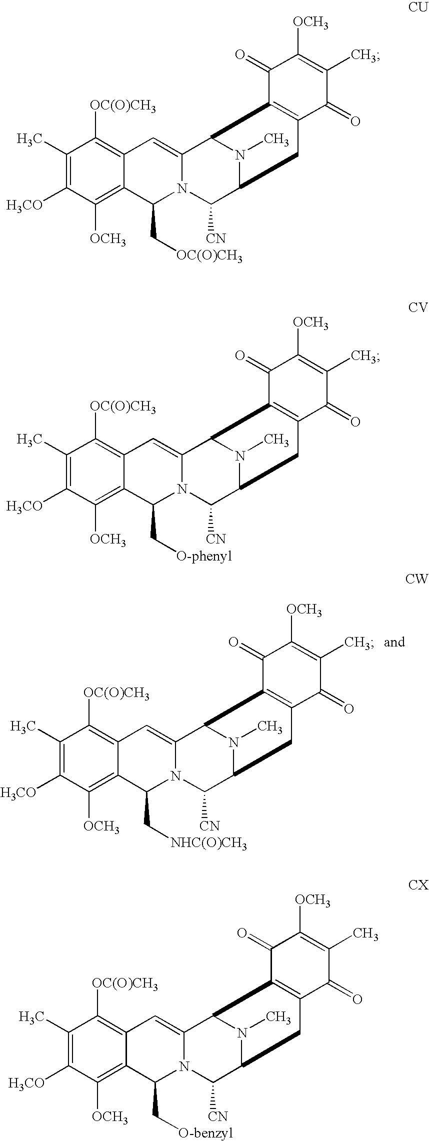

and pharmaceutically acceptable salts thereof.

### 4.18 Pentacyclic Alkaloid Compounds of Formula (IV)

As stated above, the present invention the present invention provides Pentacyclic Alkaloid Compounds of Formula (IV):

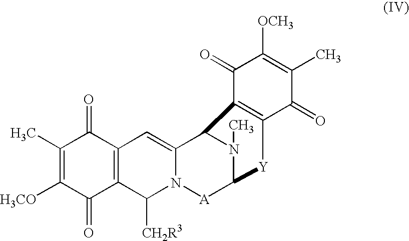

and pharmaceutically acceptable salts thereof,
where R³, A and Y are defined above for the Pentacyclic Alkaloid Compounds of Formula (IV).

In one embodiment R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl or

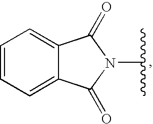

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In yet another embodiment R³ is —OC(O)—(C₁-C₁₂ alkyl).

In still another R³ is —NHC(O)—C₁-C₁₂ alkyl.

In a specific embodiment R³ is —NHC(O)-aryl.

In a further embodiment R³ is —NHC(O)-phenyl.

In one embodiment R³ is —C(O)—NH—C(O)—C₁-C₁₂ alkyl.

In still another embodiment R³ is —O-aryl.

In a specific embodiment R³ is —O-phenyl.

In yet another embodiment R³ is:

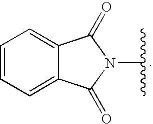

In one embodiment, the —CH₂R³ is in the α-configuration, i.e., below the plane, of the Pentacyclic Alkaloid Compound of formula (IV) as depicted.

In another embodiment, the —CH₂R³ is in the β-configuration, i.e., above the plane, of the Pentacyclic Alkaloid Compound of formula (IV) as depicted.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In yet another embodiment A and Y are each —C(O)—.

In one embodiment, A is —CH(α-OH)— and Y is —CH₂—.

In another embodiment, A is —CH(α-OH)— and Y is —C(O)—.

In one embodiment, the compound of formula (IV) has the formula (IVa):

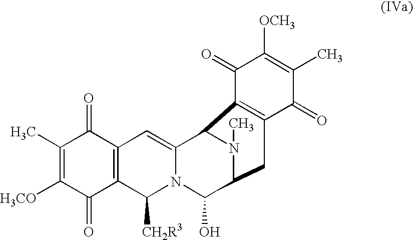

wherein R³ is defined above for the compounds of formula (IV).

In another embodiment, the compound of formula (IV) has the formula (IVb):

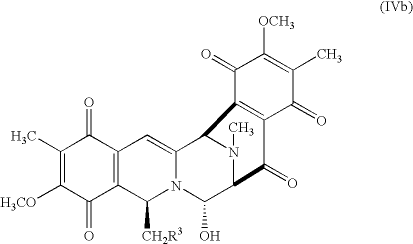

wherein R³ is defined above for the compounds of formula (IV).

Illustrative compounds of formula (IV) include the following:

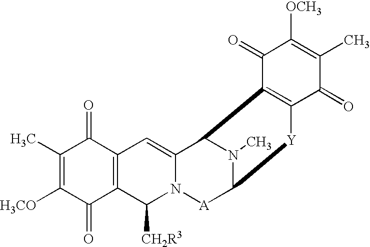

| Compound | A | Y | R³ |
|---|---|---|---|
| AK | —CH(α-OH)— | —C(O)— | —NHC(O)CH₃ |
| AL | —CH(α-OH)— | —C(O)— | —N-phthalimido |
| AM | —CH(α-CN)— | —C(O)— | —NHC(O)CH₃ |
| AN | —CH(α-CN)— | —C(O)— | —N-phthalimido |
| AO | —C(O)— | —C(O)— | —NHC(O)CH₃ |
| AP | —C(O)— | —C(O)— | —N-phthalimido |
| AQ | —CH(α-OH)— | —CH₂— | —NHC(O)CH₃ |
| AR | —CH(α-OH)— | —CH₂— | —N-phthalimido |
| AS | —CH(α-CN)— | —CH₂— | —NHC(O)CH₃ |
| AT | —CH(α-CN)— | —CH₂— | —N-phthalimido |
| AU | —C(O)— | —CH₂— | —NHC(O)CH₃ |
| AV | —C(O)— | —CH₂— | —N-phthalimido |
| ET | —CH(α-OH)— | —C(O)— | —O-benzyl |
| EU | —CH(α-CN)— | —C(O)— | —O-benzyl |
| EV | —C(O)— | —C(O)— | —O-benzyl |
| EW | —CH(α-OH)— | —CH₂— | —O-benzyl |
| EX | —CH(α-CN)— | —CH₂— | —O-benzyl |
| EY | —C(O)— | —CH₂— | —O-benzyl |

Additional illustrative compounds of formula (IV) include the following:

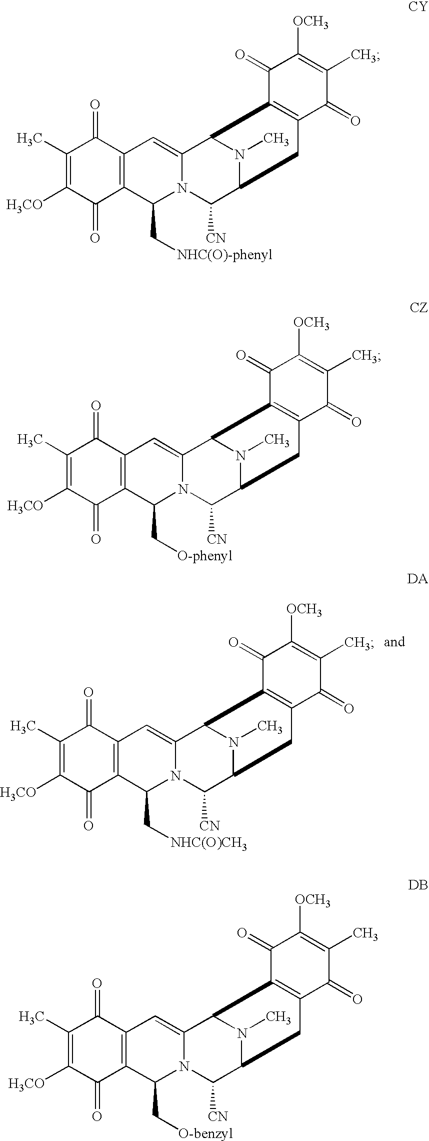

and pharmaceutically acceptable salts thereof.

### 4.19 Pentacyclic Alkaloid Compounds of Formula (V)

As stated above, the present invention the present invention provides Pentacyclic Alkaloid Compounds of Formula (V):

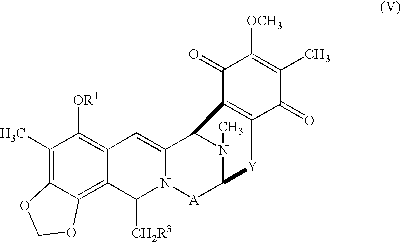

and pharmaceutically acceptable salts thereof,
where R¹, R³, A and Y are defined above for the Pentacyclic Alkaloid Compounds of Formula (V).

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R¹ is -allyl.

In one embodiment R¹ is —H.

In yet another embodiment R¹ is —C₁-C₁₂ alkyl.

In still another embodiment R¹ is —C(O)-aryl.

In yet another embodiment R¹ is —C(O)—C₁-C₁₂ alkyl.

In a specific embodiment R¹ is —C(O)—CH₃.

In a further embodiment R¹ is —SO₂CH₃.

In one embodiment R³ is —OC(O)—(C₁-C₁₂ alkyl), —NHC(O)—(C₁-C₁₂ alkyl), —NHC(O)—aryl, —NHC(O)—C(O)—(C₁-C₁₂ alkyl), —C(O)—HN—C(O)—(C₁-C₁₂ alkyl), —O-aryl or

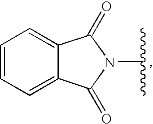

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In one embodiment R³ is —OC(O)—C₁-C₁₂ alkyl.

In a specific embodiment R³ is —OC(O)—CH₃.

In another embodiment R³ is —NHC(O)—C₁-C₁₂ alkyl.

In still another embodiment R³ is —NHC(O)-aryl.

In yet another embodiment R³ is —NHC(O)—C(O)—C₁-C₁₂ alkyl.

In a further embodiment R³ is —C(O)—NH—C(O)—C₁-C₁₂ alkyl.

In still another embodiment R³ is —O-aryl.

In a specific embodiment R³ is —O-phenyl.

In yet another embodiment R³ is:

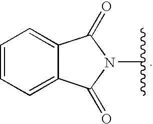

In one embodiment, the —CH₂R³ is in the α-configuration, i.e., below the plane of the Pentacyclic Alkaloid Compound of formula (V) as depicted.

In another embodiment, the —CH₂R³ is in the β-configuration, i.e., above the plane of the Pentacyclic Alkaloid Compound of formula (V) as depicted.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In yet another embodiment A and Y are each —C(O)—.

In another embodiment R¹ is —H.

In another embodiment R¹ is —H, and A is —C(O)—.

In still another embodiment R′ is —H, and A and Y are each —C(O)—.

In one embodiment, R¹ is —H.

In another embodiment, R¹ is —H; A is —CH(α-OH)—; and Y is —C(O)—.

In yet another embodiment, R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

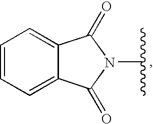

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In one embodiment, the compound of formula (V) has the formula (Va):

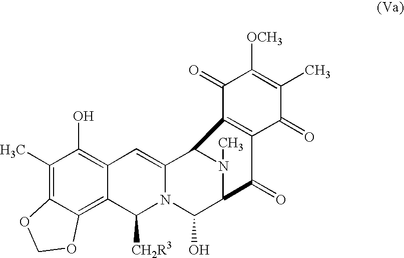

In another embodiment, the compound of formula (V) has the formula (Vb):

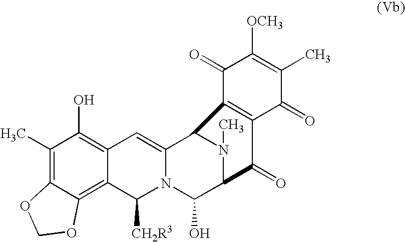

wherein R³ is defined above for the compounds of formula (V).

Illustrative compounds of formula (V) include the following:

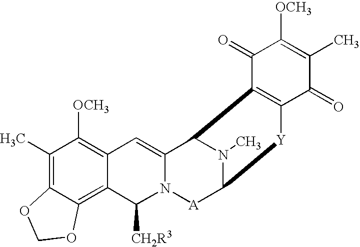

| Compound | A | Y | R³ |
|---|---|---|---|
| AW | —CH(α-OH)— | —C(O)— | —OC(O)-n-propyl |
| AX | —CH(α-OH)— | —C(O)— | —NHC(O)C(O)CH₃ |
| AY | —CH(α-CN)— | —C(O)— | —OC(O)-n-propyl |
| AZ | —CH(α-CN)— | —C(O)— | —NHC(O)C(O)CH₃ |
| BA | —C(O)— | —C(O)— | —OC(O)-n-propyl |
| BB | —C(O)— | —C(O)— | —NHC(O)C(O)CH₃ |
| BC | —CH(α-OH)— | —CH₂— | —OC(O)-n-propyl |
| BD | —CH(α-OH)— | —CH₂— | —NHC(O)C(O)CH₃ |
| BE | —CH(α-CN)— | —CH₂— | —OC(O)-n-propyl |
| BF | —CH(α-CN)— | —CH₂— | —NHC(O)C(O)CH₃ |
| BG | —C(O)— | —CH₂— | —OC(O)-n-propyl |
| BH | —C(O)— | —CH₂— | —NHC(O)C(O)CH₃ |
| EZ | —CH(α-OH)— | —C(O)— | —O-benzyl |
| FA | —CH(α-CN)— | —C(O)— | —O-benzyl |
| FB | —C(O)— | —C(O)— | —O-benzyl |
| FC | —CH(α-OH)— | —CH₂— | —O-benzyl |
| FD | —CH(α-CN)— | —CH₂— | —O-benzyl |
| FE | —C(O)— | —CH₂— | —O-benzyl |

Additional illustrative compounds of formula (V) include the following:

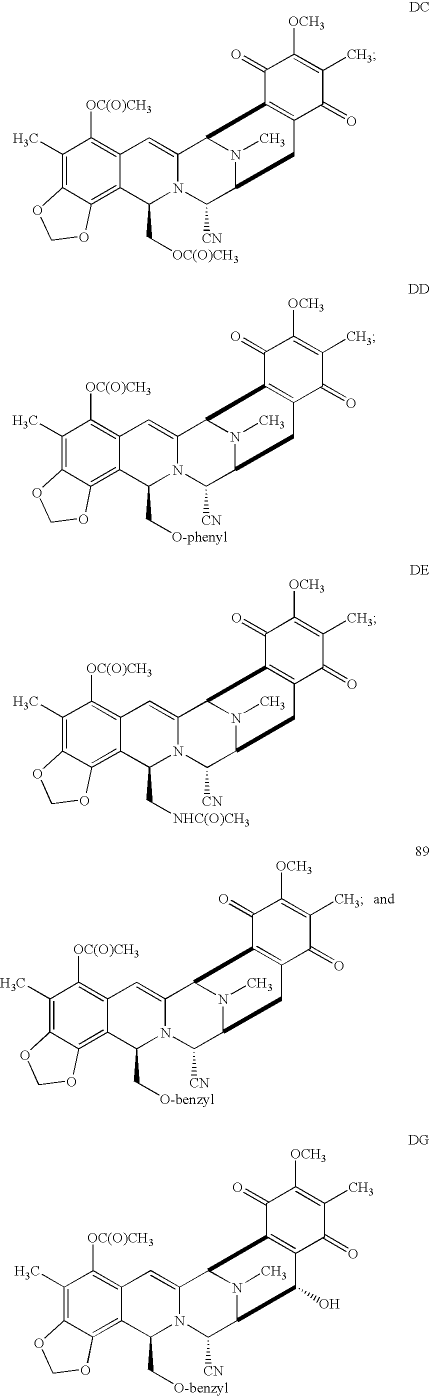

and pharmaceutically acceptable salts thereof.

### 4.20 Pentacyclic Alkaloid Compounds of Formula (VI)

As stated above, the present invention the present invention provides Pentacyclic Alkaloid Compounds of Formula (VI):

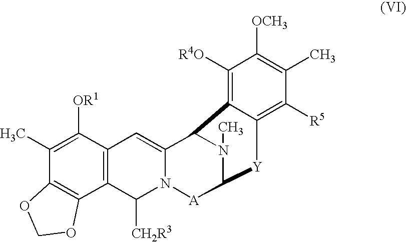

and pharmaceutically acceptable salts thereof,
where R¹, R³, R⁴, R⁵, A and Y are defined above for the Pentacyclic Alkaloid Compounds of Formula (VI).

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R¹ is allyl.

In a specific embodiment R¹ is —H.

In another embodiment R¹ is —C₁-C₁₂ alkyl.

In still another embodiment R¹ is —C(O)-aryl.

In yet another embodiment R¹ is —C(O)—C₁-C₁₂ alkyl.

In a specific embodiment R¹ is <(O)—CH₃.

In a further embodiment R¹ is —SO₂CH₃.

In one embodiment R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

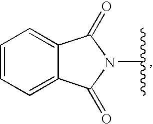

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In yet another embodiment R³ is —OC(O)—C₁-C₁₂ alkyl.

In a specific embodiment R³ is —OC(O)—CH₃.

In another embodiment R³ is —NHC(O)—C₁-C₁₂ alkyl.

In still another embodiment R³ is —NHC(O)-aryl.

In yet another embodiment R³ is —NHC(O)—C(O)—C₁-C₁₂ alkyl.

In a further embodiment R³ is —C(O)—NH—C(O)—C₁-C₁₂ alkyl.

In still another embodiment R³ is —O-aryl.

In a specific embodiment R³ is —O-phenyl.

In yet another embodiment R³ is:

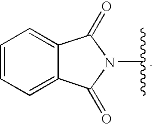

In one embodiment, the —CH₂R³ is in the α-configuration, i.e., below the plane, of the Pentacyclic Alkaloid Compound of formula (VI) as depicted.

In another embodiment, the —CH₂R³ is in the β-configuration, i.e., above the plane, of the Pentacyclic Alkaloid Compound of formula (VI) as depicted.

In one embodiment R⁴ is —H or —C₁-C₁₂ alkyl.

In another embodiment R⁴ is -benzyl.

In yet another embodiment R⁴ is —H.

In still another embodiment R⁴ is —C₁-C₁₂ alkyl.

In one embodiment R⁴ is —H.

In another embodiment R⁵ is —OH.

In still another embodiment R⁵ is —O—C₁-C₁₂ alkyl.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In another embodiment R¹ is —H.

In another embodiment R¹ is —H, and A is —C(O)—.

In still another embodiment R¹ is —H, and A and Y are each —C(O)—.

In one embodiment R⁴ is —H, and R⁵ is —OH.

In one embodiment, R¹ and R⁴ are each —H.

In another embodiment, R¹, R⁴ and R⁵ are each —H.

In still another embodiment, R¹ and R⁴ are each —H; A is CH(α-OH)—; and Y is —C(O)—.

In a further embodiment, R¹ and R⁴ are each —H; A is —CH(α-OH)—; and Y is —CH₂—.

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

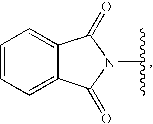

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R⁴ is —H or —C₁-C₁₂ alkyl.

In one embodiment, the compound of formula (VI) has the formula (VIa):

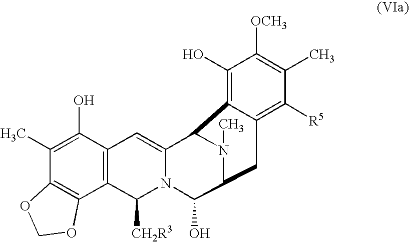

wherein R³ and R⁵ are defined above for the compounds of formula (VI).

In another embodiment, the compound of formula (VI) has the formula (VIb):

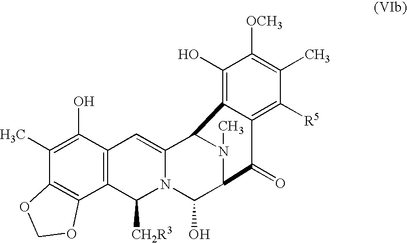

wherein R³ and R⁵ are defined above for the compounds of formula (VI).

Illustrative compounds of formula (VI) include the following:

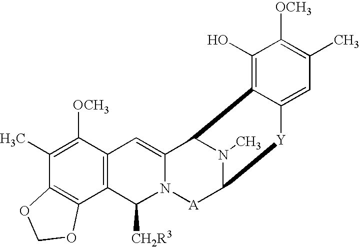

| Compound | A | Y | R³ |
|---|---|---|---|
| BI | —CH(α-OH)— | —C(O)— | —OC(O)-n-propyl |
| BJ | —CH(α-OH)— | —C(O)— | —NHC(O)C(O)CH₃ |
| BK | —CH(α-CN)— | —C(O)— | —OC(O)-n-propyl |
| BL | —CH(α-CN)— | —C(O)— | —NHC(O)C(O)CH₃ |
| BM | —C(O)— | —C(O)— | —OC(O)-n-propyl |
| BN | —C(O)— | —C(O)— | —NHC(O)C(O)CH₃ |
| BO | —CH(α-OH)— | —CH₂— | —OC(O)-n-propyl |
| BP | —CH(α-OH)— | —CH₂— | —NHC(O)C(O)CH₃ |
| BQ | —CH(α-CN)— | —CH₂— | —OC(O)-n-propyl |
| BR | —CH(α-CN)— | —CH₂— | —NHC(O)C(O)CH₃ |
| BS | —C(O)— | —CH₂— | —OC(O)-n-propyl |
| BT | —C(O)— | —CH₂— | —NHC(O)C(O)CH₃ |
| FF | —CH(α-OH)— | —C(O)— | —O-benzyl |
| FG | —CH(α-CN)— | —C(O)— | —O-benzyl |
| FH | —C(O)— | —C(O)— | —O-benzyl |
| FI | —CH(α-OH)— | —CH₂— | —O-benzyl |
| FJ | —CH(α-CN)— | —CH₂— | —O-benzyl |
| FK | —C(O)— | —CH₂— | —O-benzyl |

Additional illustrative compounds of formula (VI) include the following:

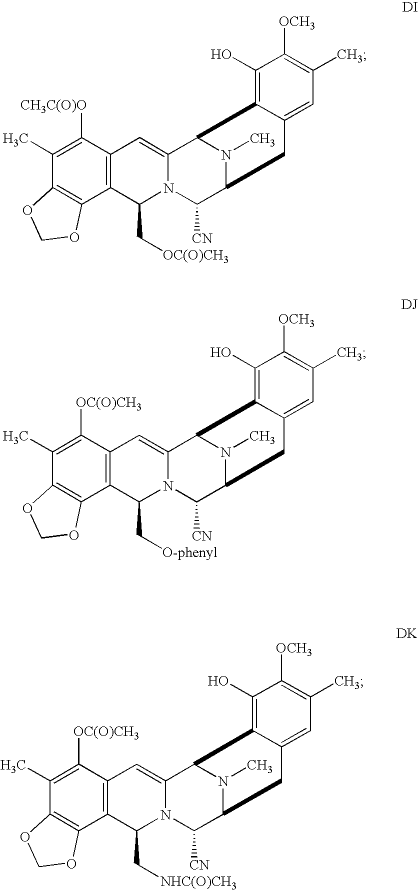

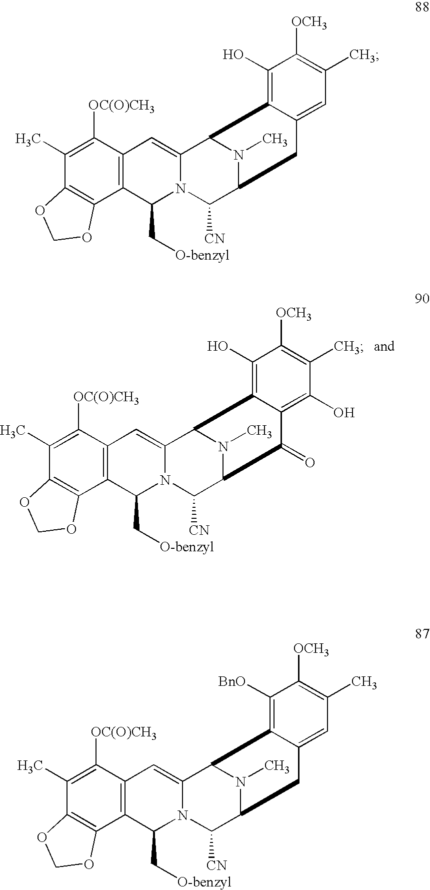

and pharmaceutically acceptable salts thereof.

### 4.21 Pentacyclic Alkaloid Compounds of Formula (VII)

As stated above, the present invention provides Pentacyclic Alkaloid Compounds of Formula (VII):

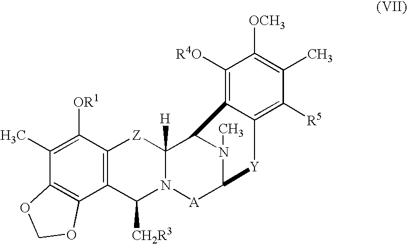

and pharmaceutically acceptable salts thereof,
where R¹, R³, R⁴, R⁵, A, Y, and Z are defined above for the Pentacyclic Alkaloid Compounds of Formula (VII).

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R¹ is allyl.

In a specific embodiment R¹ is —H.

In another embodiment R¹ is C₁-C₁₂ alkyl.

In still another embodiment R¹ is —C(O)-aryl.

In yet another embodiment R¹ is —C(O)—C₁-C₁₂ alkyl.

In a specific embodiment R¹ is —C(O)—CH₃.

In a further embodiment R¹ is —SO₂CH₃.

In one embodiment R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

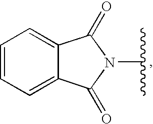

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl.

In another embodiment R³ is —O-benzyl.

In yet another embodiment R³ is —OC(O)—C₁-C₁₂ alkyl.

In a specific embodiment R³ is —OC(O)—CH₃.

In another embodiment R³ is —NHC(O)—C₁-C₁₂ alkyl.

In still another embodiment R³ is —NHC(O)-aryl.

In yet another embodiment R³ is —NHC(O)—C(O)—C₁-C₁₂ alkyl.

In a further embodiment R³ is -(O)—NH—C(O)—C₁-C₁₂ alkyl.

In still another embodiment R³ is —O-aryl.

In a specific embodiment R³ is —O-phenyl.

In yet another embodiment R³ is:

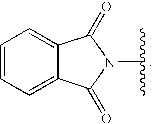

In one embodiment, the —CH₂R³ is in the α-configuration, i.e., below the plane, of the Pentacyclic Alkaloid Compound of formula (VII) as depicted.

In another embodiment, the —CH₂R³ is in the β-configuration, i.e., above the plane, of the Pentacyclic Alkaloid Compound of formula (VII) as depicted.

In one embodiment R⁴ is —H or —C₁-C₁₂ alkyl.

In another embodiment R⁴ is -benzyl.

In yet another embodiment R⁴ is —H.

In still another embodiment R⁴ is —C₁-C₁₂ alkyl.

In one embodiment R⁵ is —H.

In another embodiment R⁵ is —OH.

In still another embodiment R¹ is —O—C₁-C₁₂ alkyl.

In one embodiment, A is —CH₂—.

In another embodiment A is —CH(α-OH)—.

In still another embodiment A is —C(O)—.

In yet another embodiment A is —CH(α-CN)—.

In one embodiment, Y is —CH₂—.

In another embodiment Y is —CH(α-OH)—.

In still another embodiment Y is —C(O)—.

In one embodiment Z is —C(O)—.

In another embodiment Z is —CH(OH)—.

In yet another embodiment Z is —CH(α-OH)—.

In still another embodiment Z is —CH(β—OH)—.

In another embodiment R¹ is —H, and A is —C(O)—.

In still another embodiment R¹ is —H, and A and Y are each —C(O)—.

In one embodiment R⁴ is —H, and R⁵ is —OH.

In one embodiment, R¹ and R⁴ are each —H.

In another embodiment, R¹, R⁴ and R⁵ are each —H.

In still another embodiment, R¹ and R⁴ are each —H; A is —CH(α-OH)—; and Y is —C(O)—.

In a further embodiment, R¹ and R⁴ are each —H; A is —CH(α-OH)—; and Y is —CH₂—.

In one embodiment R¹ is —H, —C₁-C₁₂ alkyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl or

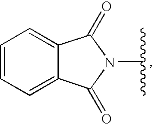

wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl; and R⁴ is —H or —C₁-C₁₂ alkyl; and R⁴ is —H or —C₁-C₁₂ alkyl.

In one embodiment, the compound of formula (VII) has the formula (VIIa):

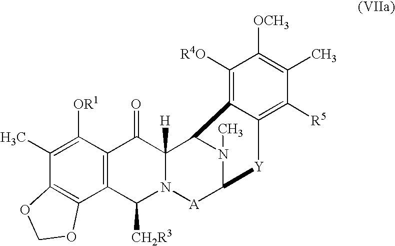

wherein R¹, R³, R⁴ and R⁵ are defined above for the compounds of formula (VII).

In one embodiment, the compound of formula (VII) has the formula (VIIb):

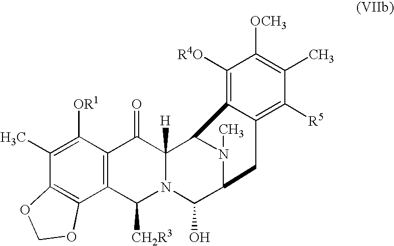

wherein R¹, R³, R⁴ and R⁵ are defined above for the compounds of formula (VII).

In another embodiment, the compound of formula (VII) has the formula (VIIc):

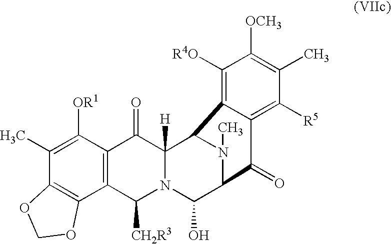

wherein R¹, R³, R⁴ and R⁵ are defined above for the compounds of formula (VII).

In yet another embodiment, the compound of formula (VII) has the formula (VIId):

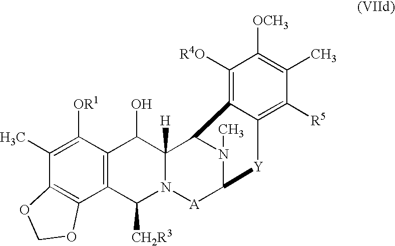

wherein R¹, R³, R⁴ and R¹ are defined above for the compounds of formula (VII).

In yet another embodiment, the compound of formula (VII) has the formula (VIIe):

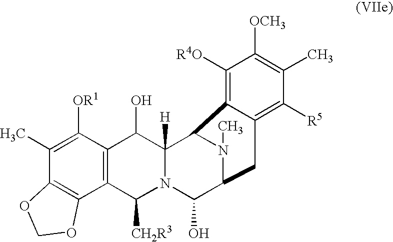

wherein R¹, R³, R⁴ and R⁵ are defined above for the compounds of formula (VII).

In still another embodiment, the compound of formula (VII) has the formula (VIIf):

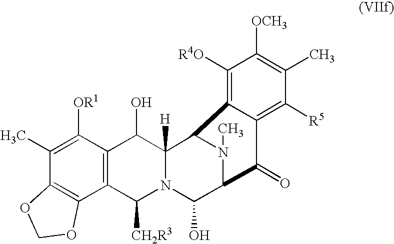

wherein R¹, R³, R⁴ and R⁵ are defined above for the compounds of formula (VII).

In a specific embodiment, the compound of formula (VII) has the formula (VIIg):

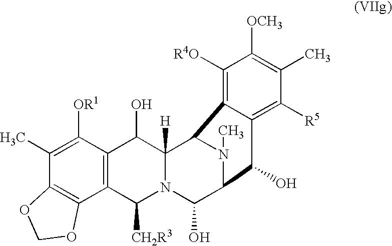

wherein R¹, R³, R⁴ and R⁵ are defined above for the compounds of formula (VII).

In yet another embodiment, the compound of formula (VII) has the formula (VIIh):

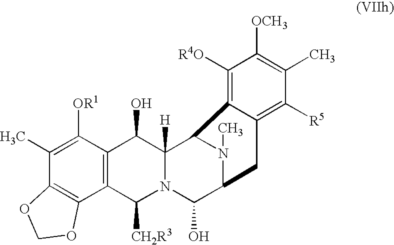

wherein R¹, R³, R⁴ and R⁵ are defined above for the compounds of formula (VII).

In still another embodiment, the compound of formula (VII) has the formula (VIIi):

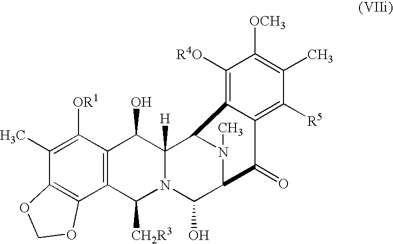

wherein R¹, R³, R⁴ and R⁵ are defined above for the compounds of formula (VII).

In still another embodiment, the compound of formula (VII) has the formula (VIIj):

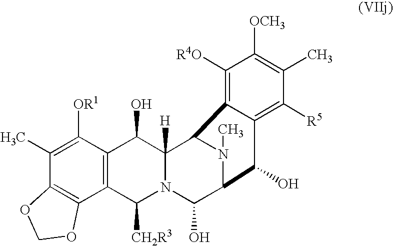

wherein R¹, R³, R⁴ and R⁵ are defined above for the compounds of formula (VII).

In still another embodiment, the compound of formula (VII) has the formula (VIIk):

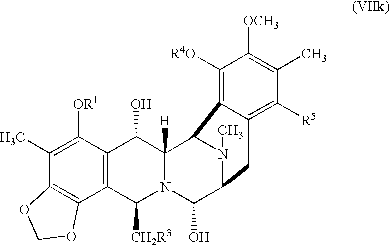

wherein R¹, R³, R⁴ and R⁵ are defined above for the compounds of formula (VII).

In still another embodiment, the compound of formula (VII) has the formula (VIIn):

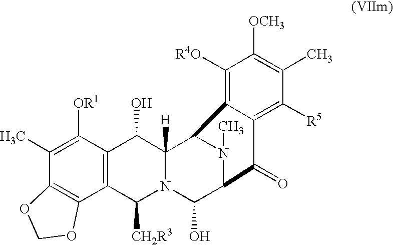

wherein R¹, R³, R⁴ and R⁵ are defined above for the compounds of formula (VII).

In still another embodiment, the compound of formula (VII) has the formula (VIIn):

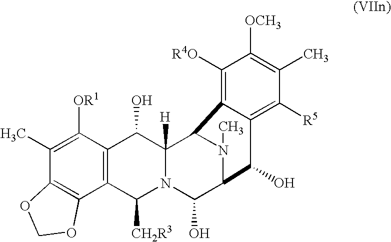

wherein R¹, R³, R⁴ and R⁵ are defined above for the compounds of formula (VII).

Illustrative compounds of formula (VII) include the following:

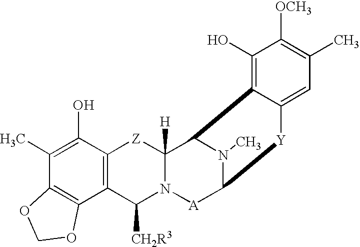

| Compound | Z | A | Y | R³ |
|---|---|---|---|---|
| BU | —C(O)— | —CH(α-OH)— | —C(O)— | —OC(O)-n-propyl |
| BY | —C(O)— | —CH(α-OH)— | —C(O)— | —NHC(O)C(O)CH₃ |
| BW | —C(O)— | —CH(α-CN)— | —C(O)— | —OC(O)-n-propyl |
| BX | —C(O)— | —CH(α-CN)— | —C(O)— | —NHC(O)C(O)CH₃ |
| BY | —C(O)— | —C(O)— | —C(O)— | —OC(O)-n-propyl |
| BZ | —C(O)— | —C(O)— | —C(O)— | —NHC(O)C(O)CH₃ |
| CA | —C(O)— | —CH(α-OH)— | —CH₂— | —OC(O)-n-propyl |
| CB | —C(O)— | —CH(α-OH)— | —CH₂— | —NHC(O)C(O)CH₃ |
| CD | —C(O)— | —CH(α-CN)— | —CH₂— | —OC(O)-n-propyl |
| CE | —C(O)— | —CH(α-CN)— | —CH₂— | —NHC(O)C(O)CH₃ |
| CF | —C(O)— | —C(O)— | —CH₂— | —OC(O)-n-propyl |
| CG | —C(O)— | —C(O)— | —CH₂— | —NHC(O)C(O)CH₃ |
| CH | —C(O)— | —CH(α-OH)— | —C(O)— | —O-benzyl |
| CI | —C(O)— | —CH(α-CN)— | —C(O)— | —O-benzyl |
| CJ | —C(O)— | —C(O)— | —C(O)— | —O-benzyl |
| CK | —C(O)— | —CH(α-OH)— | —CH₂— | —O-benzyl |
| CL | —C(O)— | —CH(α-CN)— | —CH₂— | —O-benzyl |
| CM | —C(O)— | —C(O)— | —CH₂— | —O-benzyl |
| FL | —CH(OH)— | —CH(α-OH)— | —C(O)— | —OC(O)-n-propyl |
| FM | —CH(OH)— | —CH(α-OH)— | —C(O)— | —NHC(O)C(O)CH₃ |
| FN | —CH(OH)— | —CH(α-CN)— | —C(O)— | —OC(O)-n-propyl |
| FO | —CH(OH)— | —CH(α-CN)— | —C(O)— | —NHC(O)C(O)CH₃ |
| FP | —CH(OH)— | —C(O)— | —C(O)— | —OC(O)-n-propyl |
| FQ | —CH(OH)— | —C(O)— | —C(O)— | —NHC(O)C(O)CH₃ |
| FR | —CH(OH)— | —CH(α-OH)— | —CH₂— | —OC(O)-n-propyl |
| FS | —CH(OH)— | —CH(α-OH)— | —CH₂— | —NHC(O)C(O)CH₃ |
| FT | —CH(OH)— | —CH(α-CN)— | —CH₂— | —OC(O)-n-propyl |
| FU | —CH(OH)— | —CH(α-CN)— | —CH₂— | —NHC(O)C(O)CH₃ |
| FV | —CH(OH)— | —C(O)— | —CH₂— | —OC(O)-n-propyl |
| FW | —CH(OH)— | —C(O)— | —CH₂— | —NHC(O)C(O)CH₃ |
| FX | —CH(OH)— | —CH(α-OH)— | —C(O)— | —O-benzyl |
| FY | —CH(OH)— | —CH(α-CN)— | —C(O)— | —O-benzyl |
| FZ | —CH(OH)— | —C(O)— | —C(O)— | —O-benzyl |
| GA | —CH(OH)— | —CH(α-OH)— | —CH₂— | —O-benzyl |
| GB | —CH(OH)— | —CH(α-CN)— | —CH₂— | —O-benzyl |
| GC | —CH(OH)— | —C(O)— | —CH₂— | —O-benzyl |

Additional illustrative compounds of formula (VII) include the following:

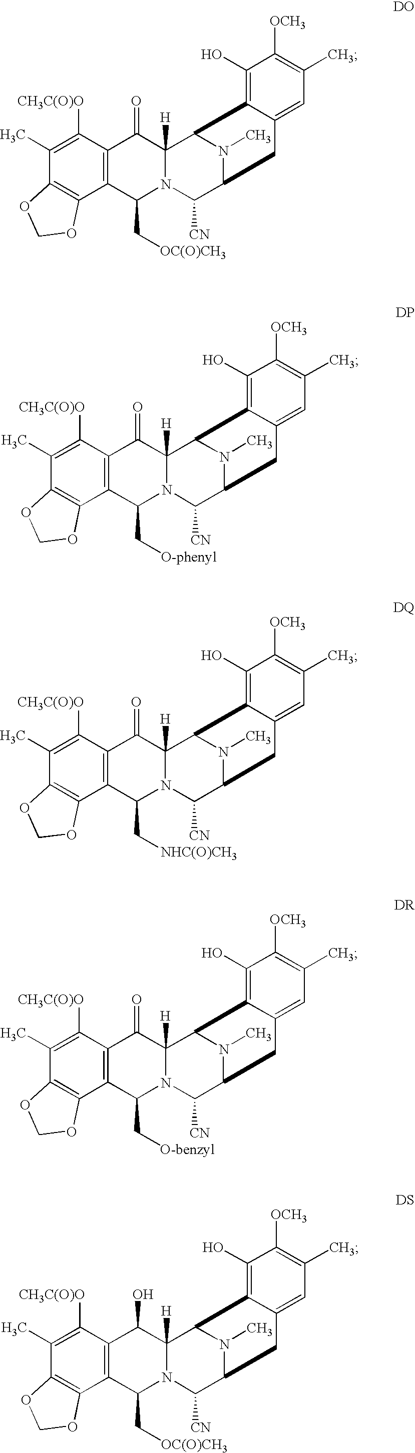

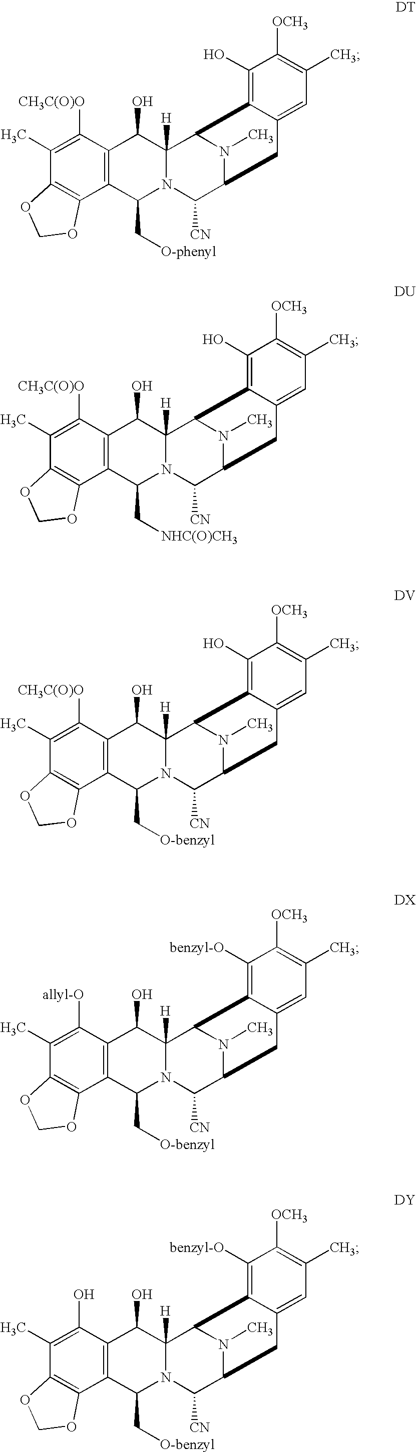

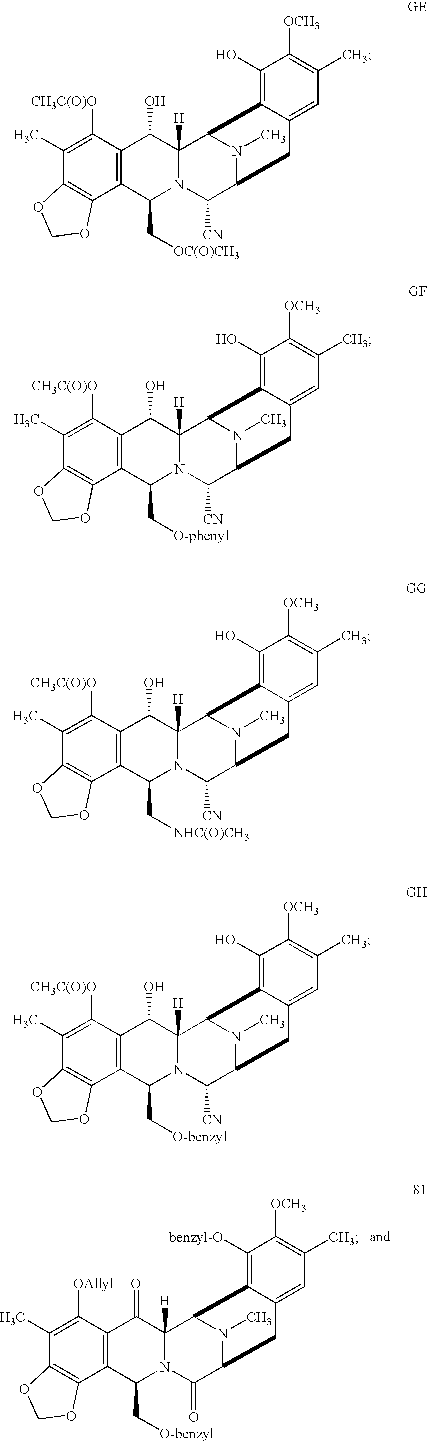

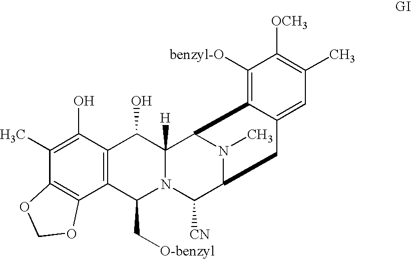

and pharmaceutically acceptable salts thereof.

### 4.22. Methods For Making The Pentacyclic Compounds of Formulas (I)-(VII)

### 4.22.1 Methods For Making The Compounds of Formulas (I)-(IV)

The compounds of formula (I), (II), (III) and (IV) can be made using the methods described in Schemes 1-4 above and using the methods of Schemes 5-15 below.

Scheme 5 shows methods for making the compounds of formula (I) or formula (III), wherein R¹ is —C₁-C₁₂ alkyl or -allyl, and R² is —CH₃.

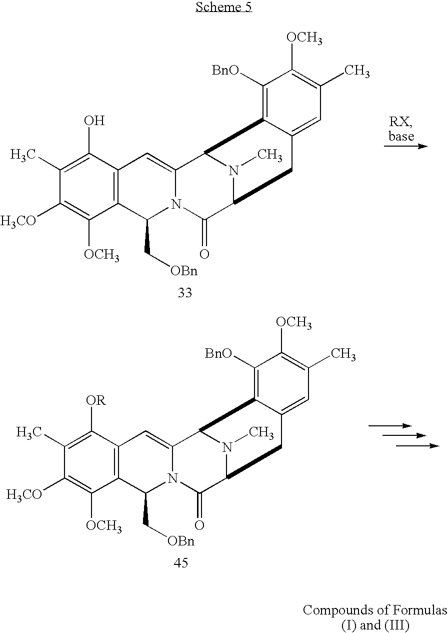

wherein R is —C₁-C₁₂ alkyl or -allyl; and X is a leaving group such as —Cl, —Br, —I, —O-mesyl, —O-tosyl, —O-brosyl, or —O-triflate.

A compound of formula 33 can be treated with a compound of formula RX in the presence of a base to provide a diether compound of formula 45. The compound of formula 45 can then be converted to a compound of formula (I) or formula (III), wherein R¹ is —C₁-C₁₂ alkyl or -allyl, and R² is —CH₃, using methods described above in Scheme 1.

Scheme 6 shows methods for making the compounds of formula (I) or formula (III), wherein R¹ and R² are each —C₁-C₁₂ alkyl

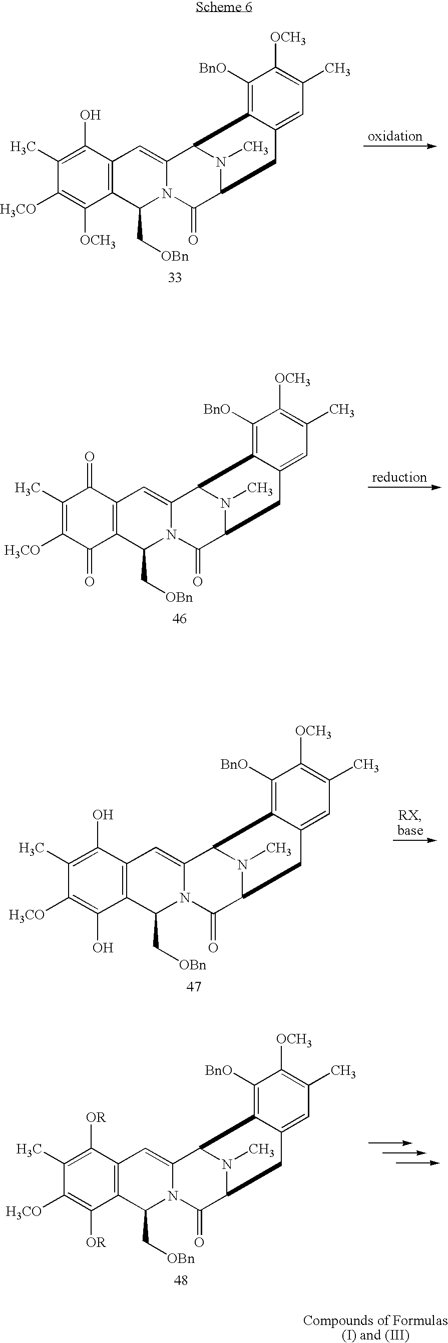

wherein R is —C₁-C₁₂ alkyl; and X is a leaving group such as —Cl, —Br, —I, —O-mesyl, —O-tosyl, —O-brosyl, or —O-triflate.

A compound of formula 33 can be oxidized, using, for example, Fremy's salt, to provide quinone compounds of formula 46, which can be subsequently reduced, using, for example, Zn/HOAc, to provide the corresponding hydroquinone compound of formula 47. The compound of formula 47 can then be reacted with a stoichiometric excess of a compound of formula RX in the presence of a base to provide the diether compounds of formula 48. The compounds of formula 48 can then be converted to compounds of formula (I) or formula (III), wherein R¹ and R² are each —C₁-C₁₂ alkyl, using methods described in Scheme 1.

Scheme 7 shows a method for making the compounds of formula (I) or formula (III), wherein R¹ and R² are each —C(O)—C₁-C₁₂ alkyl

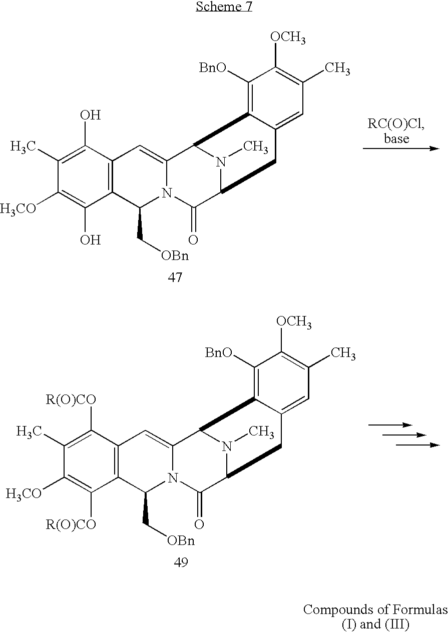

wherein R is —C₁-C₁₂ alkyl.

A compound of formula 47 is treated with a stoichiometric excess of an acid chloride of formula RC(O)Cl in the presence of a base to provide a diester compound of formula 49. The compounds of formula 49 can then be converted to compounds of formula (I) or formula (III), wherein R¹ and R² are each —C(O)—C₁-C₁₂ alkyl, using methods described in Scheme 1.

Scheme 8 shows a method for making the compounds of formula (I), wherein R¹ is —SO₂CH₃.

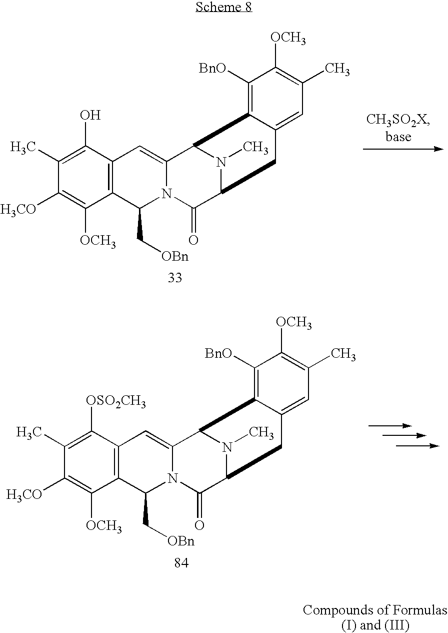

A compound of formula 33 is treated with a stoichiometric excess of a compound of general formula CH₃SO₂—X, wherein X is a leaving group such as —Cl, —Br, —I, —O-mesyl, —O-tosyl, —O-brosyl, or —O-triflate, in the presence of a base to provide a compound of formula 84. The compounds of formula 84 can then be converted to compounds of formula (I) or formula (III), wherein R¹ is —SO₂CH₃, using methods described in Scheme 1.

Compounds of formulas (I)-(IV), wherein R³ is —NHC(O)—C₁-C₁₂ alkyl, —NHC(O)-aryl, —NHC(O)—C(O)—C₁-C₁₂ alkyl, or -phthalimido can be made using the methodology depicted in Scheme 9.

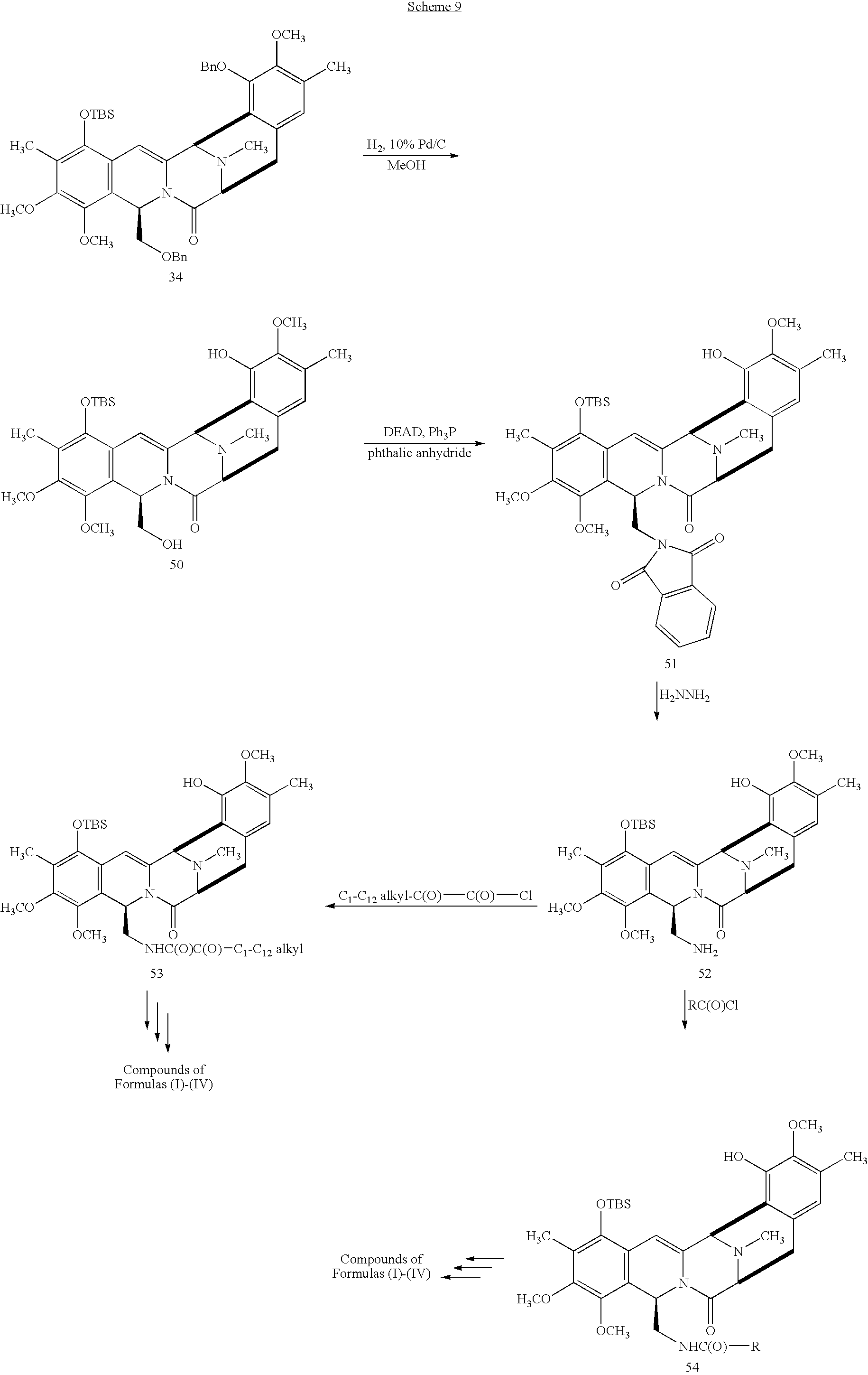

wherein R is —C₁-C₁₂ alkyl or aryl.

The dibenzyl compound of formula 34 can be debenzylated using 10% Pd/C to provide the dihydroxy compounds of formula 50. The hydroxymethyl group of the compound of formula 50 can be converted to a phthalimidomethyl group using a Mitsunobu process (see Simon et al., Tetrahedron 50:9757 (1994)) to provide the phthalimido compound of formula 51. The compound of formula 51 can then be reacted with hydrazine to provide the aminomethyl compound of formula 52. The compound of formula 52 can be reacted with: (1) an alkyl dicarbonyl chloride to provide an amine dione compound of formula 53; or (3) an alkyl or aryl acid chloride to provide an aryl or alkyl amido compound of formula 54. The compounds of formula 51, 53 or 54 can then be converted to compounds of formulas (I)-(IV), wherein R³ is —NHC(O)—C₁-C₁₂ alkyl, —NHC(O)-aryl, —NHC(O)—C(O)—C₁-C₁₂ alkyl, or -phthalimido, using methods described in Scheme 1.

Compounds of formulas (I)-(IV), wherein R³ is —O-aryl, can be made using the methodology shown in Scheme 10.

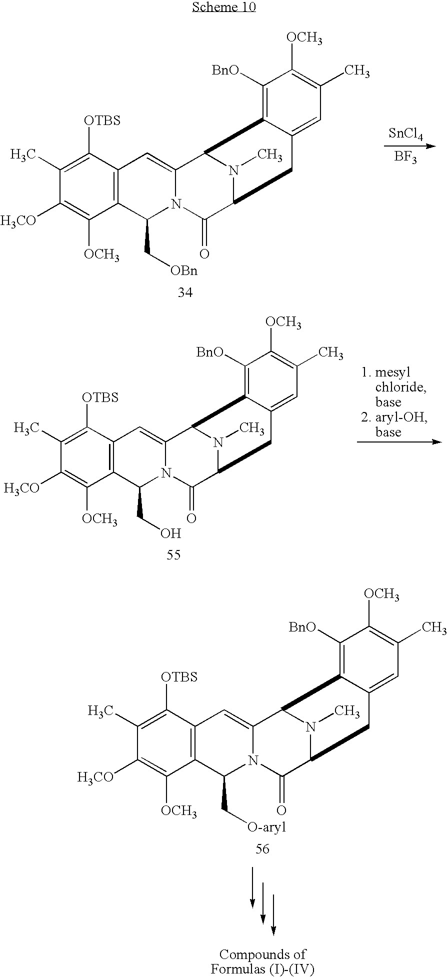

wherein X is a leaving group such as —Cl, —Br, —I, —O-mesyl, —O-tosyl, —O-brosyl, or —O-triflate.

The dibenzyl compound of formula 34 can be selectively deprotected using SnCl₄ in the presence of BF₃ to provide an the hydroxymethyl compound of formula 55. The compound of formula 55 can then be converted to its O-mesyl derivative using mesyl chloride, and the resultant mesylate can be reacted with a compound of formula aryl-OH in the presence of a base to provide a compound of formula 56. The compounds of formula 56 can then be converted to compounds of formulas (I)-(IV), wherein R³ is —O-aryl, using methods described in Scheme 1.

Compounds of formulas (I)-(III), wherein R³ is —O—C(O)—C₁-C₁₂ alkyl can be made using the methodology shown in Scheme 11.

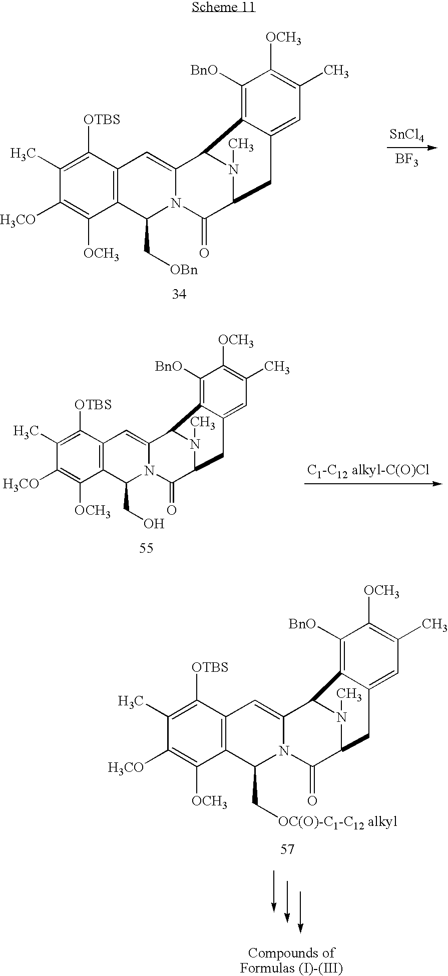

The dibenzyl compound of formula 34 can be selectively deprotected using SnCl₄ in the presence of BF₃ to provide the hydroxymethyl compound of formula 55. The compound of formula 55 can be reacted with a compound of formula —C₁-C₁₂ alkyl-C(O)Cl, in the presence of a base to provide an ester compound of formula 57. The compounds of formula 57 can then be converted to compounds of formulas (I)-(III), wherein R³ is —O—C(O)—C₁-C₁₂ alkyl, using methods described in Scheme 1.

Scheme 12 shows a method useful for making Compounds of formulas (I)-(IV), wherein R³ is —C(O)NHC(O)—C₁-C₁₂ alkyl.

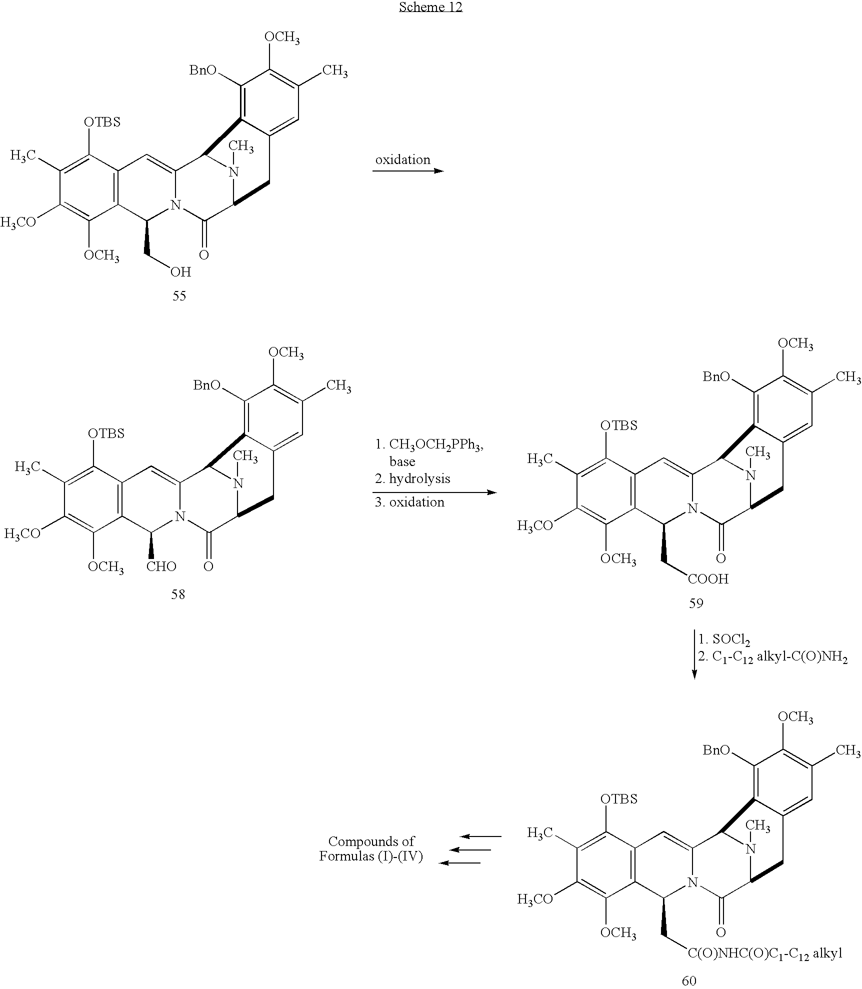

The hydroxymethyl compound of formula 55 can be oxidized using, for example, chromium trioxide, to provide an aldehyde of formula 58. The aldehyde of formula 58 can then be reacted with an ylide of formula CH₃OCH₂P⁺Ph₃ in a Wittig reaction and the resultant acetal hydrolyzed to provide a homologated aldehyde which is then oxidized using, for example, KMnO₄, to provide carboxylic acid 59. The carboxylic acid of formula 59 can be reacted with thionyl chloride and the resultant acyl chloride can then be coupled with an amide of formula C₁-C₁₂ alkyl-C(O)NH₂ to provide the imide of formula 60. The compounds of formula 60 can then be converted to compounds of formulas (I)-(IV), wherein R³ is —C(O)NHC(O)—C₁-C₁₂ alkyl, using methods described in Scheme 1.

Scheme 13 shows a method useful for making Compounds of formulas (I) or (II), wherein R⁴ is —C₁-C₁₂ alkyl and R⁵ is —H.

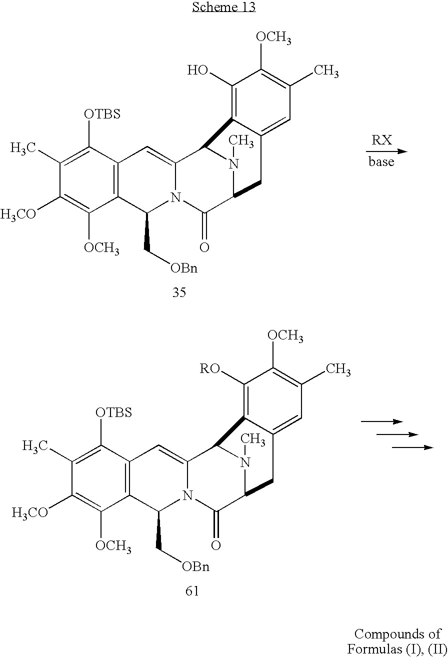

wherein R is —C₁-C₁₂ alkyl; and X is a leaving group such as —Cl, —Br, —I, —O-mesyl, —O-tosyl, —O-brosyl, or —O-triflate.

The compound of formula 35 can be treated with a compound of formula RX in the presence of a base to provide a diether compound of formula 61. A compound of formula 61 can then be converted to a compound of formula (I) or formula (II), wherein R⁴ is —C₁-C₁₂ alkyl and R⁵ is —H, using methods described above in Scheme 1.

Scheme 14 shows a method useful for making Compounds of formulas (I) and (II), wherein R⁵ is —OH or —O—C₁-C₁₂ alkyl.

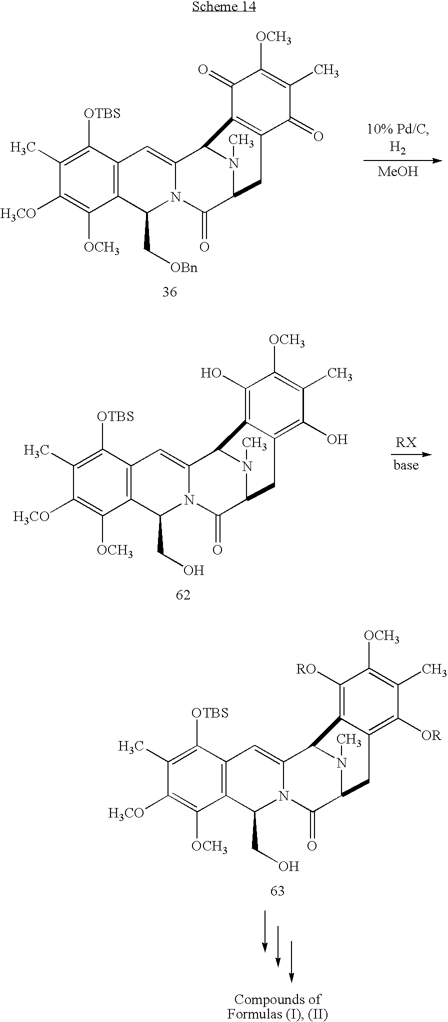

wherein R is —C₁-C₁₂ alkyl; and X is a leaving group such as —Cl, —Br, —I, —O-mesyl, —O-tosyl, —O-brosyl, or —O-triflate.

The quinone compound of formula 36 can be reduced to its corresponding hydroquinone compound of formula 59 using catalytic hydrogenation in the presence of 10% Pd/C. The compound of formula 62 can then be converted to a compound of formula (I) or formula (II), wherein R⁵ is —OH, using methods described above in Scheme 1. Alternatively, a compound of formula 62 can be treated with a stoichiometric excess of a compound of formula RX in the presence of a base to provide a diether compound of formula 63. A compound of formula 63 can then be converted to a compound of formula (I) or formula (II), wherein R⁵ is —O—C₁-C₁₂ alkyl, using methods described above in Scheme 1.

Scheme 15 shows methods useful for making the compounds of formulas (I) —(IV), wherein A is —CH₂—, CH(α-OH)— or —CH(α-CN)—.

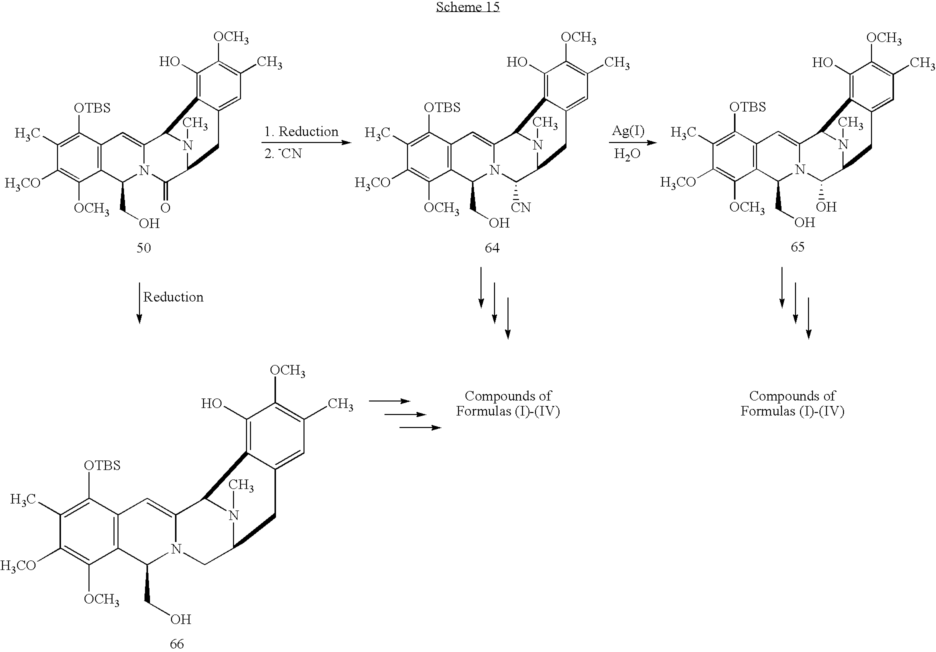

The lactam compound of formula 50 can be converted to its corresponding α-cyano analog of formula 64 via the following two step procedure: (1) partial reduction of the carbonyl using an aluminum reducing agent such as LiAlH₄, Red-Al, DIBAL-H, BuLi, or LiAlH(OtBu)₃, or a borane reducing agent, such as BH₃; and (2) reaction of the resultant hemiaminal with —CN. Sources of —CN include NaCN, KCN, and TMSCN/lewis acid. A cyano compound of formula 64 can be further converted to its corresponding α-hydroxy analog of formula 65 upon reaction with Ag(I) in the presence of water. Alternatively, the carbonyl group of the compound of formula 50 can be fully reduced to its corresponding methylene group via a Wolff-Kishner reduction, Clemmensen reduction, or by converting the carbonyl to its dithiane derivative and reacting the dithiane with Raney nickel. This provides a compound of formula 66. The compounds of formulas 64-66 can then be converted to compounds of formulas (I)-(IV), wherein A is —CH₂—, CH(α-OH)— or —CH(α-CN)—, using methods described in Scheme 1.

Scheme 16 shows methods useful for making the compounds of formulas (I)-(IV), wherein —CH₂R³ is in the α-configuration.

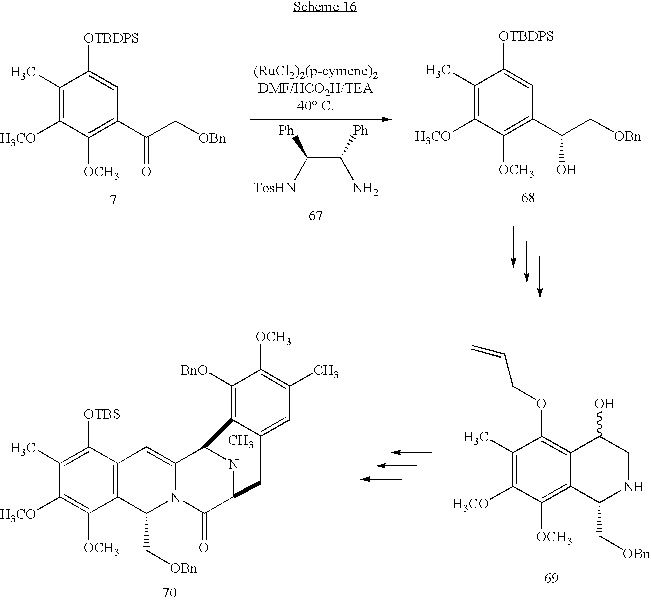

The carbonyl group of Compound 7 can be selective reduced using the indicated ruthenium catalyst in the presence of the compound of formula 67 to provide the α-hydroxy benzyl derivative of formula 68. Compound 68 can then be converted to bicyclic intermediate 69 (which is an isomer of Compound 14) using the methods described in Scheme 3. Following the method of Scheme 2 and substituting Compound 69 for Compound 14 provides the tetracyclic Compound 70 (which is the diastereomer of Compound 34). Compound 70 can be modified to obtain the compounds of formulas (I)-(IV), wherein —CH₂R³ is in the α-configuration, using methods described herein.

### 4.22.2 Methods For Making The Compounds of Formulas (V) and (VI)

The compounds of formulas (V) and (VI) can be made as depicted in Scheme 17.

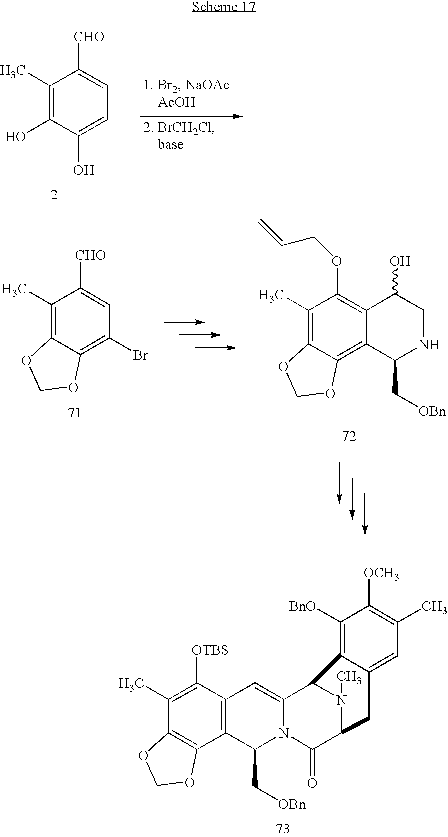

Diol Compound 2 can be brominated, then reacted with bromochloromethane to provide the methylene acetal Compound 71. Compound 71 can then be converted to bicyclic intermediate 72 (which is the methylene acetal analog of Compound 14) using the methods described in Scheme 3. Following the method of Scheme 2 and substituting Compound 72 for Compound 14 provides the tetracyclic Compound 73 (which is the methylene acetal analog of Compound 34). Compound 73 can be modified to obtain the compounds of formulas (V) and (VI), wherein —CH₂R³ is in the β-configuration, using methods described herein.

Scheme 18 shows methods useful for making the compounds of formulas (V) and (VI), wherein —CH₂R³ is in the α-configuration.

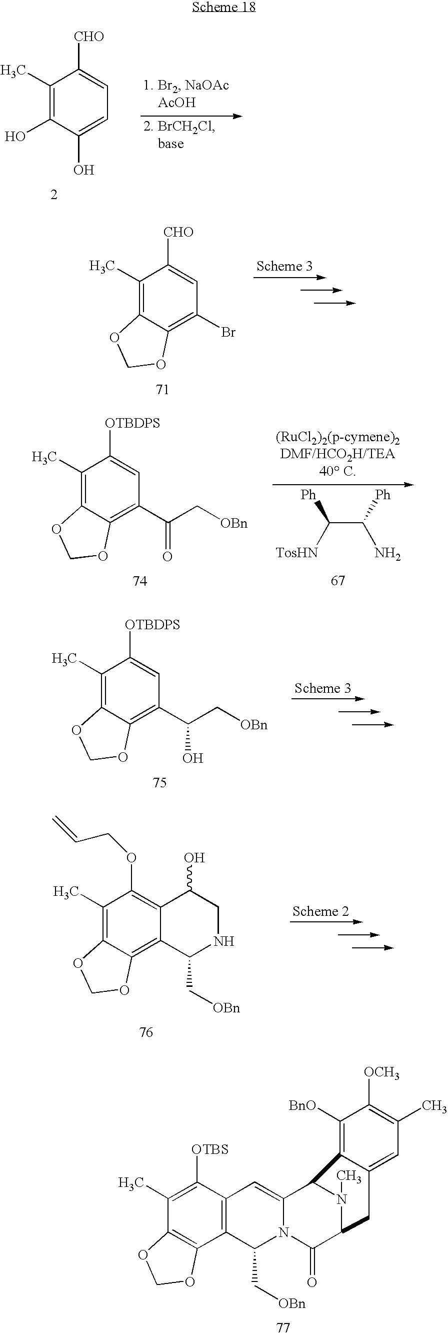

Compound 2 can be brominated, then reacted with bromochloromethane to provide the methylene acetal Compound 71. Compound 71 can then be carried forth to ketone Compound 74 (which is the methylene acetal analog of Compound 7) using the methods described in Scheme 3. The carbonyl group of Compound 74 can be selectively reduced using the indicated ruthenium catalyst in the presence of the compound of formula 67 to provide the α-hydroxy benzyl derivative of formula 75. Compound 75 can then be converted to bicyclic intermediate 76 (which is the analogous to Compound 14) using the methods described in Scheme 3. Following the method of Scheme 2 and substituting Compound 76 for Compound 14 provides the tetracyclic Compound 77 (which is analogous to Compound 34). Compound 77 can be modified to obtain the compounds of formulas (V) and (VI), wherein —CH₂R³ is in the α-configuration, using methods described herein.

Alternatively, the compounds of formulas (V) and (VI) wherein —CH₂R³ is in the α-configuration can be made through epimerization of Compound 73, using methods known to one of skill in the art, for example, by debenzylating the primary benzyloxy group, oxidizing the resultant primary alcohol to an aldehyde group, epimerizing the aldehyde group to its α-isomer with base, and reducing the α-aldehyde group to an alcohol. To the extent protection and deprotection are desirable to make compounds of formulas (V) and (VI), wherein —CH₂R³ is in the α-configuration, methods of protection and deprotection are known to one of skill in the art, some of which are described in T. W. Greene et al., Protective Groups in Organic Synthesis 17-200 (3d ed. 1999).

### 4.22.3 Methods For Making The Compounds of Formula (VII)

The compounds of formula (VII) can be made as depicted in Scheme 20.

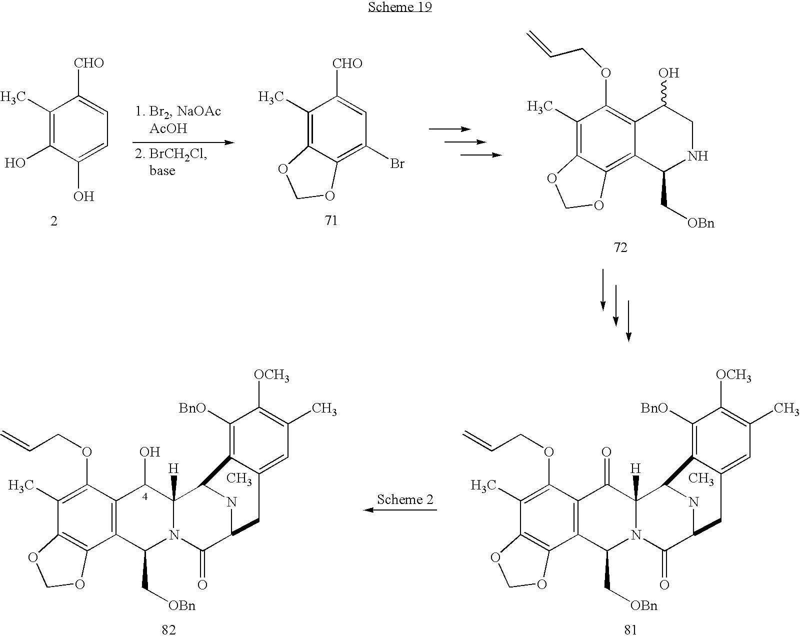

Compound 2 can be brominated, then reacted with bromochloromethane to provide Compound 71. Compound 71 can then be converted to Compound 72 using the methods for converting Compound 3 to Compound 14 described in Scheme 3. Compound 72 can be converted to Compound 81 following the methods for converting Compound 14 to Compound 31 described in Scheme 2. Compound 81 can be modified to obtain the compounds of formula (VII), wherein Z is —C(O)— and —CH₂R³ is in the β-configuration, using methods described herein.

Reduction of Compound 81, following methods shown in Scheme 2, yields the alcohol 82. The alcohol at carbon-4 of a compound of formula 82 (see Scheme 20, above) can be protected and deprotected, if necessary, using methods described in T. W. Greene et al., Protective Groups in Organic Synthesis 17-200 (3d ed. 1999). A protected compound of formula 82 can be modified and deprotected, if necessary, to obtain the compounds of formula (VII), wherein Z is —CH(OH)— and —CH₂R³ is in the β-configuration, using methods described herein.

The compounds wherein —CH₂R³ is in the α-configuration can also be obtained using methods described herein.

### 4.23 Therapeutic Uses of the Pentacyclic Alkaloid Compounds

### 4.23.1 Treatment or Prevention of Cancer

The Pentacyclic Alkaloid Compounds can be used to treat or prevent cancer. Examples of cancers treatable or preventable using the Pentacyclic Alkaloid Compounds include, but are not limited to, the cancers disclosed below in Table 1 and metastases thereof.

TABLE 1

Solid tumors, including but not limited to:
soft tissue sarcoma
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
gastric cancer
liver cancer
renal cancer
pharynx cancer
lung carcinoma
osteosarcoma
non-small cell lung cancer
melanoma
neuroblastoma
retinoblastoma
colon adenocarcinoma
malignant melanoma
blood-borne cancers, lymphomas, or leukemias,
including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
histiocytic lymphoma
Hodgkin's disease
non-Hodgkin's Lymphoma
Multiple myeloma
Waldenström's macroglobulinemia
Heavy chain disease
Polycythemia vera
Burkitts B cell lymphoma
undifferentiated lymphoma
T-cell lymphoma
hairy B cell lymphoma
acute and chronic leukemias:
lymphoblastic
myelogenous
lymphocytic
myelocytic leukemias

In one embodiment, the cancer is lung cancer, breast cancer, colon cancer, colorectal cancer, prostate cancer, pancreatic cancer, stomach cancer, kidney cancer, lymphoma, Hodgkin's disease, leukemia, testicular cancer, bladder cancer, head and neck cancer, soft tissue sarcoma or ovarian cancer.

In still another embodiment, the subject in need of treatment has previously undergone treatment for cancer. Such previous treatments include, but are not limited to, prior chemotherapy, radiation therapy, surgery or immunotherapy, such as cancer vaccines.

The Pentacyclic Alkaloid Compounds are also useful for the treatment or prevention of a cancer caused by a virus. For example, human papilloma virus can lead to cervical cancer (see, e.g., Hernandez-Avila et al., Archives of Medical Research (1997) 28:265-271), Epstein-Barr virus (EBV) can lead to lymphoma (see, e.g., Herrmann et al., J Pathol (2003) 199(2): 140-5), hepatitis B or C virus can lead to liver carcinoma (see, e.g., El-Serag, J Clin Gastroenterol (2002) 35(5 Suppl 2):S72-8), human T cell leukemia virus (HTLV)-I can lead to T-cell leukemia (see e.g., Mortreux et al., Leukemia (2003) 17(1):26-38), human herpesvirus-8 infection can lead to Kaposi's sarcoma (see, e.g., Kadow et al., Curr Opin Investig Drugs (2002) 3(11):1574-9), and Human Immune deficiency Virus (HUV) infection contribute to cancer development as a consequence of immunodeficiency (see, e.g., Dal Maso et al., Lancet Oncol (2003) 4(2):110-9).

### 4.23.2 Prophylactic Methods

The Pentacyclic Alkaloid Compounds can also be administered to prevent the progression of a cancer, including but not limited to the cancers listed in Table 1. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79).

In other embodiments, a subject which exhibits one or more of the following predisposing factors for malignancy can be treated by administration of an amount of a Pentacyclic Alkaloid Compound: a chromosomal translocation associated with a malignancy (e.g., the Philadelphia chromosome for chronic myelogenous leukemia, t(14; 18) for follicular lymphoma), familial polyposis or Gardner's syndrome, benign monoclonal, a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113), and exposure to carcinogens (e.g., smoking, and inhalation of or contacting with certain chemicals).

### 4.23.3 Combination Chemotherapy

The present methods for treating cancer can further comprise the administration of an effective amount of another anticancer agent.

The Pentacyclic Alkaloid Compound and the other anticancer agent can act additively or synergistically. A synergistic use of a Pentacyclic Alkaloid Compound and another anticancer agent permits the use of lower dosages of one or more of these agents and/or less frequent administration of the agents to a subject with cancer. The ability to utilize lower dosages of a Pentacyclic Alkaloid Compound and/or another anticancer agent and/or to administer the agents less frequently can reduce the toxicity associated with the administration of the agents to a subject without reducing the efficacy of the agents in the treatment of cancer. In addition, a synergistic effect can result in the improved efficacy of these agents in the treatment of cancer and/or the reduction of adverse or unwanted side effects associated with the use of either agent alone.

In one embodiment, the Pentacyclic Alkaloid Compound and the anticancer agent can act synergistically when administered in doses typically employed when such agents are used as monotherapy for the treatment of cancer. In another embodiment, the Pentacyclic Alkaloid Compound and the anticancer agent can act synergistically when administered in doses that are less than doses typically employed when such agents are used as monotherapy for the treatment of cancer.

Suitable additional anticancer agents useful in the methods and compositions of the present invention include, but are not limited to, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethyhmelamine, topotecan, tyrosine kinase inhibitors, tyrphostins herbimycin A, genistein, erbstatin, and lavendustin A.

In one embodiment, the additional anticancer agent can be, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| Platinum containing complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-β) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ATPase inhibitors: | Thapsigargin |

Other additional anticancer agents that can be used in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine hydrochloride; hydroxyurea; idarubicin hydrochloride; ifosfamide; ilmofosine; interleukin II (including recombinant interleukin II, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Further anticancer drugs that can be used in the methods and compositions of the invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta-lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetanide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

### 4.23.4 Multi-Modality Therapy For Cancer

The Pentacyclic Alkaloid Compounds can be administered to a subject that has undergone or is currently undergoing one or more additional anticancer treatment modalities including, but not limited to, surgery, radiation therapy, or immunotherapy, such as cancer vaccines.

In one embodiment, the invention provides methods for treating cancer comprising (a) administering to a subject in need thereof an amount of a Pentacyclic Alkaloid Compound effective to treat cancer; and (b) administering to said subject one or more additional anticancer treatment modalities including, but not limited to, surgery, radiation therapy, or immunotherapy, such as a cancer vaccine.

In one embodiment, the additional anticancer treatment modality is radiation therapy.

In another embodiment, the additional anticancer treatment modality is surgery.

In still another embodiment, the additional anticancer treatment modality is immunotherapy.

In a specific embodiment, the Pentacyclic Alkaloid Compounds are administered concurrently with radiation therapy. In another specific embodiment, the additional anticancer treatment modality is administered prior or subsequent to the Pentacyclic Alkaloid Compound, preferably at least an hour, five hours, 12 hours, a day, a week, a month, more preferably several months (e.g., up to three months), prior or subsequent to administration of the Pentacyclic Alkaloid Compounds.

When the additional anticancer treatment modality is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, the invention provides methods of treatment of cancer using the Pentacyclic Alkaloid Compounds as an alternative to chemotherapy or radiation therapy where the chemotherapy or the radiation therapy results in negative side effects, in the subject being treated. The subject being treated can, optionally, be treated with another anticancer treatment modality such as surgery, radiation therapy, or immunotherapy, depending on which treatment is found to be acceptable or bearable.

The Pentacyclic Alkaloid Compounds can also be used in an in vitro or ex vivo fashion, such as for the treatment of certain cancers, including, but not limited to leukemias and lymphomas, such treatment involving autologous stem cell transplants. This can involve a multi-step process in which the subject's autologous hematopoietic stem cells are harvested and purged of all cancer cells, the patient's remaining bone-marrow cell population is then eradicated via the administration of the Pentacyclic Alkaloid Compounds and/or high dose radiation therapy, and the stem cell graft is infused back into the subject. Supportive care can be provided while bone marrow function is restored and the subject recovers.

A Pentacyclic Alkaloid Compound and the other therapeutic agent can act additively or, in one embodiment synergistically. In one embodiment a Pentacyclic Alkaloid Compound is administered concurrently with another anticancer agent. In one embodiment a composition comprising an effective amount of a Pentacyclic Alkaloid Compound and an effective amount of another anticancer agent can be administered. Alternatively, a composition comprising an effective amount of a Pentacyclic Alkaloid Compound and a different composition comprising an effective amount of another anticancer agent can be concurrently administered. In another embodiment an, effective amount of a Pentacyclic Alkaloid Compound is administered prior or subsequent to administration of an effective amount of another anticancer agent. In this embodiment the Pentacyclic Alkaloid Compound is administered while the other therapeutic agent exerts its therapeutic effect, or the other therapeutic agent is administered while the Pentacyclic Alkaloid Compound exerts its preventative or therapeutic effect for treating or preventing cancer.

A composition of the invention can be prepared by a method comprising admixing a Pentacyclic Alkaloid Compound and a physiologically acceptable carrier or vehicle. Admixing can be accomplished using methods well known for admixing a compound (or salt) and a physiologically acceptable carrier or vehicle. In one embodiment the Pentacyclic Alkaloid Compound is present in the composition in an effective amount.

### 4.23.5 Treatment or Prevention of a Bacterial Infection

The Pentacyclic Alkaloid Compounds are useful for treating or preventing a bacterial infection. Illustrative types of bacteria involved in the infection include, but are not limited to, those disclosed below in Table 3.

TABLE 3

Bacillus subtilis
Streptococcus pneumoniae
Penicillin-resistant Streptococcus pneumoniae
Neisseria gonorrheae
Penicillin-resistant Neisseria gonorrheae
Group A Streptococcus
Staphylococcus aureus FDA 209P
Staphylococcus aureus Smith
Staphylococcus albus
Staphylococcus citreus
Streptococcus faecalis
Streptococcus pyogenes COOK
Streptococcus pyogenes 090R
Streptococcus salivarius
Sarcina lutea
Bacillus subtilis PCI 219
Bacillus cereus
Corynebacterium diptheriae
Corynebacterium xerosis
Mycobacterium sp. 607
Mycobacterium phlei
Mycobacterium avium
Nocardia asteroides
Escherichia coli F₁
Salmonella typhimurium
Shigella dysenteriae Shiga
Klebsiella pneumoniae
Brucella abortus
Serraria marcescens

### 4.23.5 Treatment or Prevention of a Fungal Infection

The Pentacyclic Alkaloid Compounds are useful for treating or preventing a fungal infection. Illustrative types of fungi involved in the infection include, but are not limited to, those disclosed below in Table 4.

TABLE 4

Rhodotorula glutinis
Penicillium glaucum
Trichophyton mentagrophytes

### 4.23.5 Treatment or Prevention of a Yeast Infection

The Pentacyclic Alkaloid Compounds are useful for treating or preventing a yeast infection. Illustrative types of yeast involved in the infection include, but are not limited to, those disclosed below in Table 5.

TABLE 5

Candida albicans 7N
Saccharomyces cerevisiae
Saccharomyces cerevisiae CCY333

### 4.24 Therapeutic/Prophylactic Administration and Compositions of the Invention

Due to their activity, the Pentacyclic Alkaloid Compounds are advantageously useful in veterinary and human medicine. As described above, the Pentacyclic Alkaloid Compounds are useful for treating or preventing a Condition in a subject in need thereof.

When administered to a subject, the Pentacyclic Alkaloid Compounds can be administered as a component of a composition that comprises a physiologically acceptable carrier or vehicle. The present compositions, which comprise a Pentacyclic Alkaloid Compound, can be administered orally. The Pentacyclic Alkaloid Compounds can also be administered by any other convenient route, for example, by infusion or bolus injection, by absorption through epithelial or mucocutaneous linings (e.g., oral, rectal, and intestinal mucosa) and can be administered together with another biologically active agent. Administration can be systemic or local. Various delivery systems are known, e.g., encapsulation in liposomes, microparticles, microcapsules, capsules, and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Pentacyclic Alkaloid Compounds into the bloodstream. The mode of administration can be left to the discretion of the practitioner.

In one embodiment, the Pentacyclic Alkaloid Compounds are administered orally.

In other embodiments, it can be desirable to administer the Pentacyclic Alkaloid Compounds locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Pentacyclic Alkaloid Compounds into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, the Pentacyclic Alkaloid Compounds can be formulated as a suppository, with traditional binders and excipients such as triglycerides.

In another embodiment the Pentacyclic Alkaloid Compounds can be delivered in a vesicle, in particular a liposome (see Langer, Science 249:1527-1533 (1990) and Liposomes in the Therapy of Infectious Disease and Cancer 317-327 and 353-365 (1989)).

In yet another embodiment the Pentacyclic Alkaloid Compounds can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Pentacyclic Alkaloid Compounds, e.g., the spinal column, brain, skin, lung, or gastrointestinal tract, thus requiring only a fraction of the systemic dose.

The present compositions can optionally comprise a suitable amount of a pharmaceutically acceptable excipient so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to a subject. Water is a particularly useful excipient when the Pentacyclic Alkaloid Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment the Pentacyclic Alkaloid Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving a Pentacyclic Alkaloid Compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Pentacyclic Alkaloid Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the Pentacyclic Alkaloid Compounds are to be administered by infusion, they can be dispensed, for example, with an infusion bottle containing sterile pharmaceutical grade water or saline. Where the Pentacyclic Alkaloid Compounds are administered by injection, an ampule of sterile water for injection or saline can be provided so that the ingredients can be mixed prior to administration.

In one embodiment, the Pentacyclic Alkaloid Compounds are administered intravenously.

The Pentacyclic Alkaloid Compounds can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Pentacyclic Alkaloid Compound to treat or prevent a Condition in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Pentacyclic Alkaloid Compound, and can thus reduce the occurrence of adverse side effects.

Controlled- or sustained-release compositions can initially release an amount of a Pentacyclic Alkaloid Compound that promptly produces the desired therapeutic or prophylactic effect, and gradually and continually release other amounts of the Pentacyclic Alkaloid Compound to maintain this level of therapeutic or prophylactic effect over an extended period of time. To maintain a constant level of the Pentacyclic Alkaloid Compound in the body, the Pentacyclic Alkaloid Compound can be released from the dosage form at a rate that will replace the amount of Pentacyclic Alkaloid Compound being metabolized and excreted from the body. Various conditions can stimulate controlled- or sustained-release of an active ingredient, including, but not limited to, changes in pH, changes in temperature, concentration or availability of enzymes, concentration or availability of water, or other physiological conditions or compounds.

The amount of the Pentacyclic Alkaloid Compound that is effective in the treatment or prevention of a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each patient's circumstances in view of, e.g., published clinical studies. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The effective dosage amounts described herein refer to total amounts administered; that is, if more than one Pentacyclic Alkaloid Compound is administered, the effective dosage amounts correspond to the total amount administered.

The Pentacyclic Alkaloid Compounds can be assayed in vitro or in vivo for the desired therapeutic or prophylactic activity prior to use in humans. Animal model systems can be used to demonstrate safety and efficacy.

The present methods for treating or preventing a Condition in a subject in need thereof can further comprise administering an effective amount of an antiemetic agent to the subject being administered a Pentacyclic Alkaloid Compound.

Examples of useful antiemetic agents include, but are not limited to, metoclopromide, domperidone, prochlorperazine, promethazine, chlorpromazine, trimethobenzamide, ondansetron, granisetron, hydroxyzine, acetyleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxypermdyl, pipamazine, scopolamine, sulpiride, tetrahydrocannabinol, thiethylperazine, thioproperazine, tropisetron, and mixtures thereof.

### 4.25 Kits

The invention encompasses kits that can simplify the administration of a Pentacyclic Alkaloid Compound to a subject.

A typical kit of the invention comprises a unit dosage form of a Pentacyclic Alkaloid Compound. In one embodiment the unit dosage form is a container, which can be sterile, containing an effective amount of a Pentacyclic Alkaloid Compound and a physiologically acceptable carrier or vehicle. The kit can further comprise a label or printed instructions instructing the use of the Pentacyclic Alkaloid Compound to treat or prevent a Condition.

In one embodiment, the kit can further comprise a unit dosage form of another anticancer agent, for example, a container containing an effective amount of the other anticancer agent. In one embodiment the kit comprises a container containing an effective amount of a Pentacyclic Alkaloid Compound and an effective amount of another anticancer agent. Examples of other anticancer agents include, but are not limited to, those listed above.

In another embodiment, the kit can further comprise a unit dosage form of another anticancer agent, for example, a container containing an effective amount of the other therapeutic agent. In one embodiment the kit comprises a container containing an effective amount of a Pentacyclic Alkaloid Compound and an effective amount of another anticancer agent. Examples of other therapeutic agents include, but are not limited to, those listed above.

Kits of the invention can further comprise a device that is useful for administering the unit dosage forms. Examples of such a device includes, but are not limited to, a syringe, a drip bag, a patch, an inhaler, and an enema bag.

### 5. EXAMPLES

General. All non-aqueous reactions were carried out in oven-dried glassware under a slight positive pressure of argon unless otherwise noted. All reagents were commercially available and used without further purification from Sigma-Aldrich (St. Louis, Mo.) or TCI America (Portland, Oreg.), unless indicated otherwise. Solvents were reagent grade and purified using standard techniques: THF was distilled from sodium metal and benzophenone or filtered through a dry-solvent system; CH₂Cl₂ was distilled from calcium hydride or filtered through a dry-solvent system; all other solvents were Aldrich “anhydrous” grade solvents, unless indicated otherwise. Reactions were magnetically stirred and monitored by thin layer chromatography on Merck silica gel 60-F₂₅₄ coated 0.25 mm plates. Preperative thin layer chromatography was performed using Merck silica gel 60-F₂₅₄ coated 0.50 mm plates. Flash chromatography was performed using Sorbent Technology silica gel 60 (particle size 32-63 μm), unless indicated otherwise. Yields reported are for isolated, spectroscopically pure compounds. Melting points are uncorrected. CDCl₃ was allowed to stand over K₂CO₃ and 4 A MS to neutralize and dry prior to NMR sample preparation. NMR spectra were recorded on Bruker DRX 400 or DMX 500 MHz spectrometers. ¹H and ¹³C chemical shifts were referenced to residual solvent peaks. IR spectra were recorded on a Perkin-Elmer Paragon 1000 FTIR spectrometer using sodium chloride plates. High-resolution mass spectra were acquired in the Columbia University Mass Spectral Core facility on a JEOL HX110 spectrometer. Optical rotations were measured on a JASCO DIP-1000 spectrometer.

### 5.1 Example 1

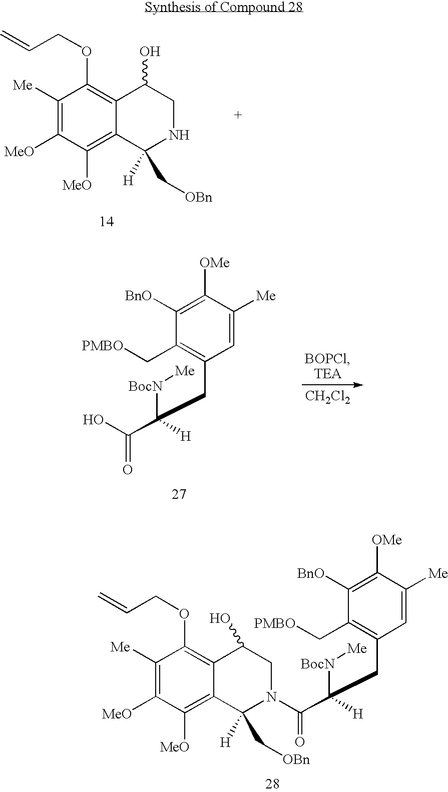

To a solution of Compound 14 (1.85 g, 4.64 mmoles, 1.35 equiv) and Compound 27 (1.99 g, 3.43 mmoles) in CH₂Cl₂ (50 mL) at 0° C. was added TEA (2.39 mL, 17.15 mmoles, 5 equiv) followed by BOPCl (1.31 g, 5.14 mmoles, 1.5 equiv). The resultant reaction was allowed to react for 72 hours to room temperature and was then washed with NaHCO₃, dried over MgSO₄, and concentrated in vacuo. The resultant residue was purified using column chromatography with 50% EtOAc/Hexanes to provide 2.92 g (89%) of Compound 28 as a foam. IR (NaCl) 3418, 2935, 2867, 1685, 1650, 1612, 1585, 1514, 1453, 1414, 1389, 1365, 1319, 1249, 1142, 1121, 1094, 1073, 1032 cm⁻¹; HRMS (FAB) calcd for C₅₆H₆₈N₂O₁₂ [M+H]: 960.48; found 961.4845; [α]_{D}²⁰−51.44 (c=1.0 CHCl₃).

### 5.2 Example 2

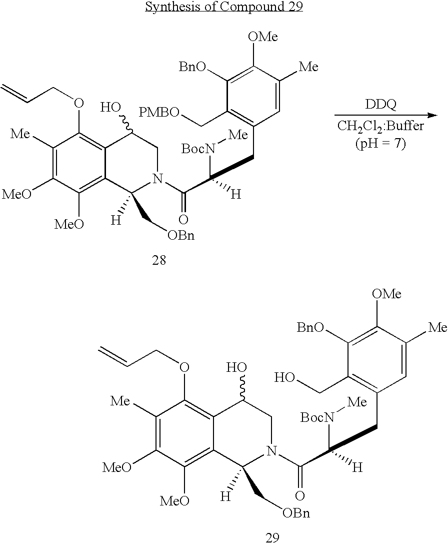

To a solution of Compound 28 (2.20 g, 2.29 mmoles) in (18:1) CH₂Cl₂/Buffer solution pH 7.0 (57 mL) was added DDQ (831 mg, 3.66 mmoles, 1.6 equiv). The resultant reaction was allowed to react for 30 minutes at room temperature and was then washed with NaHCO₃ three times, dried over MgSO₄, and concentrated in vacuo. The resultant residue was purified using column chromatography with 50% EtOAc/Hexanes to provide 1.74 g (90%) of Compound 29 as a foam. IR (NaCl) 3432, 2934, 1650, 1453, 1413, 1391, 1366, 1328, 1235, 1163, 1143, 1121, 1090, 1069, 1009 cm⁻¹; HRMS (FAB) calcd for C₄₉H₆₀N₂O [M+H]: 840.42; found 841.4266; [α]_{D}²⁰−75.93 (c=1.0 CHCl₃).

### 5.3 Example 3

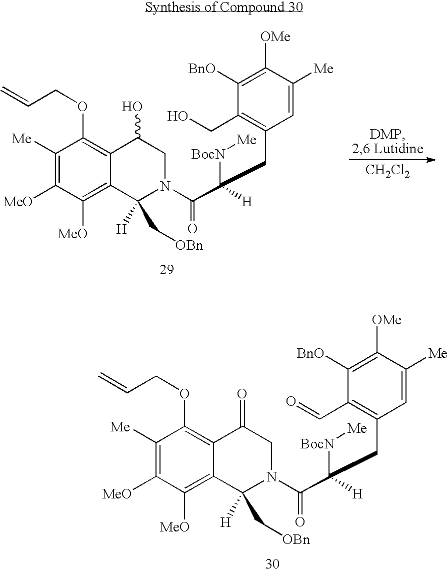

To a solution of Compound 29 (890 mg, 1.05 mmoles) in CH₂Cl₂ (20 mL) at 0° C. was added 2,6 lutidine (0.12 mL, 1.05 mmoles, 1 equiv) followed by DMP (987 mg, 2.32 mmoles, 2.2 equiv). The resultant reaction was allowed to react for 12 hours, during which time it warmed to room temperature. The reaction mixture was quenched using 10% Na₂S₂O₃ in saturated NaHCO₃ and the resultant solution was extracted with CH₂Cl₂ three times. The organic layers were combined, dried over MgSO₄, and concentrated in vacuo. The resultant residue was purified using column chromatography with 50% EtOAc/Hexanes to provide 739 mg (84%) of Compound 30 as a foam. IR (NaCl) 3435, 2935, 2867, 1736, 1688, 1656, 1599, 1578, 1481, 1453, 1411, 1365, 1317, 1236, 1160, 1093, 1058, 1000 cm¹; HRMS (FAB) calcd for C₄₈H₅₆N₂O₁, [M+H]: 836.39; found 837.3950; [α]_{D}²⁰−74.02 (c=1.0 CHCl₃).

### 5.4 Example 4

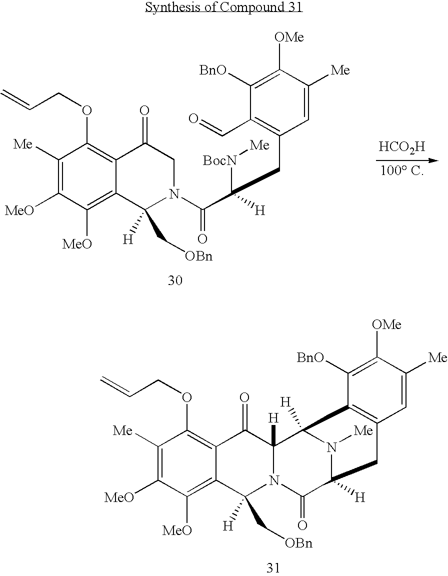

A solution of Compound 30 (106 mg, 0.126 mmoles) in HCO₂H (1.5 mL) was allowed to react for 12 minutes at 100° C. in an oil bath. The resultant reaction was quenched using NaHCO₃ and extracted with CH₂Cl₂ three times. The organic layers were combined, dried over MgSO₄, and concentrated in vacuo. The resultant residue was purified using PTLC with 50% EtOAc/Hexanes to provide 53.3 mg (59%) of Compound 31 as a foam. ¹H NMR (CDCl₃, 500 MHz) δ 7.56 (2H, d, J=7.42 Hz), 7.35 (2H, m), 7.29 (1H, m), 7.11 (3H, m), 6.83 (2H, d, J=7.29 Hz), 6.67 (1H, s), 6.26 (2H, m), 5.41 (1H, d, J=17.10 Hz), 5.29 (1H, d, J=10.40 Hz), 5.13 (1H, d, J=10.51 Hz), 5.12 (1H, s), 5.06 (1H, d, J=10.81 Hz), 4.48 (1H, s), 4.40 (2H, m), 4.16 (1H, d, J=11.74 Hz), 4.01 (1H, d, J=11.75 Hz), 3.86 (3H, s), 3.84 (3H, s), 3.72 (1H, dd, J=3.56 Hz, 10.96 Hz), 3.62 (3H, s), 3.61 (2H, m), 3.13 (1H, dd, J=6.88 Hz, 17.31 Hz), 2.78 (11H, d, J=17.29 Hz), 2.22 (3H, s), 2.19 (3H, s), 2.18 (3H, s) ppm; ¹³C NMR (CDCl₃, 100 MHz) δ 188.7, 169.7, 156.0, 155.2, 149.4, 147.8, 144.4, 137.5, 136.9, 134.2, 132.7, 131.0, 128.9, 128.3, 128.2, 127.8, 126.9, 126.6, 126.0, 125.7, 120.0, 117.5, 74.7, 74.4, 72.6, 69.4, 65.1, 60.6, 60.1, 59.9, 59.2, 51.1, 47.9, 40.3, 29.8, 25.7, 16.0, 9.6 ppm; IR (NaCl) 2933, 1691, 1655, 1575, 1446, 1408, 1335, 1284, 1234, 1174, 1141, 1104, 1069, 1000 cm⁻¹; HRMS (FAB) calcd for C₄₃H₄₆N₂O₈ [M+H]: 718.33; found 719.3352; [α]_{D}²⁰+22.94 (c=1.0 CHCl₃).

### 5.5 Example 5

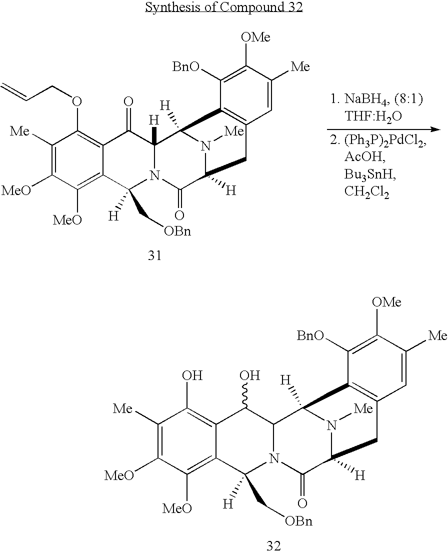

To a solution of Compound 31 (62 mg, 0.0863 mmoles) in (8:1) THF:H₂O (2 mL) at 0° C. was added sodium borohydride (9.5 mg, 0.259 mmoles, 3 equiv). The resultant reaction was allowed to react for 2 hours to room temperature and was then quenched using NH₄Cl and extracted with EtOAc three times. The organic layers were combined, washed with brine, dried over MgSO₄, and concentrated in vacuo. The resultant residue was dissolved in CH₂Cl₂ (3 mL). After cooling to 0° C., acetic acid (49 μL, 0.863 mmoles, 10 equiv), Bu₃SnH (27.8 μL, 0.104 mmoles, 1.2 equiv), and (Ph₃P)₂PdCl₂ (12.1 mg, 0.0173 mmoles, 0.2 equiv) were added. The resultant reaction was allowed to react for 30 minutes to room temperature. The reaction was then concentrated in vacuo and the residue was purified using PTLC with 60% EtOAc/Hexanes to provide 57.4 mg (98%) of Compound 32 as a white solid. ¹H NMR (CDCl₃, 400 MHz) δ 8.04 (1H, bs), 7.35 (5H, m), 7.18 (3H, m), 6.87 (2H, m), 6.60 (1H, s), 5.87 (1H, dd, J=2.97 Hz, 7.14 Hz), 5.11 (1H, m), 5.10 (1H, d, J=10.74 Hz), 5.00 (1H, d, J=10.76 Hz), 4.23 (1H, s), 4.11 (1H, d, J=12.07 Hz), 3.92 (1H, d, J=12.07 Hz), 3.77 (3H, s), 3.75 (3H, s), 3.72 (4H, m), 3.63 (2H, m), 3.56 (2H, m), 3.10 (1H, dd, J=6.61 Hz, 17.54 Hz), 2.71 (1H, d, J=17.49 Hz), 2.25 (3H, s), 2.17 (3H, s), 2.13 (3H, s) ppm; ¹³C NMR (CDCl₃, 125 MHz) δ 169.5, 151.4, 149.3, 148.8, 142.2, 138.1, 137.1, 131.3, 129.0, 128.8, 128.6, 128.5, 128.0, 127.1, 126.8, 126.0, 124.7, 124.5, 118.8, 117.3, 74.9, 72.8, 71.1, 67.9, 61.1, 60.4, 60.0, 59.9, 58.9, 50.9, 48.7, 39.2, 29.7, 23.7, 15.8, 8.7 ppm; IR (NaCl) 3271, 2931, 2854, 1620, 1585, 1455, 1413, 1335, 1293, 1260, 1228, 1205, 1177, 1125, 1082, 1065, 1024 cm⁻¹; HRMS (FAB) calcd for C₄₀H₄₄N₂O₈ [M+H]: 680.31; found 681.3173; [α]_{D}²¹−1.23 (c=1.0 CHCl₃).

### 5.6 Example 6

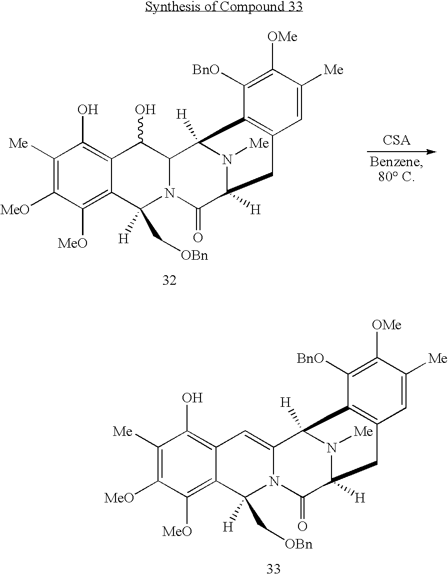

To a solution of Compound 32 (90 mg, 0.132 mmoles) in benzene (4 mL) was added camphorsulfonic acid (92 mg, 0.397 mmoles, 3 equiv). The resultant reaction was allowed to react for 4 hours at 80° C. in an oil bath and was then quenched using NaHCO₃ and extracted with CH₂Cl₂ three times. The organic layers were combined, washed with brine, dried over MgSO₄, and concentrated in vacuo. The resultant residue was purified using PTLC with 60% EtOAc/Hexanes to provide 70 mg (80%) of Compound 33 as a white solid. ¹H NMR (CDCl₃, 400 MHz) δ 7.45 (5H, m), 7.25 (3H, m), 6.95 (2H, m), 6.42 (1H, s), 6.25 (1H, dd, J=3.42 Hz, 8.21 Hz), 5.97 (1H, s), 5.21 (1H, d, J=11.06 Hz), 4.98 (1H, d, J=11.09 Hz), 4.58 (1H, s), 3.95 (1H, d, J=12.32 Hz), 3.82 (3H, s), 3.77 (3H, s), 3.75 (3H, s), 3.74 (2H, m), 3.63 (1H, m), 3.24 (2H, m), 3.09 (1H, dd, J=8.85 Hz, 9.80 Hz), 3.00 (1H, d, J=16.70 Hz), 2.48 (3H, s), 2.12 (6H, s) ppm; ¹³C NMR (CDCl₃, 100 MHz) δ 150.0, 149.1, 148.2, 144.4, 143.0, 138.3, 138.0, 132.5, 131.2, 128.4, 128.0, 127.7, 126.7, 126.6, 126.4, 126.0, 125.5, 120.4, 118.0, 113.9, 101.5, 88.4, 76.7, 73.7, 72.4, 70.7, 60.9, 60.8, 60.2, 56.7, 54.5, 46.9, 41.8, 33.1, 15.9, 9.0 ppm; IR (NaCl) 3341, 2938, 2855, 2248, 1664, 1634, 1580, 1463, 1425, 1355, 1323, 1234, 1183, 1126, 1060, 1002 cm⁻¹; HRMS (FAB) calcd for C₄₀H₄₂N₂O₇ [M+H]: 662.30; found 663.3052; [α]_{D}²¹−73.57 (c=1.0 CHCl₃).

### 5.7 Example 7

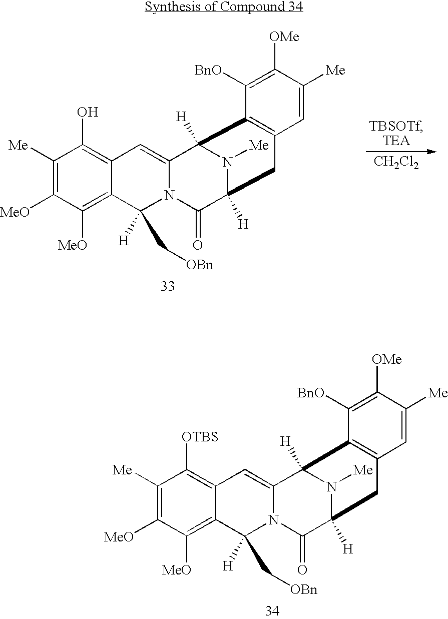

To a solution of Compound 33 (57.4 mg, 0.0867 mmoles) in CH₂Cl₂ (4 mL) at 0° C. was added TEA (0.12 mL, 0.867 mmoles, 10 equiv) and TBSOTf (59.7 μL, 0.260 mmoles, 3 equiv). The resultant reaction was then allowed to warm to room temperature with stirring over 30 minutes. The reaction was quenched using MeOH at 0° C. and the resultant solution was concentrated in vacuo. The resultant residue was purified using PTLC with 40% EtOAc/Hexanes to provide 60.4 mg (90%) of Compound 34 as an off-white foam. ¹H NMR (CDCl₃, 400 MHz) δ 7.30 (8H, m), 7.01 (2H, m), 6.33 (1H, s), 6.26 (1H, dd, J=3.55 Hz, 8.98 Hz), 6.13 (1H, s), 5.13 (1H, d, J=11.05 Hz), 5.01 (1H, d, J=11.09 Hz), 4.44 (1H, s), 3.93 (1H, d, J=12.27 Hz), 3.83 (3H, s), 3.75 (3H, s), 3.73 (3H, s), 3.70 (2H, m), 3.59 (1H, m), 3.2 (2H, m), 2.98 (2H, m), 2.41 (3H, s), 2.12, (3H, s), 2.08 (3H, s), 0.99 (9H, s), 0.14 (3H, s), 0.11 (3H, s) ppm; ¹³C NMR (CDCl₃, 100 MHz) δ 168.0, 150.3, 148.9, 148.1, 144.7, 143.5, 138.4, 137.4, 131.8, 131.1, 128.3, 128.2, 128.0, 127.8, 127.7, 126.9, 126.6, 126.3, 126.0, 122.9, 120.8, 118.4, 104.5, 74.7, 72.3, 70.5, 61.0, 60.7, 60.3, 59.9, 56.8, 47.0, 42.3, 33.4, 29.8, 26.2, 18.7, 15.9, 11.1, −2.7, −3.5 ppm; IR (NaCl) 2933, 2856, 2360, 1677, 1638, 1463, 1419, 1359, 1239, 1127, 1069, 1002 cm⁻¹; HRMS (FAB) calcd for C₄₆H₅₆N₂O₇Si [M+H]: 776.39; found 777.3939; [α]_{D}²¹−22.81 (c=1.0 CHCl₃).

### 5.8 Example 8

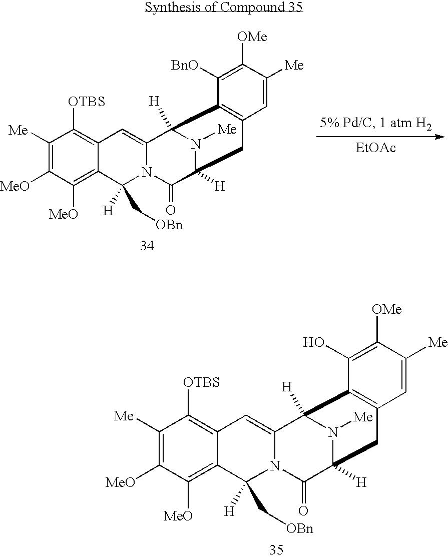

To a solution of Compound 34 (27 mg, 0.0347 mmoles) in EtOAc (5 mL) was added 5% Pd/C (2.7 mg, 10% w/w) and 1 atm of H₂. The suspension was allowed to react for 24 hours at room temperature. The reaction was filtered and concentrated in vacuo. The resultant residue was purified using PTLC with 50% EtOAc/Hexanes to provide 21.4 mg (90%) of Compound 35 as a clear film. ¹H NMR (CDCl₃, 400 MHz) δ 7.24 (4H, m), 7.00 (2H, m), 6.27 (1H, dd, J=3.63 Hz, 12.64 Hz), 6.12 (1H, s), 5.75 (1H, bs), 4.55 (1H, s), 3.89 (1H, d, J=12.25 Hz), 3.84 (3H, s), 3.75 (3H, s), 3.67 (3H, s), 3.65 (2H, m), 3.25 (1H, dd, J=6.60 Hz, 16.55 Hz), 3.14 (1H, dd, J=3.73 Hz, 10.06 Hz), 3.01 (2H, m), 2.54 (3H, s), 2.12 (3H, s), 2.08 (3H, s), 1.12 (9H, s), 0.21 (3H, s), 0.20 (3H, s) ppm; ¹³C NMR (CDCl₃, 100 MHz) δ 167.8, 150.2, 144.9, 144.3, 143.6, 142.9, 138.5, 130.9, 128.8, 128.2, 127.6, 126.5, 126.3, 122.9, 122.0, 120.3, 119.5, 118.4, 103.9, 72.7, 70.5, 61.1, 60.7, 60.0, 56.4, 46.9, 42.1, 33.4, 29.8, 26.2, 18.8, 15.8, 10.9, −2.9, −3.3 ppm; IR (NaCl) 3368, 2931, 2856, 1672, 1638, 1462, 1420, 1360, 1238, 1127, 1069 cm⁻¹; HRMS (FAB) calcd for C₃₉H₅₀N₂O₇Si [M+H]: 686.34; found 687.3474; [α]_{D}²¹−19.17 (c=1.0 CHCl₃).

### 5.9 Example 9

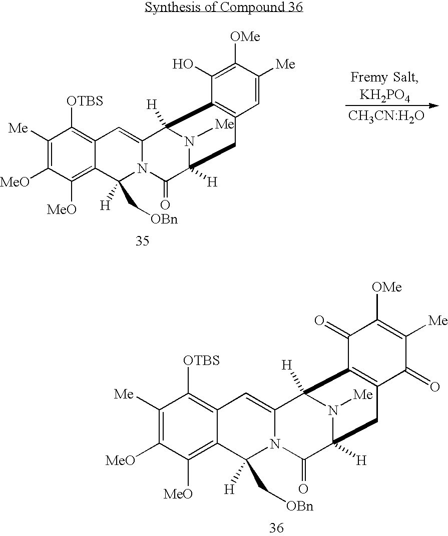

To a solution of Compound 35 (34.0 mg, 0.0496 mmoles) in CH₃CN (3 mL) was added a H₂O (2 mL) solution of Fremy's salt (39.9 mg, 0.149 mmoles, 3 equiv) and KH₂PO₄ (40.5 mg, 0.298 mmoles, 6 equiv). The resultant reaction was allowed to react for 36 hours at room temperature, then diluted with brine and extracted with EtOAc three times. The organic layers were combined, dried over MgSO₄, and concentrated in vacuo. The resultant residue was purified using PTLC with 45% EtOAc/Hexanes to provide 29.1 mg (84%) of Compound 36 as a red film. ¹H NMR (CDCl₃, 400 MHz) δ 7.24 (3H, m), 7.03 (2H, m), 6.27 (1H, dd, J=4.38 Hz, 8.20 Hz), 6.25 (1H, s), 4.43 (1H, s), 4.39 (1H, d, J=12.02 Hz), 4.18 (1H, d, J=12.00 Hz), 3.89 (3H, s), 3.82 (3H, s), 3.76 (3H, s), 3.64 (1H, m), 3.49 (1H, s), 3.25 (2H, m), 2.89 (2H, m), 2.52 (3H, s), 2.12 (3H, s), 1.80 (3H, s), 1.12 (9H, s), 0.22 (3H, s), 0.20 (3H, s) ppm; ¹³C NMR (CDCl₃, 100 MHz) δ 186.3, 180.3, 166.6, 154.9, 150.8, 144.7, 143.5, 139.2, 137.7, 136.9, 128.5, 128.4, 128.0, 127.1, 126.6, 123.1, 120.4, 118.1, 106.3, 72.6, 70.1, 60.8, 60.7, 59.9, 59.8, 54.7, 46.3, 41.6, 29.5, 26.1, 18.8, 10.9, −2.8, −3.4 ppm; IR (NaCl) 2933, 2857, 1675, 1655, 1616, 1463, 1419, 1360, 1293, 1276, 1234, 1129, 1071 cm⁻¹; HRMS (FAB) calcd for C₃₉H₄₈N₂O₈Si [M+H]: 700.32; found 703.3428; [α]_{D}²⁵−29.18 (c=1.0 CHCl₃).

### 5.10 Example 10

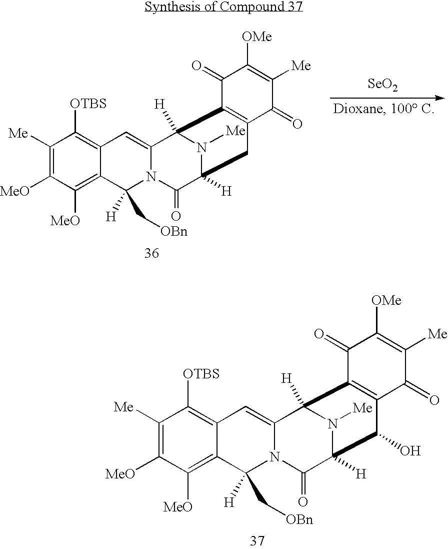

To a solution of Compound 36 (24.0 mg, 0.0342 mmoles) in dioxane (3 mL) was added SeO₂ (13.3 mg, 0.119 mmoles, 3.5 equiv). The resultant reaction was heated in a vial for 5 hours at 100° C. in an oil bath. The reaction mixture was then concentrated in vacuo. The resultant residue was purified using PTLC with 50% EtOAc/Hexanes to provide 21.3 mg (87%) of Compound 37 as a red film. ¹H NMR (CDCl₃, 400 MHz) δ 7.24 (3H, m), 7.01 (2H, m), 6.29 (1H, s), 6.21 (1H, d, J=6.08 Hz), 4.84 (1H, bs), 4.42 (1H, s), 4.37 (1H, d, J=12.01 Hz), 4.14 (1H, d, J=12.00 Hz), 3.88 (3H, s), 3.80 (3H, s), 3.75 (3H, s), 3.68 (1H, m), 3.21 (2H, m), 2.87 (1H, m), 2.57 (3H, s), 2.10 (3H, s), 1.80 (3H, s), 1.12 (9H, s), 0.21 (3H, s), 0.20 (3H, s) ppm; ¹³C NMR (CDCl₃, 100 MHz) δ 186.2, 180.8, 163.4, 155.1, 151.0, 144.9, 143.4, 137.8, 137.6, 137.5, 128.9, 128.0, 127.2, 126.7, 123.3, 120.7, 117.9, 107.5, 72.6, 69.9, 67.2, 65.2, 60.9, 60.7, 59.9, 55.2, 46.3, 41.9, 29.8, 26.2, 18.8, 10.9, 8.7, −2.8, −3.4 ppm; IR (NaCl) 3292, 2932, 2857, 1658, 1617, 1462, 1420, 1357, 1304, 1234, 1128, 1071 cm⁻¹; HRMS (FAB) calcd for C₃₉H₄₈N₂O₉Si [M+H]: 716.31; found 719.3383; [α]_{D}²¹−67.50 (c=1.0 CHCl₃).

### 5.11 Example 11

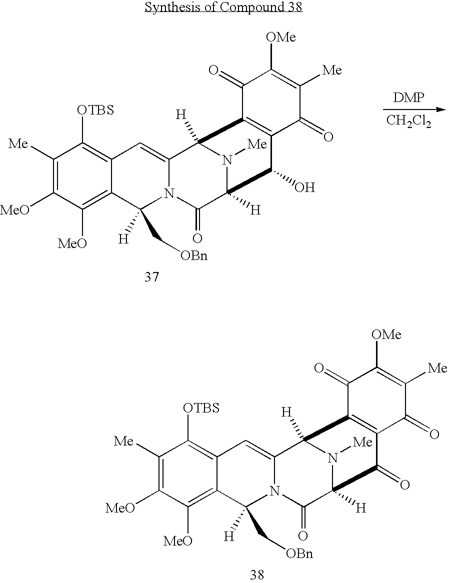

To a solution of Compound 37 (18.4 mg, 0.0257 mmoles) in CH₂Cl₂ was added Dess-Martin Periodinane (16.3 mg, 0.0385 mmoles, 1.5 equiv). The resultant reaction was allowed to react for 12 hours at room temperature, then was quenched using 10% Na₂S₂O₃ and extracted with CH₂Cl₂ three times. The organic layers were combined, dried over MgSO₄, and concentrated in vacuo. The resultant residue was purified using PTLC to provide 18.0 mg (99%) of Compound 38 as a purple film. ¹H NMR (CDCl₃, 400 MHz) δ 7.22 (3H, m), 6.96 (2H, m), 6.49 (1H, s), 6.39 (1H, dd, J=4.36 Hz, 7.40 Hz), 4.69 (1H, s), 4.42 (1H, d, J=12.14 Hz), 4.1.2 (1H, d, J=12.10 Hz), 3.99 (1H, s), 3.88 (3H, s), 3.79 (3H, s), 3.75 (3H, s), 3.25 (2H, m), 2.57 (3H, s), 2.10 (3H, s), 1.80 (3H, s), 1.12 (9H, s), 0.21 (3H, s), 0.20 (3H, s) ppm; ¹³C NMR (CDCl₃, 100 MHz) δ 185.1, 183.1, 181.7, 159.3, 154.6, 151.4, 146.6, 145.1, 143.5, 137.4, 129.9, 128.1, 127.2, 126.9, 125.1, 124.9, 123.4, 120.8, 117.6, 108.7, 73.2, 72.7, 69.8, 60.7, 60.6, 59.9, 46.9, 41.7, 29.8, 26.1, 18.8, 10.9, 8.9, −2.8, −3.4 ppm; IR (NaCl) 2931, 2857, 1724, 1664, 1637, 1600, 1463, 1419, 1350, 1294, 1258, 1226, 1203, 1127, 1072 cm⁻¹; HRMS (FAB) calcd for C₃₉H₄₆N₂O₉Si [M+H]: 714.30; found 717.3236; [α]_{D}²¹−133.01 (c=1.0 CHCl₃).

### 5.12 Example 12

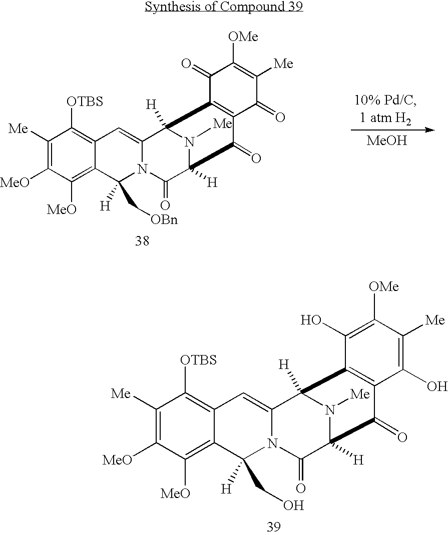

To a solution of Compound 38 (12.7 mg, 0.0177 mmoles) in MeOH (2 mL) was added 10% Pd/C (4 mg, 30% w/w) under 1 atmosphere of H₂. The resultant suspension was stirred vigorously for 24 hours at room temperature and the reaction mixture was then filtered and concentrated in vacuo. The resultant residue was purified using PTLC with 65% EtOAc/Hexanes to provide 10.0 mg (90%) of Compound 39 as a yellow film. ¹H NMR (CDCl₃, 400 MHz) δ 11.42 (1H, s), 7.33 (1H, s), 6.13 (1H, dd, J=4.84 Hz, 8.00 Hz), 5.51 (1H, s), 4.77 (1H, s), 4.07 (1H, s), 3.89 (3H, s), 3.84 (3H, s), 3.77 (3H, s), 3.39 (1H, m), 3.20 (1H, m), 2.59 (3H, s), 2.20 (3H, s), 2.11 (3H, s), 1.12 (9H, s), 0.21 (3H, s), 0.20 (3H, s) ppm; ¹³C NMR (CDCl₃, 100 MHz) δ 193.4, 161.5, 155.9, 152.6, 150.8, 144.8, 143.7, 137.4, 128.2, 123.4, 122.1, 119.8, 118.4, 117.4, 108.8, 105.1, 72.5, 64.7, 61.2, 60.7, 59.9, 56.5, 50.0, 41.6, 29.8, 26.1, 18.8, 10.9, 9.1, −2.9, −3.3 ppm; IR (NaCl) 3401, 2933, 2858, 1678, 1649, 1591, 1464, 1419, 1378, 1295, 1248, 1205, 1172, 1122, 1071, 1005 cm⁻¹; HRMS (FAB) calcd for C₃₂H₄₂N₂O₉Si [M+H]: 626.27; found 627.2714; [α]_{D}²⁵+262.26 (c=1.0 CHCl₃).

### 5.13 Example 13

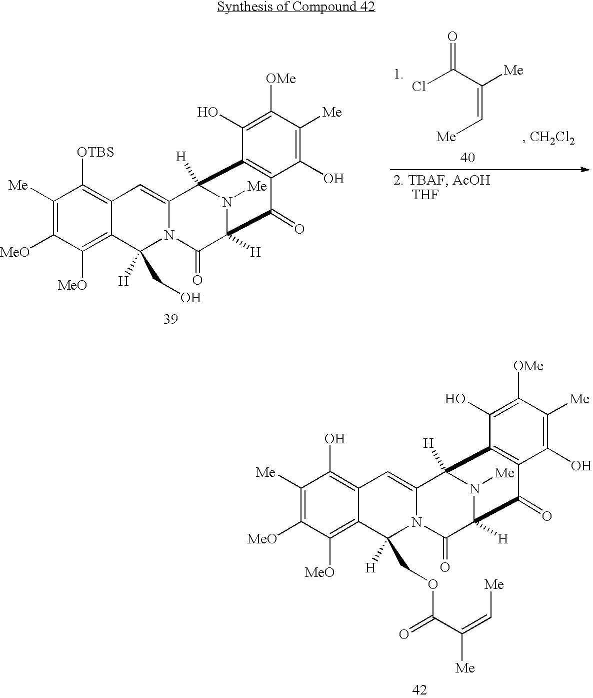

To a solution of Compound 39 (10.0 mg, 0.0159 mmoles) in CH₂Cl₂ (1.5 mL) was added a solution of Compound 40 (47.1 mg, 0.397 mmoles, 25 equiv) in CH₂Cl₂ (1 mL). The resultant solution was allowed to age for 12 hours at room temperature, concentrated in vacuo, and the resultant residue was dissolved in THF (1 mL). The resultant solution was cooled to 0° C., and acetic acid (18 μL, 0.318 mmoles, 20 equiv) was added, followed by tetra-n-butylammonium fluoride (1.0 M in THF, 40 μL, 0.0399 mmoles, 2.5 equiv). The resultant reaction was allowed to react for 2 hours to room temperature. The reaction was concentrated in vacuo and the resultant residue was purified using PTLC to provide 7.0 mg (75%) of Compound 42 as a yellow film. ¹H MR(CDCl₃, 400 MHz) δ 11.44 (1H, s), 6.42 (1H, s), 6.39 (1H, dd, J=3.18 Hz, 9.38 Hz), 5.96 (1H, dd, J=5.99 Hz, 13.24 Hz), 5.72 (1H, s), 4.93 (1H, s), 4.80 (1H, s), 4.06 (1H, s), 3.87 (3H, s), 3.86 (1H, m), 3.83 (3H, s), 3.78 (3H, s), 3.70 (1H, dd, J=3.23 Hz, 11.40 Hz), 2.60 (3H, s), 2.16 (3H, s), 2.12 (3H, s), 1.79 (3H, dd, J=1.43 Hz, 7.25 Hz), 1.54 (3H, s) ppm; ¹³C NMR (CDCl₃, 100 MHz) δ 193.3, 166.7, 160.5, 155.6, 152.7, 150.5, 144.9, 143.1, 138.6, 137.7, 128.5, 126.7, 121.8, 119.5, 118.2, 118.0, 113.6, 108.9, 103.2, 72.6, 62.7, 61.2, 60.9, 60.4, 56.7, 46.3, 41.5, 20.1, 15.7, 9.1, 8.9 ppm; IR (NaCl) 3425, 2929, 1677, 1646, 1463, 1417, 1381, 1355, 1292, 1230, 1150, 1120, 1068, 1047 cm⁻¹; HRMS (FAB) calcd for C₃₁H₃₄N₂O₁₀ [M+H]: 594.22; found 595.2270; [α]_{D}²⁰+68.24 (c=0.5 CHCl₃).

### 5.14 Example 14

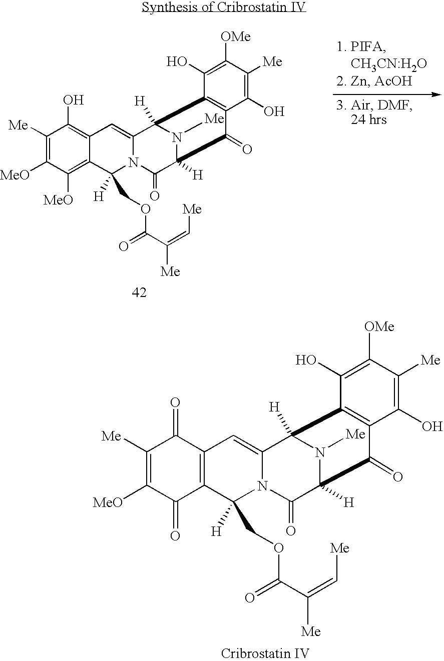

To a solution of phenol 42 (4.0 mg, 0.00673 mmoles) in (2:1) CH₃CN/H₂O (1 mL) was added Phenyliodine (III) bis(trifluoroacetate) (6.4 mg, 0.0148 mmoles, 2.2 equiv). The resultant reaction was allowed to react for 10 minutes at room temperature and was then quenched using NaHCO₃ and extracted with CH₂Cl₂ three times. The organic layers were combined, dried over MgSO₄, and concentrated in vacuo. The resultant deep red residue was dissolved in acetic acid (0.8 mL), and to it was added zinc dust (9.8 mg, 0.138 mmoles, 20 equiv). The resultant reaction was allowed to react for 10 minutes at room temperature and was then filtered, quenched using NaHCO₃, and extracted with CH₂Cl₂ three times. The organic layers were combined, dried over MgSO₄, and concentrated in vacuo. The resultant yellow residue was dissolved in DMF (0.8 mL) and air was passed through for 5 minutes. The resultant reaction was allowed to react (air atmosphere) for 12 hours at room temperature. The reaction mixture was then concentrated in vacuo, and the residue was purified using column chromatograpy (Sephadex LH-20 stationary phase, eluent 100% MeOH) to provide 2.5 mg (65%) of Cribrostatin IV as a red film. ¹H NMR (CDCl₃, 400 MHz) δ 11.35 (1H, s), 6.27 (1H, s), 6.21 (1H, dd, J=3.08 Hz, 6.04 Hz), 5.92 (1H, dd, J=1.41 Hz, 7.24 Hz), 5.57 (1H, s), 4.88 (1H, s), 4.12 (1H, s), 4.07 (1H, m), 4.06 (3H, s), 3.87 (3H, s), 3.83 (1H, m), 2.57 (3H, s), 2.17 (3H, s), 1.96 (3H, s), 1.75 (3H, dd, J=1.47 Hz, 7.24 Hz), 1.48 (3H, m) ppm; ¹³C NMR (CDCl₃, 100 MHZ) δ ppm; IR (NaCl) cm⁻¹; HRMS (FAB) calcd for C₃₀H₃₀N₂O₁₀ [M+H]: 578.19; found 579.1968; [α]_{D}²⁰ (c=0.5 CHCl₃).

### 5.15 Example 15

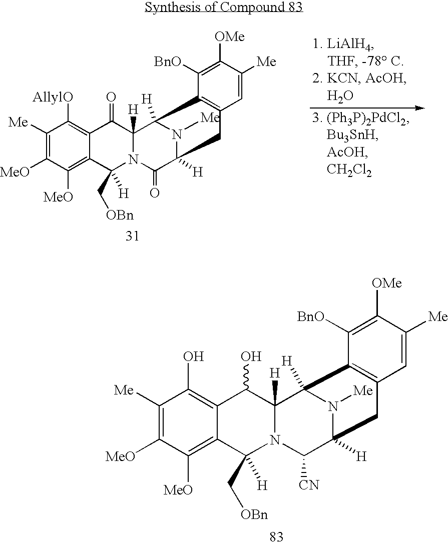

To solution of Compound 31 (67.0 mg, 0.0933 mmoles) in 15 mL of THF, at −78° C., was added, dropwise, a 1.0 M solution of lithium aluminumhydride (2.80 mL, 2.80 mmoles, 30 equiv) in THF. The reaction solution was allowed to warm to room temperature over 12 hours. The reaction was cooled to −78° C., followed by careful addition of potassium cyanide (69.0 mg, 1.41 mmoles, 15 equiv) in 3.0 mL of H₂O. The cooling bath was removed, followed by addition of AcOH (0.11 mL, 1.77 mmoles, 20 equiv) and 1.5 mL of THF. The resultant reaction slurry allowed to warm to room temperature over 12 hours. The reaction mixture was then quenched with a saturated solution of NaHCO₃ and extracted with CH₂Cl₂ three times. The organic layers were combined, dried with MgSO₄, and concentrated by rotary evaporation. The resultant crude residue was dissolved in 5 mL of CH₂Cl₂. To this solution, at room temperature, was added tributyltinhydride (38 μL, 0.121 mmoles, 1.5 equiv), AcOH (53 μL, 0.936 mmoles, 10 equiv), and (Ph₃P)₂PdCl₂ (13.0 mg, 0.185 mmoles, 0.2 equiv). The solution was allowed to react for 15 minutes and then concentrated by rotary evaporation. The resultant residue was purified by column chromatography with 35% EtOAc/Hexanes to provide 44.0 mg (69%) of Compound 83 as an off-white foam: ¹H NMR (CDCl₃, 400 MHz) δ 7.94 (1H, s), 7.43-7.32 (5H, m), 7.26 (3H, m), 6.98 (2H, m), 6.58 (1H, s), 5.58 (1H, t, J=9.77 Hz), 5.09 (1H, d, J=11.02 Hz), 5.05 (1H, d, J=11.02 Hz), 4.15 (1H, s), 3.99 (1H, dd, J=2.54 Hz, 8.50 Hz), 3.85 (3H, m), 3.77 (3H, s), 3.76 (3H, s), 3.62 (1H, dd, J=2.76 Hz, 11.25 Hz), 3.31-3.05 (4H, m), 2.54 (1H, d, J=17.92 Hz), 2.27 (3H, s), 2.14 (3H, s), 2.13 (3H, s), 1.44 (1H, d, J=10.00 Hz) ppm; ¹³C NMR (CDCl₃, 100 MHz) δ 151.1, 148.8, 148.2, 142.1, 138.5, 137.1, 130.5, 129.6, 128.7, 128.6, 128.4, 128.0, 127.1, 126.9, 125.9, 124.8, 123.6, 119.9, 118.6, 117.2, 75.1, 73.7, 71.6, 63.1, 61.4, 60.5, 60.1, 60.0, 58.8, 58.2, 55.8, 41.8, 25.3, 15.9, 8.9 ppm; IR (NaCl) 3373, 2931, 2360, 1455, 1414, 1121, 1066, 1013 cm¹; HRMS (FAB) calcd for C₄₁H₄₅N₃O₇ [M+H]: 691.33; found 692.3310; [α]_{D}¹⁷+11.21 (c=1.0 CHCl₃).

### 5.16 Example 16

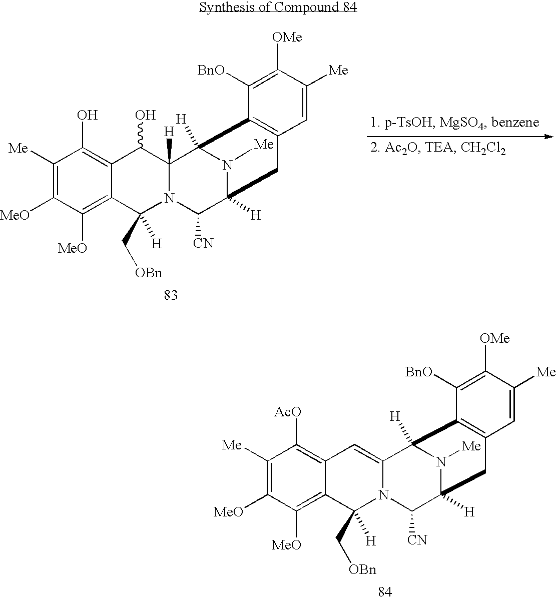

To solution of Compound 83 (15.0 mg, 0.0217 mmoles) and MgSO₄ (11.0 mg, 0.0868 mmoles, 4 equiv) in 1.5 mL of benzene, at 25° C., was added, TsOH (61.8 mg, 0.325 mmoles, 15 equiv). The solution was kept for 4 hours at room temperature. The reaction solution was then diluted with H₂O and extracted with CH₂Cl₂ three times. The organic layers were combined, dried with MgSO₄, and concentrated by rotary evaporation. The resultant crude residue was dissolved in 1.5 mL of CH₂Cl₂. To this solution, at room temperature, was added acetic anhydride (20 μL, 0.217 mmoles, 10 equiv) and TEA (30 μL, 0.217 mmoles, 10 equiv). The solution was kept for 5 hours at room temperature. The reaction solution was then quenched with an aqueous solution of NaHCO₃ and extracted with CH₂Cl₂ three times. The resultant residue was purified by PTLC with 35% EtOAc/Hexanes to provide 13.8 mg (90%) of Compound 84 as a yellowish foam: ¹H NMR (CDCl₃, 400 MHz) δ 7.50 (2H, d, J=7.51 Hz), 7.42 (2H, t, J=7.37 Hz), 7.37-7.26 (4H, m), 7.13 (2H, d, J=7.33 Hz), 6.43 (1H, s), 5.62 (1H, s), 5.11 (1H, d, J=11.27 Hz), 5.05 (1H, d, J=11.27 Hz), 4.73 (1H, d, J=7.47 Hz), 4.56 (1H, s), 4.21 (1H, s), 3.98 (2H, dd, J=12.18 Hz, 15.65 Hz), 3.83 (3H, s), 3.79 (3H, s), 3.74 (3H, s), 3.45 (1H, d, J=7.75 Hz), 3.25-2.95 (3H, m), 2.66 (1H, d, J=17.80 Hz), 2.40 (3H, s), 2.20 (3H, s), 1.98 (3H, s), 1.95 (3H, s) ppm; ¹³C NMR (CDCl₃, 100 MHz) δ 168.4, 148.9, 148.5, 146.2, 139.0, 138.2, 137.9, 130.5, 128.4, 128.1, 127.7, 127.6, 127.5, 127.2, 126.7, 125.0, 124.5, 121.1, 119.1, 117.1, 96.1, 74.1, 73.0, 72.7, 60.7, 60.5, 60.2, 57.6, 57.2, 56.6, 56.0, 41.6, 29.8, 27.6, 20.2, 16.0, 9.9 ppm; IR (NaCl) 2929, 2852, 2360, 1762, 1685, 1453, 1350, 1205, 1064, 1007 cm⁻¹; HRMS (FAB) calcd for C₄₃H₄₅N₃O₇ [M+H]: 715.33; found 716.3328; [α]_{D}¹⁷+95.92 (c=1.0 CHCl₃).

### 5.17 Example 17

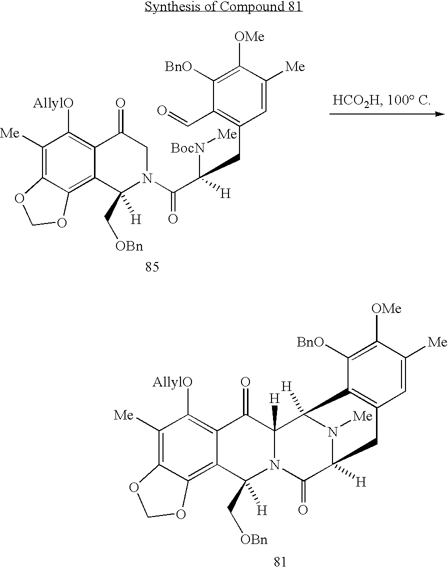

Compound 85 (100 mg, 0.122 mmoles) was kept in 1 mL of formic acid at 100° C., in an oil bath, for 15 minutes. The reaction was then cooled, quenched with a saturated aqueous solution of NaHCO₃, and extracted with CH₂Cl₂ three times. The organic layers were combined, dried with MgSO₄, and concentrated by rotary evaporation. The resultant residue was purified by PTLC with 50% EtOAc/Hexanes to provide 51.1 mg (60%) of Compound 81 as an off-white foam: ¹H NMR (CDCl₃, 400 Hz) δ 7.55 (2H, d, J=7.20 Hz), 7.42-7.30 (5H, m), 7.14 (3H, m), 6.84 (2H, d, J=7.14 Hz), 6.70 (1H, s), 6.28 (1H, m), 6.11 (1H, t, J=4.58 Hz), 5.99 (2H, s), 5.45 (1H, d, J=17.26 Hz), 5.33 (1H, d, J=10.26 Hz), 5.21 (1H, s), 5.17 (1H, d, J=10.75 Hz), 5.08 (1H, d, J=10.75 Hz), 4.58 (1H, s), 4.51 (1H, dd, J=5.73 Hz, 11.70 Hz), 4.49 (1H, dd, J=5.72 Hz, 11.69 Hz), 4.18 (1H, d, J=12.06 Hz), 4.06 (1H, d, J=12.07 Hz), 3.77-3.61 (3H, m), 3.64 (3H, s), 3.16 (1H, dd, J=6.82 Hz, 17.40 Hz), 2.82 (1H, d, J=17.36 Hz), 2.29 (3H, s), 2.22 (3H, s), 2.19 (3H, s) ppm; ¹³C NMR (CDCl₃, 100 MHz) δ 187.1, 169.6, 155.8, 150.3, 149.4, 147.9, 138.6, 137.5, 137.0, 134.1, 131.0, 128.8, 128.2, 128.2, 127.9, 127.0, 126.7, 126.1, 125.7, 118.5, 117.7, 114.4, 102.0, 75.0, 74.3, 72.9, 69.2, 65.5, 59.9, 59.1, 50.3, 48.0, 40.2, 25.7, 16.0, 9.3 ppm; IR (NaCl) 2930, 2857, 1687, 1654, 1451, 1337, 1286, 1117 cm¹¹; HRMS (FAB) calcd for C₄₂H₄₂N₂O₈ [M+H]: 702.29; found 703.2999; [α]_{D}¹⁷+35.21 (c=1.0 CHCl₃).

### 5.18 Example 18

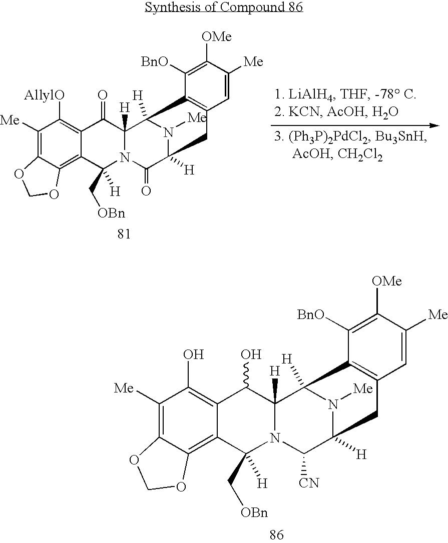

To solution of Compound 81 (84.0 mg, 0.119 mmoles) in 12 mL of THF, at −78° C., was added, dropwise, a 1.0 M THF solution of lithium aluminumhydride (3.00 mL, 2.99 mmoles, 25 equiv). The solution was allowed to react while warming to room temperature over 12 hours. The solution was then cooled to −78° C., followed by carefuil addition of potassium cyanide (87.0 mg, 1.78 mmoles, 15 equiv) in 3.8 mL of H₂O. The cooling bath was removed, followed by addition of AcOH (0.13 mL, 2.38 mmoles, 20 equiv) and 3 mL of THF. The resultant slurry was allowed to react while warming to room temperature over 12 hours. The reaction mixture was then quenched with a saturated solution of NaHCO₃ and extracted with CH₂Cl₂ three times. The organic layers were combined, dried with MgSO₄, and concentrated by rotary evaporation. The resultant crude residue was dissolved in 5 mL of CH₂Cl₂. To this solution, at room temperature, was added tributyltinhydride (48 μL, 0.179 mmoles, 1.5 equiv), AcOH (67 μL, 1.19 mmoles, 10 equiv), and (Ph₃P)₂PdCl₂ (16.7 mg, 0.0238 mmoles, 0.2 equiv). The solution was allowed to react for 15 minutes and then the reaction was concentrated by rotary evaporation. The resultant residue was purified by column chromatography with 35% EtOAc/Hexanes to provide 50.0 mg (62%) of Compound 86 as an off-white foam: ¹H NMR (CDCl₃, 400 MHz) δ 7.94 (1H, s), 7.41-7.30 (5H, m), 7.26 (3H, m), 6.96 (2H, m), 6.57 (1H, s), 5.83 (2H, d, J=1.98 Hz), 5.58 (1H, t, J=10.10 Hz), 5.09 (1H, d, J=10.92 Hz), 5.02 (1H, d, J=10.92 Hz), 4.28 (1H, s), 3.92 (1H, d, J=12.10 Hz), 3.88 (1H, d, J=2.48 Hz), 3.82 (1H, d, J=12.07 Hz), 3.79 (1H, d, J=1.57 Hz), 3.77 (3H, s), 3.62 (1H, dd, J=2.83 Hz, 11.25 Hz), 3.30-3.08 (3H, m), 2.93 (1H, d, J=9.96 Hz), 2.50 (1H, d, J=17.86 Hz), 2.27 (3H, s), 2.16 (3H, s), 2.09 (3H, s), 1.39 (1H, d, J=10.35 Hz) ppm; ¹³C NMR(CDCl₃, 100 MHz) δ 150.2, 148.7, 148.3, 145.5, 138.3, 137.1, 135.8, 130.5, 129.6, 128.8, 128.6, 128.4, 127.9, 127.1, 126.7, 125.8, 124.7, 120.1, 113.3, 111.1, 107.4, 100.0, 75.1, 73.7, 70.4, 62.9, 60.7, 60.1, 59.3, 58.2, 55.7, 51.0, 41.9, 25.3, 15.9, 8.80 ppm; IR (NaCl) 3436, 2918, 2357, 1455, 1385, 1325, 1237, 1109, 1072 cm⁻¹; HRMS (FAB) calcd for C₄₀H₄₁N₃O₇ [M+H]: 675.29; found 675.2941; [α]_{D}¹⁷+4.37 (c=1.0 CHCl₃).

### 5.19 Example 19

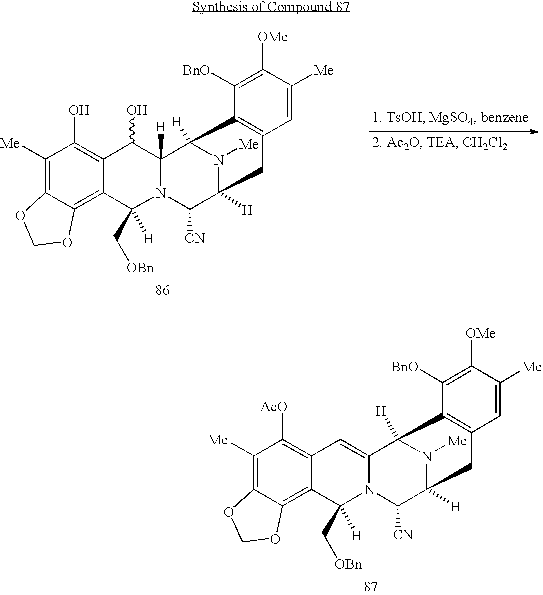

To solution of Compound 86 (14.0 mg, 0.0207 mmoles) and MgSO₄ (10.0 mg, 0.0839 mmoles, 4 equiv) in 1.5 mL of benzene, at 25° C., was added TsOH (59.0 mg, 0.311 mmoles, 15 equiv). The solution was kept for 1 hour at room temperature. The reaction was then diluted with H₂O and extracted with CH₂Cl₂ three times. The organic layers were combined, dried with MgSO₄, and concentrated by rotary evaporation. The crude residue was dissolved in 1.5 mL of CH₂Cl₂. To this solution, at room temperature, was added acetic anhydride (19.6 μL, 0.207 mmoles, 10 equiv) and TEA (28.9 μL, 0.207 mmoles, 10 equiv). The solution was kept 5 hours at room temperature. The reaction was then quenched with an aqueous solution of NaHCO₃ and extracted with CH₂Cl₂ three times. The resultant residue was purified by PTLC with 35% EtOAc/Hexanes to provide 13.0 mg (90%) of Compound 87 as a yellowish foam: ¹H NMR (CDCl₃, 400 MHz) δ 7.50 (2H, d, J=7.24 Hz), 7.43 (2H, t, J=7.28 Hz), 7.37-7.26 (4H, m), 7.14 (2H, d, J=7.04 Hz), 6.46 (1H, s), 5.92 (1H, s), 5.88 (1H, s), 5.65 (1H, s), 5.12 (1H, d, J=11.24 Hz), 5.03 (1H, d, J=11.24 Hz), 4.61 (1H, dd, J=2.52 Hz, 9.76 Hz), 4.54 (1H, s), 4.19 (1H, s), 4.02 (1H, d, J=11.96 Hz), 3.99 (1H, d, J=11.96 Hz), 3.80 (3H, s), 3.41 (1H, d, J=7.08 Hz), 3.18 (1H, dd, J=2.68 Hz, 9.36 Hz), 3.11-2.98 (2H, m), 2.62 (1H, d, J=17.84 Hz), 2.35 (3H, s), 2.21 (3H, s), 1.97 (3H, s), 1.92 (3H, s) ppm; ¹³C NMR (CDCl₃, 100 MHz) δ 168.7, 148.9, 148.6, 140.1, 138.1, 137.8, 130.4, 128.4, 128.0, 127.6, 127.3, 126.7, 126.4, 124.8, 118.9, 118.0, 112.3, 105.6, 101.2, 96.7, 74.1, 73.2, 72.8, 60.4, 58.3, 56.8, 56.6, 56.3, 41.7, 26.6, 20.1, 16.0, 9.8 ppm; IR (NaCl) 2929, 1762, 1455, 1368, 1317, 1202, 1120, 1074, 1028 cm⁻¹; HRMS (FAB) calcd for C₄₂H₄₁N₃O₇ [M+H]: 699.29; found 700.3031; [α]_{D}⁷+80.21 (c=1.0 CHCl₃).

### 5.20 Example 20

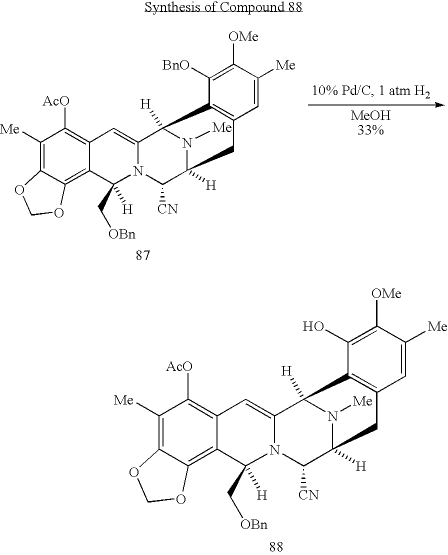

To solution of Compound 87 (28.0 mg, 0.0400 mmoles) in 3 mL of MeOH, at 25° C., was added 30% Pd/C (5.6 mg, 20% w/w), and the reaction was kept under 1 atm of H₂ for 6 hours at room temperature. The reaction mixture was then filtered through cotton and concentrated. The resultant residue was purified by PTLC with 50% EtOAc/Hexanes to provide 8 mg (32.8%) of Compound 88 as a yellow film: ¹H NMR (CDCl₃, 400 MHz) δ 7.34 (3H, m), 7.15 (2H, d, J=7.60 Hz), 6.23 (1H, s), 5.93 (1H, s), 5.87 (1H, s), 5.72 (1H, s), 5.68, (1H, s), 4.63 (1H, dd, J=2.40 Hz, 9.60 Hz), 4.50 (1H, s), 4.25 (1H, s), 4.11 (1H, d, J=12.00 Hz), 3.98 (1H, d, J=12.00 Hz), 3.78 (3H, s), 3.43 (1H, d, J=7.60 Hz), 3.18 (1H, dd, J=2.80 Hz, 9.20 Hz), 3.11-2.98 (2H, m), 2.59 (1H, d, J=17.60 Hz), 2.48 (3H, s), 2.38 (3H, s), 2.21 (3H, s), 1.97 (3H, s) ppm; ¹³C NMR (CDCl₃, 100 MHz) δ 169.3, 145.8, 144.5, 143.1, 140.4, 138.3, 137.8, 136.1, 128.4, 128.1, 127.4, 127.0, 121.0, 119.5, 119.3, 118.4, 112.3, 105.8, 101.3, 95.8, 73.1, 73.0, 60.8, 57.5, 56.6, 55.8, 41.5, 29.7, 26.9, 20.4, 15.8, 9.5 ppm; IR (NaCl) 3441, 2918, 2363, 1769, 1638, 1462, 1434, 1374, 1198, 1121, 1083 cm⁻¹; HRMS (FAB) calcd for C₃₅H₃₅N₃O₇ [M+Hf]: 609.25; found 610.2531; [α]_{D}¹⁷+69.28 (c=1.0 CHCl₃).

### 5.21 Example 21

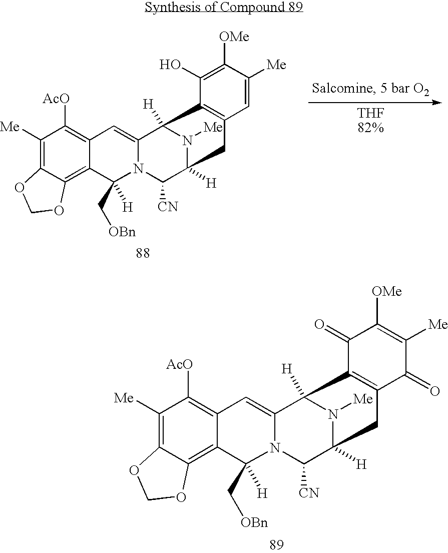

To solution of Compound 88 (11.2 mg, 0.0184 mmoles) in 3 mL of THF, at 25° C., was added, salcomine (1 mg, 0.00308 mmoles, 0.17 equiv) and the reaction was kept under 5 bar of O₂ for 12 hours at room temperature. The reaction mixture was then filtered through cotton and concentrated. The resultant residue was purified by PTLC with 50% EtOAc/Hexanes to provide 9.4 mg (82.2%) of Compound 89 as a purple film: ¹H NMR (CDCl₃, 400 MHz) δ 7.34 (3H, m), 7.18 (2H, d, J=6.96 Hz), 5.93 (1H, s), 5.87 (1H, s), 5.54, (1H, s), 4.63 (1H, dd, J=2.61 Hz, 9.46 Hz), 4.42 (1H, s), 4.29 (1H, d, J=11.64 Hz), 4.19 (1H, d, J=11.64 Hz), 4.16 (1H, s), 4.00 (3H, s), 3.42 (1H, d, J=7.33 Hz), 3.30 (2H, m), 2.79 (1H, dd, J=7.53 Hz, 20.73 Hz), 2.48 (1H, d, J=20.72 Hz), 2.42 (3H, s), 2.37 (3H, s), 1.97 (3H, s), 1.96 (3H, s) ppm; ¹³C NMR (CDCl₃, 100 MHz) δ 186.9, 181.5, 179.1, 155.5, 137.4, 137.1, 136.4, 132.6, 128.5, 127.9, 127.8, 118.6, 117.7, 112.7, 105.6, 101.5, 98.9, 73.6, 72.9, 61.0, 57.3, 56.4, 55.6, 53.8, 41.3, 21.8, 20.4, 9.5, 8.7 ppm; IR (NaCl) 2926, 2852, 1762, 1654, 1457, 1198, 1075 cm⁻¹; HRMS (FAB) calcd for C₃₅H₃₃N₃O₈ [M+H]: 623.23; found 624.2360; [α]_{D}¹⁷−70.20 (c=1.0 CHCl₃).

### 5.21 Example 21

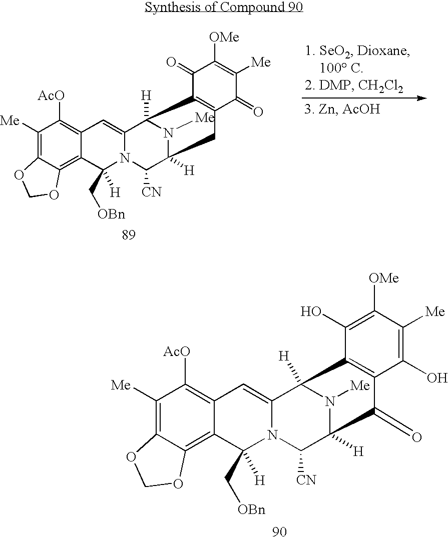

To solution of Compound 89 (13.0 mg, 0.0 mmoles) in 3 mL of dioxane was added selenium dioxide (1 mg, 0.00308 mmoles, 0.17 equiv). The solution was kept for 12 hours at 100° C., in an oil bath. The reaction mixture was quenched with NaHCO₃ and extracted with CH₂Cl₂ three times. The organic layers were combined, dried with MgSO₄, and concentrated in vacuo. To the resultant residue was added 2 mL of CH₂Cl₂ and DMP at room temperature, and the mixture was stirred at room temperature for 12 hours. The reaction was quenched with 10% NaS₂O₃ in saturated NaHCO₃ and extracted with CH₂Cl₂ three times. The resultant residue was then dissolved in 1 mL of AcOH and zinc was added at room temperature. The reaction mixture was stirred vigorously for 15 minutes. The reaction mixture was filtered through cotton, quenched with NaHCO₃, and extracted with CH₂Cl₂ three times. The resultant residue was purified by PTLC with 50% EtOAc/Hexanes to provide 9.4 mg (82.2%) of Compound 90 as a purple film: ¹H NMR (CDCl₃, 400 MHz) δ 7.34 (3H, m), 7.18 (2H, d, J=6.96 Hz), 5.93 (1H, s), 5.87 (1H, s), 5.54, (1H, s), 4.63 (1H, dd, J=2.61 Hz, 9.46 Hz), 4.42 (1H, s), 4.29 (1H, d, J=11.64 Hz), 4.19 (1H, d, J=11.64 Hz), 4.16 (1H, s), 4.00 (3H, s), 3.42 (1H, d, J=7.33 Hz), 3.30 (2H, m), 2.79 (1H, dd, J=7.53 Hz, 20.73 Hz), 2.48 (1H, d, J=20.72 Hz), 2.42 (3H, s), 2.37 (3H, s), 1.97 (3H, s), 1.96 (3H, s) ppm; ¹³C NMR (CDCl₃, 100 MHz) δ 186.9, 181.5, 179.1, 155.5, 137.4, 137.1, 136.4, 132.6, 128.5, 127.9, 127.8, 118.6, 117.7, 112.7, 105.6, 101.5, 98.9, 73.6, 72.9, 61.0, 57.3, 56.4, 55.6, 53.8, 41.3, 21.8, 20.4, 9.5, 8.7 ppm; IR (NaCl) 2926, 2852, 1762, 1654, 1457, 1198, 1075 cm⁻¹; HRMS (FAB) calcd for C₃₅H₃₃N₃O₈ [M+H]: 623.23; found 624.2360; [α]_{D}¹⁷−70.20 (c=1.0 CHCl₃).

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which have been incorporated by reference herein in their entirety.

What is claimed:

1. A method for making a compound having the formula: 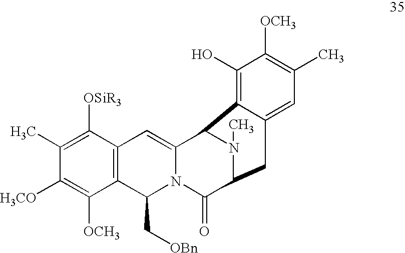
wherein each R is independently —C₁-C₁₂ alkyl or phenyl,
the method comprising allowing a compound of formula 34: 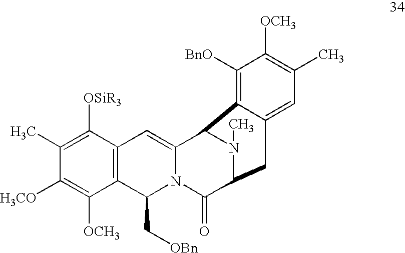
wherein each R is independently —C₁-C₁₂ alkyl or phenyl,
to react with H₂ in the presence of Pd/C under conditions that are sufficient to make the compound of formula 35.

2. The method of claim 1, wherein the Pd/C has about 5% palladium by weight of the Pd/C, and wherein the method further comprising allowing a compound of formula 34 to react in the presence of ethyl acetate.

3. A method for making a compound having the formula: 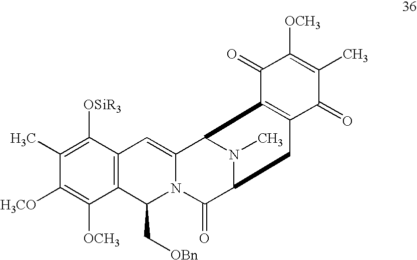
wherein each R is independently —C₁-C₁₂ alkyl or phenyl,
the method comprising allowing a compound of formula 35: 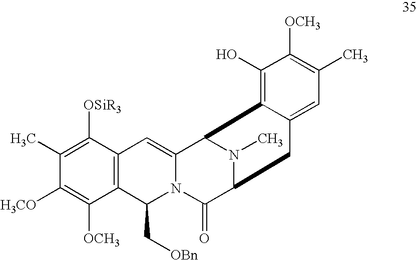
wherein each R is independently —C₁-C₁₂ alkyl or phenyl,
to react with (a) (KSO₃)₂NO, (b) DDQ, or (c) O₂ in the presence of a metal, under conditions that are sufficient to make the compound of formula 36.

4. The method of claim 3, wherein the compound of formula 35 is allowed to react with (KSO₃)₂NO, and wherein the method further comprises allowing the compound of formula 35 to react in the presence of acetonitrile and water.

5. The method of claim 3, wherein the compound of formula 35 is allowed to react with DDQ.

6. A method for making a compound having the formula: 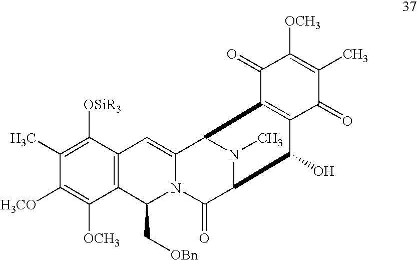
wherein each R is independently —C₁-C₁₂ alkyl or phenyl,
the method comprising allowing a compound of formula 36: 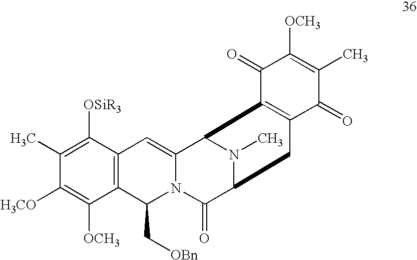
wherein each R is independently —C₁-C₁₂ alkyl or phenyl,
to react with SeO₂ under conditions that are sufficient to make the compound of formula 37.

7. The method of claim 6, wherein the method further comprises allowing a compound of formula 36 to react in the presence of dioxane.

8. A method for making a compound having the formula: 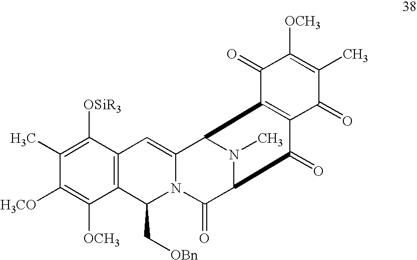
wherein each R is independently —C₁-C₂ alkyl or phenyl,
the method comprising allowing a compound of formula 37: 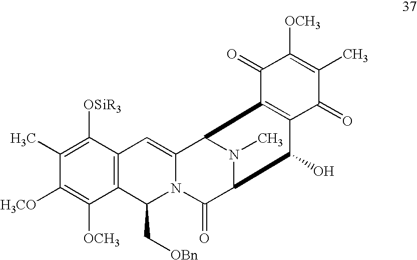
wherein each R is independently —C₁-C₁₂ alkyl or phenyl,
to react with an oxidizing agent under conditions that are sufficient to make the compound of formula 38.

9. The method of claim 8, wherein oxidizing agent is the Dess-Martin periodinane and wherein the method further comprises allowing a compound of formula 37 to react in the presence of methylene chloride.

10. A method for making a compound having the formula: 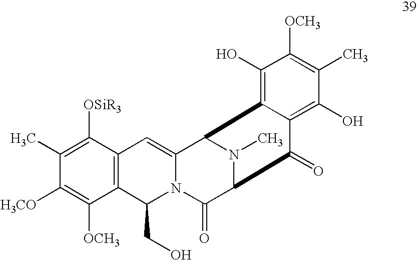
wherein each R is independently —C₁-C₁₂ alkyl or phenyl,
the method comprising allowing a compound of formula 38: 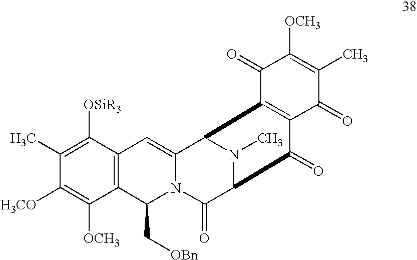
wherein each R is independently —C₁-C₁₂ alkyl or phenyl,
to react with 12 in the presence of Pd/C under conditions that are sufficient to make the compound of formula 39.

11. The method of claim 10, wherein the Pd/C has about 10% palladium by weight of the Pd/C, and wherein the method further comprises allowing a compound of formula 38 to react in the presence of methanol or ethanol.

12. A method for making a compound having the formula: 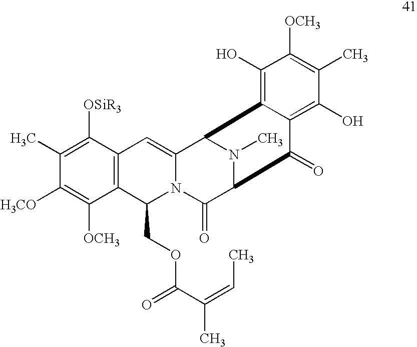
wherein each R is independently —C₁-C₁₂ alkyl or phenyl,
the method comprising allowing a compound of formula 39: 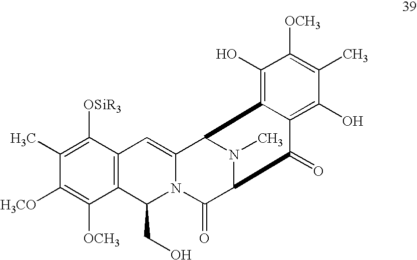
wherein each R is independently —C₁-C₁₂ alkyl or phenyl,
to react with a compound of formula 40: 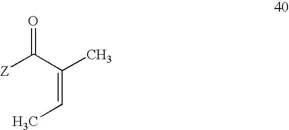
wherein Z is —Cl, —Br, —OH, —C(O)(C₁-C₁₂ alkyl), —C(O)-phenyl, or 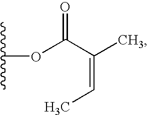
wherein phenyl is unsubstituted or substituted with up to 3 substituents independently selected from -halo, —C₁-C₁₂ alkyl, —O—(C₁-C₁₂ alkyl), —CN, —CF₃, or —NO₂, under conditions that are sufficient to make the compound of formula 41.

13. The method of claim 12, wherein Z is —Cl.

14. The method of claim 13, wherein the method further comprises allowing a compound of formula 39 to react in the presence of methylene chloride.

15. A method for making the compound: 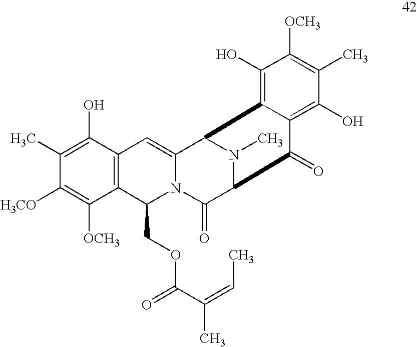
the method comprising allowing a compound of formula 41: 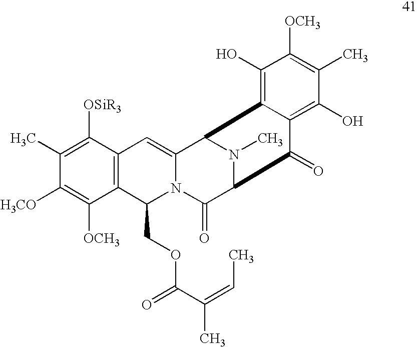
wherein each R is independently —C₁-C₁₂ alkyl or phenyl,
to react with a Bronsted acid or a fluoride salt under conditions that are sufficient to make compound 42.

16. The method of claim 15, wherein the fluoride salt is tetra-n-butylammonium fluoride.

17. The method of claim 15, wherein the method is performed in the presence of acetic acid.

18. The method of claim 15, wherein the method further comprises allowing a compound of formula 41 to react in the presence of tetrahydrofuran.

19. A method for making a compound having the formula: 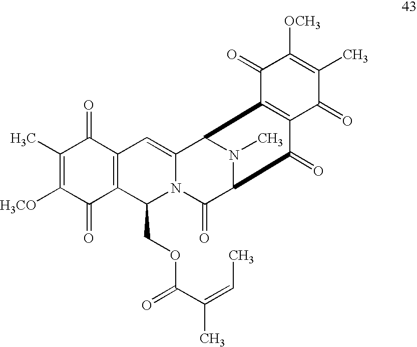
the method comprising allowing Compound 42: 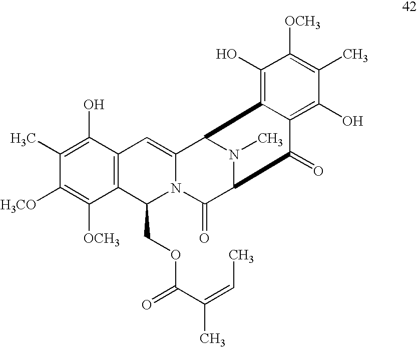
to react with (a) silver oxide, (b) phenyliodonium(III) diacetate, (c) ceric(IV) ammonium nitrate, (d) (KSO₃)₂N0, (e) PhSeCl, (f) MnO₂, (g) phenyliodine (III) bis(trifluoroacetate), or (h) O₂ in the presence of a metal, under conditions that are sufficient to make Compound 43.

20. The method of claim 19, wherein the compound of formula 42 is allowed to react with phenyliodine (III) bis(trifluoroacetate), and wherein the method further comprises allowing a compound of formula 42 to react in the presence of acetonitrile and water.

21. The method of claim 19, wherein the compound of formula 42 is allowed to react with silver oxide.

22. A method for making the compound: 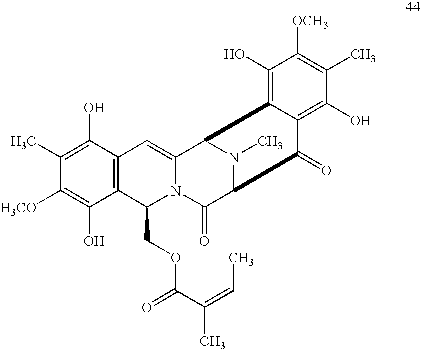
the method comprising allowing Compound 43: 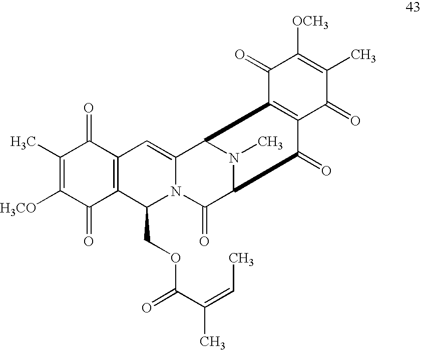
to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44.

23. The method of claim 22, wherein the zinc metal is in the form of zinc dust.

24. The method of claim 22, wherein the Bronsted acid is a carboxylic acid.

25. The method of claim 24, wherein the carboxylic acid is acetic acid.

26. A method for making the compound: 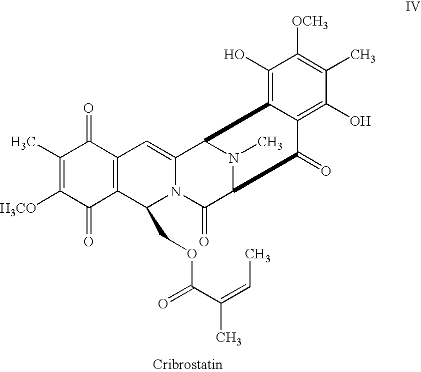
the method comprising allowing Compound 44: 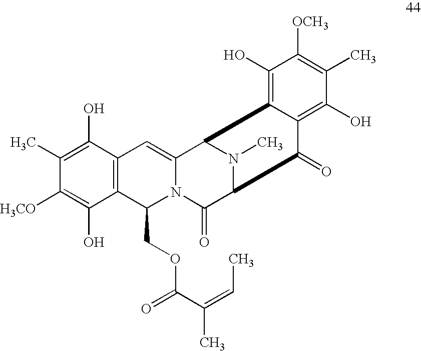
to react with O₂ under conditions that are sufficient to make Cribrostatin IV.

27. The method of claim 26, wherein the method further comprises allowing a compound of formula 44 to react in the presence of N,N-dimethylformamide.

28. The method of claim 26, wherein the compound of formula 44 is allowed to react with O₂ by bubbling air through a mixture of Compound 44 and a reaction solvent.

29. A method for making the compound: 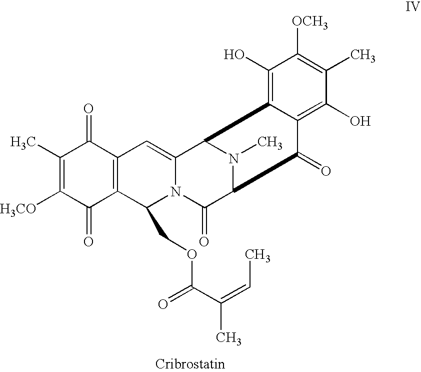
the method comprising the steps:
(i) allowing a compound of formula 34: 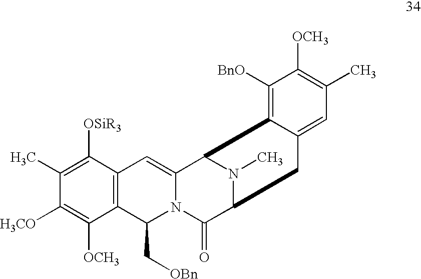
wherein each R is independently —C₁-C₁₂ alkyl or phenyl,
to react with H₂ in the presence of Pd/C under conditions that are sufficient to make a compound of formula 35: 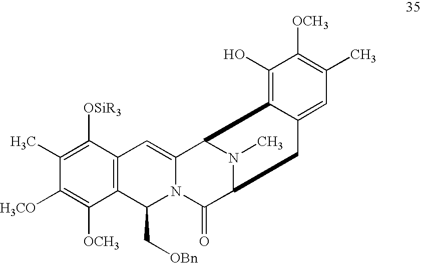
wherein each R is independently —C₁-C₁₂ alkyl or phenyl,
(ii) allowing a compound of formula 35 to react with (a) (KSO₃)₂NO, (b) DDQ, or (c) O₂ in the presence of a metal, under conditions that are sufficient to make a compound of formula 36: 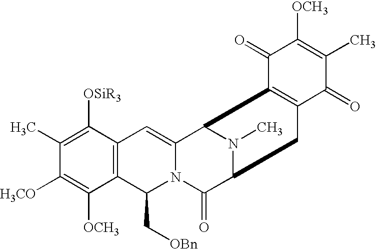
wherein each R is independently —C₁-C₁₂ alkyl or phenyl,
(iii) allowing a compound of formula 36 to react with SeO₂ under conditions that are sufficient to make a compound of formula 37: 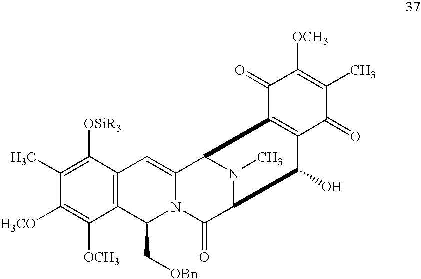
wherein each R is independently —C₁-C₁₂ alkyl or phenyl,
(iv) allowing a compound of formula 37 to react with an oxidizing agent under conditions that are sufficient to make a compound of formula 38: 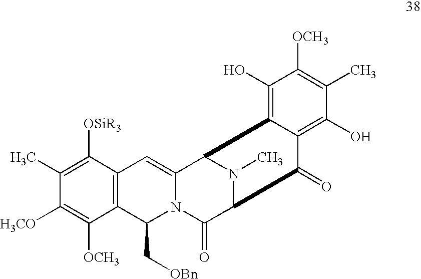
wherein each R is independently —C₁-C₁₂ alkyl or phenyl,
(v) allowing a compound of formula 38 to react with H₂ in the presence of Pd/C under conditions that are sufficient to make a compound of formula 39: 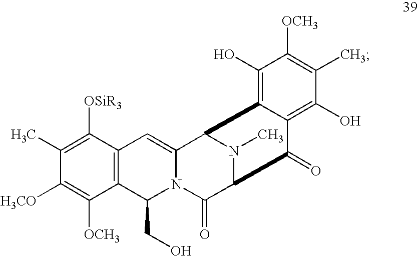
wherein each R is independently —C₁-C₁₂ alkyl or phenyl,
(vi) allowing a compound of formula 39 to react with a compound of formula 40: 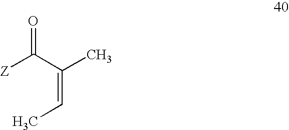
wherein Z is —Cl, —Br, —OH, —C(O)(C₁-C₁₂ alkyl), —C(O)-phenyl, or 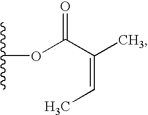
wherein phenyl is unsubstituted or substituted with up to 3 substituents independently selected from -halo, —C₁-C₁₂ alkyl, —O—(C₁-C₁₂ alkyl), —CN, —CF₃, or —NO₂, under conditions that are sufficient to make a compound of formula 41: 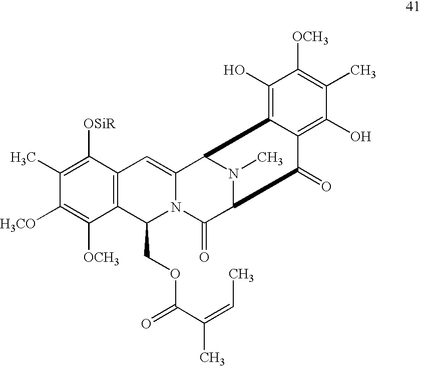
wherein each R is independently —C₁-C₁₂ alkyl or phenyl,
(vii) allowing a compound of formula 41 to react with a Bronsted acid or a fluoride salt under conditions that are sufficient to make Compound 42. 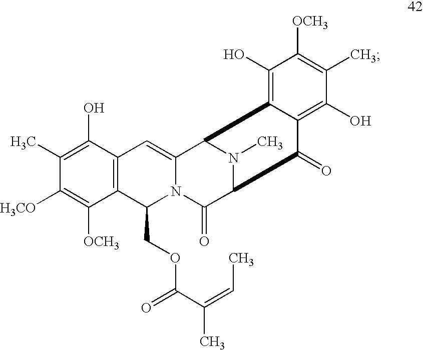
(viii) allowing Compound 42 to react with (a) silver oxide, (b) phenyliodonium(III) diacetate, (c) ceric(IV) ammonium nitrate, (d) (KSO₃)₂NO, (e) PhSeCl, (f) MnO₂, (g) phenyliodine (III) bis(trifluoroacetate), or (h) O₂ in the presence of a metal, under conditions that are sufficient to make Compound 43: 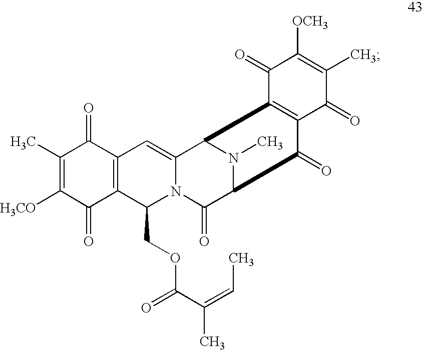
(ix) allowing Compound 43 to react with (a) Na₂S₂O₃, (b) NaBH₄, (c) NaHSO₃, (d) Na₂(SO₂)₂, or (e) a mixture of zinc metal and a Bronsted acid, under conditions that are sufficient to make Compound 44: 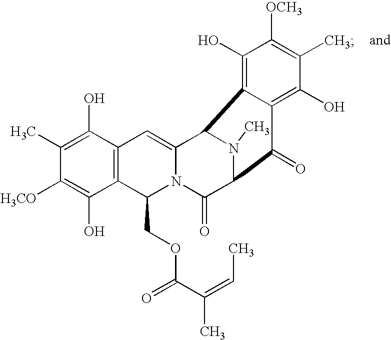
(x) allowing Compound 44 to react with O₂ under conditions that are sufficient to make Cribrostatin IV.

30. A compound having the formula: 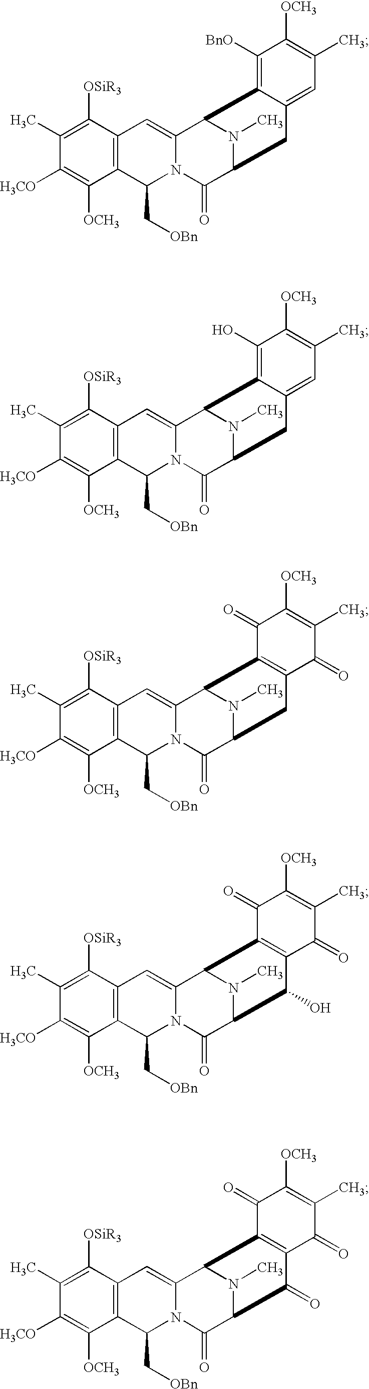 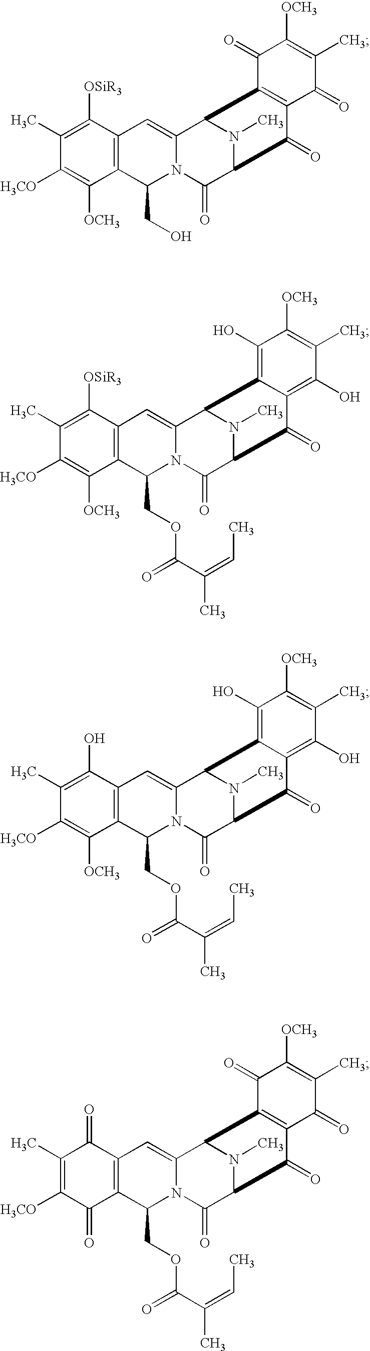 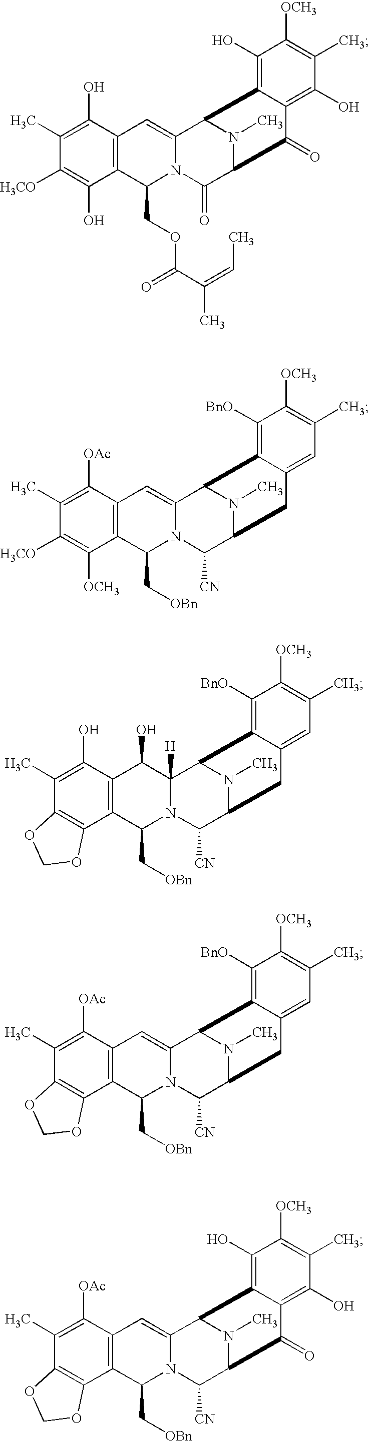 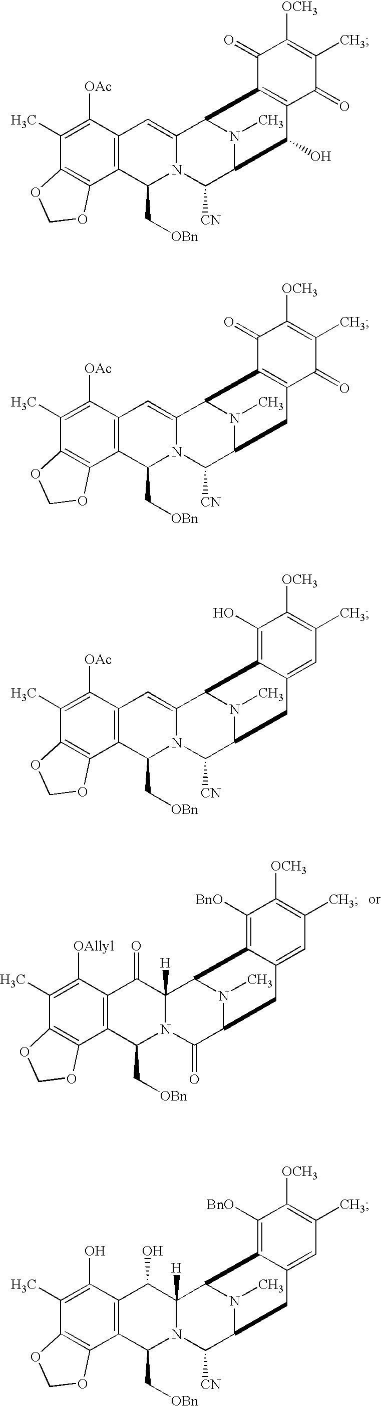
wherein each occurrence of R is independently —C₁-C₁₂ alkyl or -phenyl.

31. A compound having the formula: 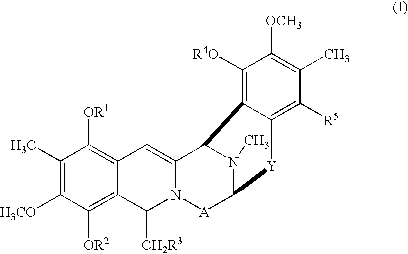
or a pharmaceutically acceptable salt thereof,
wherein:
R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R¹ is independently —H or unsubstituted —C₁-C₆ alkyl;
R² is —H or —C₁-C₁₂ alkyl;
R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl, or 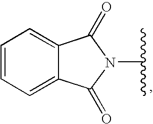
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R⁴ is —H, —C₁-C₁₂ alkyl, or -benzyl;
R⁵ is —H, —OH or —O—C₁-C₁₂ alkyl;
A is —CH₂—, —CH(α-OH)—, —CH(α-CN)— or —C(O)—; and
Y is —CH₂—, —CH(α-OH)— or —C(O)—.

32. A compound having the formula: 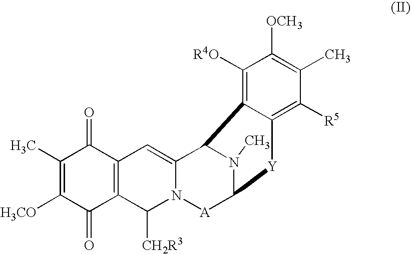
or a pharmaceutically acceptable salt thereof,
wherein:
R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl, —O-benzyl or 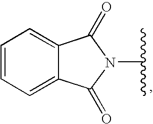
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R⁴ is —H, —C₁-C₁₂ alkyl, or -benzyl;
R⁵ is —H, —OH or —O—C₁-C₁₂ alkyl;
A is —CH₂—, —CH(α-OH)—, —CH(α-CN)— or —C(O)—; and
Y is —CH₂—, —CH(α-OH)— or —C(O)—.

33. A compound having the formula: 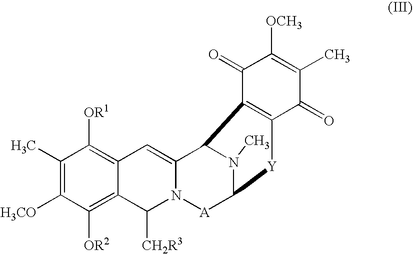
or a pharmaceutically acceptable salt thereof,
wherein:
R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R¹ is —H or —C₁-C₁₂ alkyl;
R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or 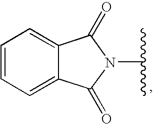
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
A is —CH₂—, —CH(α-OH)—, —CH(α-CN)— or —C(O)—; and
Y is —CH₂—, —CH(α-OH)— or —C(O)—.

34. A compound having the formula: 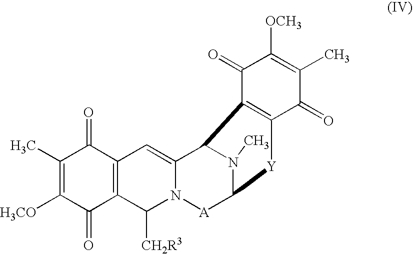
or a pharmaceutically acceptable salt thereof,
wherein:
R³ is —OC(O)—(C₁-C₁₂ alkyl), —NHC(O)—(C₁-C₁₂ alkyl), —NHC(O)-aryl, —C(O)—HN—C(O)—(C₁-C₁₂ alkyl), —O-aryl, —O-benzyl or 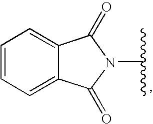
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
A is —CH₂—, —CH(α-OH)—, —CH(α-CN)— or —C(O)—; and
Y is —CH₂—, —CH(α-OH)— or —C(O)—.

35. A compound having the formula: 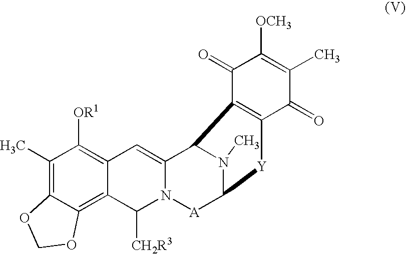
or a pharmaceutically acceptable salt thereof,
wherein:
R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or 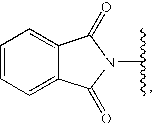
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
A is —CH₂—, —CH(α-OH)—, —CH(α-CN)— or —C(O)—; and
Y is —CH₂—, —CH(α-OH)— or —C(O)—.

36. A compound having the formula: 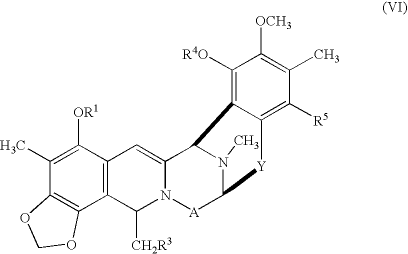
or a pharmaceutically acceptable salt thereof,
wherein:
R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or 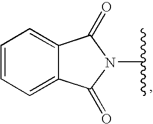
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R⁴ is —H or —C₁-C₁₂ alkyl, or -benzyl;
R⁵ is —H, —OH or —O—C₁-C₁₂ alkyl;
A is —CH₂—, —CH(α-OH)—, —CH(α-CN)— or —C(O)—; and
Y is —CH₂—, —CH(α-OH)— or —C(O)—.

37. A compound having the Formula (VII): 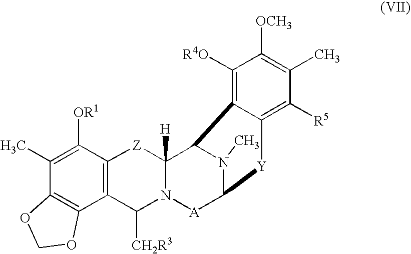
or a pharmaceutically acceptable salt thereof,
wherein:
R¹ is —H, —C₁-C₁₂ alkyl, -allyl, —C(O)—(C₁-C₁₂ alkyl), —C(O)-aryl or —SO₂CH₃, wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R³ is —OC(O)—(C₁-C₁₂ alkyl), —NHC(O)—(C₁-C₁₂ alkyl), —NHC(O)-aryl, —NHC(O)—C(O)—(C₁-C₁₂ alkyl), —C(O)—HN—C(O)—(C₁-C₁₂ alkyl), —O-aryl, —O-benzyl or 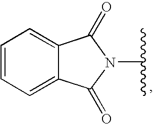
wherein the aryl group is unsubstituted or substituted with one or more of -halo, —O—(C₁-C₆ alkyl), —OH, —CN, —COOR′, —OC(O)R′, —N(R′)₂, —NHC(O)R′ or —C(O)NHR′, wherein each R′ is independently —H or unsubstituted —C₁-C₆ alkyl;
R⁴ is —H or —C₁-C₁₂ alkyl, or -benzyl;
R⁵ is —H, —OH or —O—C₁-C₁₂ alkyl;
A is —CH₂—, —CH(α-OH)—, —CH(α-CN)— or —C(O)—;
Y is —CH₂—, —CH(α-OH)— or —C(O)—; and
Z is —C(O)— or CH(OH)—.

38. A method for treating cancer comprising administering to a subject in need thereof an amount of a compound or pharmaceutically acceptable salt of a compound of claim 31 that is effective to treat cancer.

39. A method for treating cancer comprising administering to a subject in need thereof an amount of a compound or pharmaceutically acceptable salt of a compound of claim 32 that is effective to treat cancer.

40. A method for treating cancer comprising administering to a subject in need thereof an amount of a compound or pharmaceutically acceptable salt of a compound of claim 33 that is effective to treat cancer.

41. A method for treating cancer comprising administering to a subject in need thereof an amount of a compound or pharmaceutically acceptable salt of a compound of claim 34 that is effective to treat cancer.

42. A method for treating cancer comprising administering to a subject in need thereof an amount of a compound or pharmaceutically acceptable salt of a compound of claim 35 that is effective to treat cancer.

43. A method for treating cancer comprising administering to a subject in need thereof an amount of a compound or pharmaceutically acceptable salt of a compound of claim 36 that is effective to treat cancer.

44. A method for treating cancer comprising administering to a subject in need thereof an amount of a compound or pharmaceutically acceptable salt of a compound of claim 37 that is effective to treat cancer.

45. The method of claim 38 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

46. The method of claim 39 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

47. The method of claim 40 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

48. The method of claim 41 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

49. The method of claim 42 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

50. The method of claim 43 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

51. The method of claim 44 wherein the cancer is colorectal cancer, lung cancer, pancreatic cancer, esophageal cancer, stomach cancer, skin cancer, leukemia, lymphoma, testicular cancer, bladder cancer, breast cancer, prostate cancer, head and neck cancer or ovarian cancer.

52. A composition comprising an effective amount of a compound or pharmaceutically acceptable salt of a compound of claim 31 and a physiologically acceptable carrier or vehicle.

53. A composition comprising an effective amount of a compound or pharmaceutically acceptable salt of a compound of claim 32 and a physiologically acceptable carrier or vehicle.

54. A composition comprising an effective amount of a compound or pharmaceutically acceptable salt of a compound of claim 33 and a physiologically acceptable carrier or vehicle.

55. A composition comprising an effective amount of a compound or pharmaceutically acceptable salt of a compound of claim 34 and a physiologically acceptable carrier or vehicle.

56. A composition comprising an effective amount of a compound or pharmaceutically acceptable salt of a compound of claim 35 and a physiologically acceptable carrier or vehicle.

57. A composition comprising an effective amount of a compound or pharmaceutically acceptable salt of a compound of claim 36 and a physiologically acceptable carrier or vehicle.

58. A composition comprising an effective amount of a compound or pharmaceutically acceptable salt of a compound of claim 37 and a physiologically acceptable carrier or vehicle.

59. The method of claim 38 further comprising administering an effective amount of another anticancer agent.

60. The method of claim 39 further comprising administering an effective amount of another anticancer agent.

61. The method of claim 40 further comprising administering an effective amount of another anticancer agent.

62. The method of claim 41 further comprising administering an effective amount of another anticancer agent.

63. The method of claim 42 further comprising administering an effective amount of another anticancer agent.

64. The method of claim 43 further comprising administering an effective amount of another anticancer agent.

65. The method of claim 44 further comprising administering an effective amount of another anticancer agent.
